(19) United States
(12) Patent Application Publication
(10) Pub. No.: US 2026/0325617 A1
(43) Pub. Date: Oct. 22, 2026

(54) INKT Cell Modulator Liposomal Compositions and Methods of Use

(71) Applicant: lOX THERAPEUTICS LIMITED, London (GB)

(72) Inventors: Russell Poe, Newport, RI (US); Ian Walters, Westport, CT (US); Robert Kramer, Heber City, UT (US)

(21) Appl. No.: 19/480,793

(22) PCT Filed: May 3, 2024
(86) PCT No.: PCT/IB2024/054317

§ 371 Date: Oct. 31, 2025

Related U.S. Application Data
(60) Provisional application No. 63/463,868, filed on May 3, 2023.
(60) Provisional application No. 63/463,870, filed on May 3, 2023.
(60) Provisional application No. 63/543,872, filed on Oct. 12, 2023.

(51) Int. Cl. A61K 39/00; A61K 9/00; A61K 9/1271; A61K 9/1277; A61K 9/51; A61K 45/06; A61K 47/02; A61K 47/26; A61P 35/00; B82Y 5/00; C07K 16/28

(52) U.S. Cl. CPC A61K 39/001188; A61K 9/0019; A61K 9/1271; A61K 9/1277; A61K 9/5123; A61K 9/5192; A61K 45/06; A61K 47/02; A61K 47/26; A61P 35/00; C07K 16/2818; C07K 16/2827; A61K 2039/505; A61K 2039/54; A61K 2039/55555; B82Y 5/00

(57) ABSTRACT

Provided herein are compositions including a liposome including Compound A (A), wherein n is 1 (IMM60), 2 (IMM70), or 3 (IMM80), or a salt, ester, solvate, or hydrate thereof, and two or more lipids or salts thereof; and uses thereof, including methods of stimulating an immune response. Also provided herein are liposomes comprising at least one therapeutic agent and a phospholipid bilayer comprising at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG), wherein the phospholipid is substantially devoid of cholesterol, plant sterols, and polyethylene glycolated lipids (PEG lipids).

This application claims priority benefit of U.S. Provisional Patent Application No. 63/543,872, filed on 12 Oct. 2023; No. 63/463,870, filed on 3 May 2023; and No. 63/463,868, filed 3 May 2023. Each of these applications is incorporated by reference in its entirety.

### BACKGROUND

Natural killer T (NKT) cells have been implicated in a range of important immune surveillance mechanisms, such as host defense against external pathogens, immune tolerance and malignancy. NKT cells can be further divided into two subsets, so-named Type I and Type II. Type I NKT cells have received the most attention. These cells are also known as invariant NKT (iNKT) cells owing to their expression of an invariant a chain T cell receptor (TCR; Va14-Ja18 chain in mice and Va24-Ja18 chain in humans), which is paired with a more variable R chain. In contrast, Type II NKT cells have a diverse TCR repertoire and are less well defined, although a subset has been shown to be reactive to sulfatide. The iNKT cell TCR recognizes lipid antigens presented in the context of the non-polymorphic MHC class I-like protein, CD1d. The CD1d molecule has been shown to bind a range of dialkyl lipids and glycolipids and the ensuing iNKT cell TCR recognition of the CD1d-lipid complex leads to the rapid proliferation and release of a plethora of cytokines (both pro-inflammatory and regulatory). The activation of iNKT cells is an important step in ‘boosting’ adaptive immune responses through the activation and maturation of dendritic cells (DC) and B cells through CD40-CD40L interactions, and the activation of natural killer (NK) cells following interferon gamma (IFNγ) release. Since the structure of CD1d ligands has been shown to govern the released cytokine profile, the development of lipid molecules that promote the specific activation of iNKT cells, could find very useful application in the treatment of a wide range of disorders.

Of the range of lipids that bind to CD1d, the glycolipid α-galactosylceramide (α-GalCer) is one of the most potent. α-GalCer is a derivative of the agelasphins, which are naturally occurring glycolipids that were isolated from the marine sponge Agelas mauritianus. Recognition of the α-GalCer-CD1d complex by the iNKT cell TCR results in the secretion of a range of cytokines, and the initiation of a powerful immune response.

While α-GalCer remains one of the most potent iNKT cell agonists and has shown potential in the treatment of various conditions, it may prove difficult to use this molecule widely as a useful therapeutic agent, at least as a direct activator of iNKT cells: not only does α-GalCer-mediated iNKT cell activation lead to the secretion of both T helper Type 1 (Th1) (e.g. IFN-7) and T helper Type 2 (Th2) (e.g. interleukin-4 (IL-4)) cytokines, and therefore a mixed immune response, but more importantly over-stimulation of iNKT cells, which can result in their entering a long-term anergic state, i.e., unresponsiveness to subsequent α-GalCer stimulation and preferential IL-4 production, which would be deleterious for long-term therapy. Loss of circulating levels of iNKT cells could represent a therapeutically significant limitation with iNKT-cell-based therapies if multi-dosing regimens are required. Thus, a need exists for compositions of iNKT cell activators that have effective delivery for clinical applications.

A novel class of iNKT cell agonists is described in International Patent Application No. PCT/EP2012/074140, filed 30 Nov. 2012, published as International Publication No. WO 2013/079687, “INKT Cell Modulators and Methods of Using the Same.” One of these compounds, threitolceramide-(IMM60), has been shown to be very efficient in expanding antigen specific T-cell responses. See Jukes et al., Non-glycosidic compounds can stimulate both human and mouse iNKT cells. Eur J Immunol. 2016; 46(5):1224-1234. Having a threitol moiety at the polar head of IMM60 produces a more stable agonist of iNKTs and therefore increases its bioavailability, resulting in increased maturation and stimulation of the antigen presenting DC.

A need exists for improved formulations of IMM60 and related compounds (e.g., therapeutic agents), and for improved therapeutic combinations and regimens using therapeutic agents (e.g., the disclosed iNKT cell antagonists).

Immune checkpoints have a natural role in a healthy immune system to prevent uncontrolled immune responses that can be detrimental to healthy cells in the body. However, such checkpoints also can inhibit or prevent the immune system from destroying cancer cells in the body.

Immune checkpoint inhibitors represent a relatively new class of drugs that operate by interfering with checkpoint proteins from binding their partner ligand proteins. By interfering with the “off” signal of a checkpoint, these drugs potentiate T cell activity against cancer cells. This class of drugs have been approved for the treatment of a wide variety of cancers. However, checkpoint inhibitors are neither effective against all types of cancer nor effective in all patients with a given type of cancer. A need exists for materials and methods that potentiate the effect of immune checkpoint inhibitors, especially for cancers or patients that are currently considered insensitive to checkpoint inhibitor therapy.

### SUMMARY

The present disclosure has many facets, including liposomes; compositions (such as pharmaceutical compositions) containing liposomes; methods of making the liposomes and compositions; methods of evaluating and optimizing liposome compositions; and methods of using the compositions, including therapeutic and prophylactic methods of preventing, ameliorating, treating, and curing diseases; and methods of modulating an immune response in a mammalian subject. Exemplary embodiments of the invention are summarized or defined by the following numbered paragraphs:
1. A liposome comprised of Compound A:

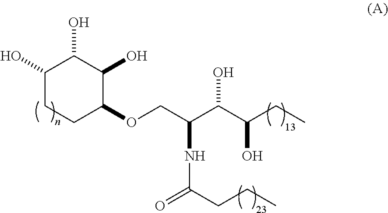

wherein n is 1 (IMM60), 2 (IMM70), or 3 (IMM80), or a salt, ester, solvate, or hydrate thereof, and two or more lipids or salts thereof;
wherein the two or more lipids comprise:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); or,
(f) 1,2-dimyristoyl-sn-glycero-3-phosphoglycerol (DMPG) and cholest-5-en-30-ol (CHOL).
2. The liposome of Embodiment 1, wherein n is 1 (IMM60).
3. The liposome of Embodiment 1 or 2, wherein Compound A is present in an amount of about 0.1 wt. % to about 20 wt. %, based on the total weight of the liposome.
4. The liposome of Embodiment 1 or 2, wherein Compound A is present in an amount of about 0.5 wt. % to about 15 wt. %, or about 1 wt. % to about 12 wt. %, or about 5 wt % to about 12 wt. %, based on the total weight of the liposome.
5. The liposome of any one of Embodiments 1 to 4, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.
6. The liposome of any one of Embodiments 1 to 5, wherein the mass ratio of Compound A to the two or more lipids is about 1:5 to about 1:20, or about 1:7 to about 1:15, or about 1:7 to about 1:12, or about 1:9.

7. The liposome of any one of Embodiments 1 to 6, wherein the two or more lipids comprise 1,2-distearoyl-sn-glycero-3-phosphocholine (DSPC) and 1,2-distearoyl-sn-glycero-3-phospho-rac-glycerol (DSPG).
8. The liposome of Embodiment 7, wherein the DSPC is present in an amount of about 40 wt. % to 80 wt. %, or about 40 wt. % to about 60 wt. %, or about 45 wt. % to about 55 wt. %, based on the total weight of the liposome.
9. The liposome of Embodiment 7 or 8, wherein the DSPG is present in an amount of about 20 wt. % to 60 wt. %, or about 30 wt. % to about 50 wt. %, or about 35 wt. % to about 45 wt. %, based on the total weight of the liposome.
10. The liposome of any one of Embodiments 7-9, wherein the liposome further comprises cholest-5-en-30-ol (CHOL).
11. The liposome of Embodiment 10, wherein the CHOL is present in an amount of about 1 wt % to about 20 wt %, or about 5 wt % to about 15 wt %, or about 8 wt % to about 12 wt %, based on the total weight of the liposome.
12. The liposome of Embodiment 10 or 11, wherein the DSPC is present in an amount of about 40 wt % to about 50 wt %, the DSPG is present in an amount of about 30 wt % to about 40 wt %, and the CHOL is present in an amount of about 5 wt % to about 15 wt %.
13. The liposome of any one of Embodiments 1 to 6, wherein the two or more lipids comprise 1-palmitoyl-2-oleoyl-sn-glycero-3-phosphocholine (POPC) and 3β-[N-(N′,N′-dimethylaminoethane)-carbamoyl]cholesterol hydrochloride (DC-Chol).
14. The liposome of Embodiment 13, wherein the POPC is present in an amount of about 40 wt. % to 80 wt. %, or about 45 wt. % to about 65 wt. %, or about 50 wt. % to about 60 wt. %, based on the total weight of the liposome.
15. The liposome of Embodiment 13 or 14, wherein the DC-Chol is present in an amount of about 15 wt. % to 55 wt. %, or about 20 wt. % to about 50 wt. %, or about 25 wt. % to about 35 wt. %, based on the total weight of the liposome.
16. The liposome of any one of Embodiments 1 to 6, wherein the two or more lipids comprise 1-palmitoyl-2-oleoyl-sn-glycero-3-phosphocholine (POPC) and dimethyldioctadecylammonium bromide (DDAB).
17. The liposome of Embodiment 16, wherein the POPC is present in an amount of about 40 wt. % to 80 wt. %, or about 45 wt. % to about 65 wt. %, or about 50 wt. % to about 60 wt. %, based on the total weight of the liposome.
18. The liposome of Embodiment lor 17, wherein the DC-Chol is present in an amount of about 15 wt. % to 55 wt. %, or about 20 wt. % to about 50 wt. %, or about 30 wt. % to about 40 wt. %, based on the total weight of the liposome.
19. The liposome of any one of Embodiments 1 to 6, wherein the two or more lipids comprise L-α-phosphatidylglycerol (EPG) and 1,2-dioleoyl-sn-glycero-3-ethylphosphocholine (EPC).
20. The liposome of Embodiment 19, wherein the EPG is present in an amount of about 40 wt. % to 80 wt. %, or about 55 wt. % to about 75 wt. %, or about 60 wt. % to about 70 wt. %, based on the total weight of the liposome.
21. The liposome of Embodiment 19 or 20, wherein the EPC is present in an amount of about 10 wt. % to 50 wt. %, or about 15 wt. % to about 40 wt. %, or about 15 wt. % to about 30 wt. %, based on the total weight of the liposome.
22. The liposome of any one of Embodiments 1 to 6, wherein the two or more lipids comprise 1-palmitoyl-2-oleoyl-sn-glycero-3-phosphocholine (POPC) and 1,2-dioleoyl-3-trimethylammonium-propane (DOTAP).
23. The liposome of Embodiment 22, wherein the POPC is present in an amount of about 40 wt. % to 80 wt. %, or about 40 wt. % to about 65 wt. %, or about 45 wt. % to about 55 wt. %, based on the total weight of the liposome.
24. The liposome of Embodiment 22 or 23, wherein the DOTAP is present in an amount of about 20 wt. % to 50 wt. %, or about 25 wt. % to about 45 wt. %, or about 35 wt. % to about 45 wt. %, based on the total weight of the liposome.
25. The liposome of any one of Embodiments 1 to 6, wherein the two or more lipids comprise 1,2-dimyristoyl-sn-glycero-3-phosphoglycerol (DMPG) and cholest-5-en-30-ol (CHOL).
26. The liposome of Embodiment 25, wherein the DMPG is present in an amount of about 40 wt. % to 80 wt. %, or about 50 wt. % to about 75 wt. %, or about 60 wt. % to about 70 wt. %, based on the total weight of the liposome.
27. The liposome of Embodiment 25 or 26, wherein the CHOL is present in an amount of about 10 wt. % to 50 wt. %, or about 15 wt. % to about 35 wt. %, or about 15 wt. % to about 30 wt. %, based on the total weight of the liposome.
28. The liposome of any one of Embodiments 1 to 27, wherein the liposome further comprises a liposomal buffer.
29. The liposome of Embodiment 28, wherein the liposomal buffer has a pH of about to 7, or about 6.3 to about 6.7.

30. The liposome of Embodiment 29, wherein the liposomal buffer comprises NaCl and sodium phosphate.
31. The liposome of any one of Embodiments 28-30, wherein the liposomal buffer further comprises sucrose.
32. The liposome of any one of Embodiments 1 to 31, wherein the liposome further comprises at least one antigen.
33. The liposome of Embodiment 32, wherein the at least one antigen comprises a member selected from the group consisting of a viral antigen, a bacterial antigen, a fungal antigen, a tumor antigen, and mixtures thereof.
34. The liposome of Embodiment 32 or 33, wherein the at least one antigen comprises at least one tumor antigen.
35. The liposome of Embodiment 34, wherein the tumor antigen is selected from the group consisting of:
(a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).
36. The liposome of any one of Embodiments 3β-35, wherein the antigen is present in the liposome in an amount of about 1 g of antigen to 1-20 g of Compound A.
37. The liposome of any one of Embodiments 1 to 36, further comprising at least one therapeutic agent.
38. A composition comprising liposomes according to any one of Embodiments 1-37 and a pharmaceutically acceptable diluent, excipient, adjuvant, or carrier.
39. The composition of Embodiment 38, wherein the mean diameter of the liposomes is less than 200 nm.
40. The composition of Embodiment 38, wherein the mean diameter of the liposomes is within a range of about 50 nm to 200 nm, or about 50 nm to about 170 nm, or about 75 nm to about 145 nm, or about 90 nm to about 130 nm.
41. The composition according to Embodiment 38, wherein the mean diameter of the liposomes is 110 nm±60 nm or 110 nm±40 nm or 110 nm±20 nm.
42. The composition according to any one of Embodiments 38 to 41, wherein the liposomes have a polydispersity index (PdI) of less than or equal to about 0.40, or less than or equal to about 0.35, or less than or equal to about 0.30, or less than or equal to about 0.25, or less than or equal to about 0.20, or less than or equal to about 0.15.

43. The composition of any one of Embodiments 38 to 42, wherein the composition further comprises at least one antigen.
44. The composition of Embodiment 43, wherein the at least one antigen comprises a member selected from the group consisting of a viral antigen, a bacterial antigen, a fungal antigen, a tumor antigen, and mixtures thereof.
45. The composition of Embodiment 43 or 44, wherein the at least one antigen comprises at least one tumor antigen.
46. The composition of Embodiment 45, wherein the tumor antigen is selected from the group consisting of:
   (a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).
47. The composition of Embodiment 45, wherein the tumor antigen comprises NY-ESO-1 or an antigenic fragment thereof.
48. The composition of any one of Embodiments 38 to 47, further comprising at least one therapeutic agent.
49. The composition of Embodiment 48, wherein the at least one therapeutic agent is selected from the group of an immune modulator, a Toll-like receptor agonist, a Nod ligand, an anti-viral agent, an antifungal agent, an antibiotic, an antiviral antibody, a cancer immune therapeutic, a chemotherapy agent, a kinase inhibitor, a cytotoxic agent, an anti-asthmatic agent, an antihistamine agent, an anti-inflammatory agent, a vaccine adjuvant, a second liposome, an artificial antigen presenting cell, a cytokine or chemokine blocking antibody, and combinations thereof.
50. A method of stimulating an immune response in a mammalian subject comprising administering to the subject the composition of any one of Embodiments 38 to 49.
51. The method according to Embodiment 50, further comprising a step, prior to the administering step, of heating the composition for a time and at a temperature sufficient to reduce average particle size or PdI of the liposomal composition.
52. The method according to Embodiment 50, further comprising a step, prior to the administering step, of heating the composition for 5-15 minutes at a temperature of about 55 to about 65° C.
53. The composition according to any one of Embodiments 38 to 49 for use in stimulating an immune response in a mammalian subject.
54. Use of the composition according to any one of Embodiments 38 to 49 for stimulating an immune response in a mammalian subject.
55. The liposome according to any one of Embodiments 1 to 37 or the composition according to any one of Embodiments 38 to 49 for use in the manufacture of a medicament for stimulating an immune response in a mammalian subject.
56. The composition or use according to any one of Embodiments 53 to 55, wherein the composition is subjected to a heating step prior to its use in stimulating the immune response.
57. The method or use of any one of Embodiments 50-56, wherein the mammalian subject is human.
58. The method or use of any one of Embodiments 50 to 57, wherein the mammalian subject has a cancer.
59. A method of treating a mammalian subject with cancer, comprising administering to the subject a therapeutically effective amount of the composition of any one of Embodiments 38 to 49.

60. The method or use according to Embodiment 58 or 59, wherein the composition is administered by subcutaneous, intravenous, or intratumoral injection.
61. The method according to Embodiment 50 or 60, further comprising a step, prior to the administering step, of heating the composition for a time and at a temperature sufficient to reduce average particle size or PdI of the liposomal composition.
62. The liposome according to any one of Embodiments 1 to 37 or the composition according to any one of Embodiments 38 to 49 for use in the treatment of cancer in a mammalian subject.
63. The liposome according to any one of Embodiments 1 to 37 or the composition according to any one of Embodiments 38 to 49 for use in the manufacture of a medicament for treatment of cancer in a mammalian subject.
64. The composition or use according to any one of Embodiments 61 to 62, wherein the composition is subjected to a heating step prior to its use in stimulating the immune response.
65. The method or use of any one of Embodiments 58 to 64, wherein the cancer is selected from the group of basal cell carcinoma, breast cancer leukemia, Burkitt's lymphoma, colon cancer, esophageal cancer, bladder cancer, gastric cancer, head and neck cancer, hepatocellular cancer, Hodgkin's lymphoma, hairy cell leukemia, Wilms' tumor, thyroid cancer, thymoma and thymic carcinoma, testicular cancer, T-cell lymphoma, prostate cancer, non-small cell lung cancer, liver cancer, renal cell cancer, melanoma, and combinations thereof.
66. The method or use of any one of Embodiments 58 to 64, wherein the cancer comprises melanoma.
67. The method or use of any one of Embodiments 58 to 64, wherein the cancer comprises non-small cell lung cancer.
68. The method or use of any one of Embodiments 58 to 67, further comprising administering to the subject or using at least one further therapeutic or therapy selected from the group consisting of a chemotherapeutic agent, a radiotherapeutic agent, and radiation therapy.
69. The method or use of any one of Embodiments 58-68, further comprising administering to the subject or using at least one checkpoint inhibitor.
70. The method or use of Embodiment 69, wherein the immune checkpoint inhibitor comprises a PD-1 inhibitor, a PD-L1 inhibitor, or a CTLA-4 inhibitor.
71. The method or use of Embodiment 70, wherein the immune checkpoint inhibitor comprises at least one PD-1 inhibitor selected from Pembrolizumab, Nivolumab, Cemiplimab, and Spartalizumab.
72. The method or use of Embodiment 70, wherein the immune checkpoint inhibitor comprises at least one PD-L1 inhibitor selected from Atezolizumab, Avelumab, and Durvalumab.
73. The method or use of Embodiment 70, wherein the immune checkpoint inhibitor comprises at least one CTLA-4 inhibitor selected from Ipilimumab and Tremelimumab.
74. The method or use of any one of Embodiments 68-73, wherein the composition and the further therapeutic or therapy are administered concurrently.
75. The method or use of any one of Embodiments 68-73, wherein the composition and the further therapeutic or therapy are administered separately.
76. A method of preparing a liposome comprised of Compound A:

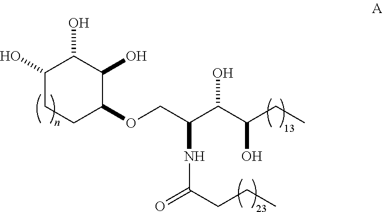

wherein n is 1 (IMM60), 2 (IMM70), or 3 (IMM80), or a salt, ester, solvate, or hydrate thereof, and two or more lipids or salts thereof; the method comprising:
(A) admixing Compound A and the two or more lipids to form multi-lamellar vesicles, wherein the two or more lipids comprise:
   (a) 1,2-distearoyl-sn-glycero-3-phosphocholine (DSPC) and 1,2-distearoyl-sn-glycero-3-phospho-rac-glycerol (DSPG);
   (b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
   (c) 1-palmitoyl-2-oleoyl-sn-glycero-3-phosphocholine (POPC) and dimethyldioctadecylammonium bromide (DDAB);
   (d) L-α-phosphatidylglycerol (EPG) and 1,2-dioleoyl-sn-glycero-3-ethylphosphocholine (EPC);
   (e) 1-palmitoyl-2-oleoyl-sn-glycero-3-phosphocholine (POPC) and 1,2-dioleoyl-3-trimethylammonium-propane (DOTAP); or
   (f) 1,2-dimyristoyl-sn-glycero-3-phosphoglycerol (DMPG) and cholest-5-en-30-ol (CHOL), and,
(B) extruding the multi-lamellar vesicles through a membrane to form the liposomes.
77. The method according to Embodiment 76, further comprising (c) purifying the liposomes.
78. The method according to Embodiment 77, wherein the purifying comprises filtration.
79. The method according to Embodiment 77, wherein the purifying comprises ultrafiltration, centrifugation, dialysis, diafiltration, or tangential flow filtration.
80. The method according to Embodiment 78 that comprises filtering with a filter that has a pore size of 400 nm, or less than about 350 nm, or less than about 300 nm, or less than about 250 nm, or less than about 220 nm, or less than about 200 nm, or less than about 150 nm.
81. The method according to Embodiment 78 that comprises filtering with a filter that has a pore size of about 50 nm to about 250 nm, or about 50 nm to about 150 nm, or about 60 nm to about 100 nm, or about 80 nm.
82. The method according to Embodiment 78 that comprises filtering with a filter that has a molecular weight cutoff (MWCO) of 1000 kD or 800 kD or 600 kD or 500 kD or 400 kD or 300 kD or 250 kD.
83. The method of any one of Embodiments 7-82, wherein n is 1 (IMM60).
84. The method of any one of Embodiments 7-83, wherein Compound A is present in an amount of about 0.1 wt. % to about 20 wt. %, or about 0.5 wt. % to about 15 wt. %, or about 1 wt. % to about 12 wt. %, or about 5 wt % to about 12 wt. %, based on the total weight of the liposome.
85. The method of any one of Embodiments 7-84, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.
86. The method of any one of Embodiments 7-81, wherein the mass ratio of Compound A to the two or more lipids is in a range of about 1:5 to about 1:20, or about 1:7 to about 1:15, or about 1:7 to about 1:12, or about 1:9.
87 The method of any one of Embodiments 7-86, wherein the two or more lipids comprise 1,2-distearoyl-sn-glycero-3-phosphocholine (DSPC) and 1,2-distearoyl-sn-glycero-3-phospho-rac-glycerol (DSPG).
88. The method of Embodiment 87, wherein the DSPC is present in an amount of about 40 wt. % to 80 wt. %, or about 40 wt. % to about 60 wt. %, or about 45 wt. % to about 55 wt. %, based on the total weight of the liposome.
89. The method of Embodiment 87 or 88, wherein the DSPG is present in an amount of about 20 wt. % to 60 wt. %, or about 30 wt. % to about 50 wt. %, or about 35 wt. % to about 45 wt. %, based on the total weight of the liposome.
90. The method of any one of Embodiments 7-84, wherein the two or more lipids further comprise CHOL.
91. The method of any one of Embodiments 7-90, wherein step (A) further includes admixing at least one antigen with the Compound A and the lipids.
92. The method of Embodiment 91, wherein the at least one antigen comprises a viral antigen, a bacterial antigen, a fungal antigen, and/or a tumor antigen.
93. The method of Embodiment 91 or 92, wherein the at least one antigen comprises at least one tumor antigen.
94. The liposome of Embodiment 93, wherein the tumor antigen is selected from the group consisting of:
(a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).
95. The method of any one of Embodiments 7-94 wherein step (A) further comprises admixing at least one therapeutic agent with the lipids and the Compound A.
96. A liposome prepared by the method of any one of the Embodiments 76-95.
97. A method of treating a liposomal formulation, the method comprising:
(a) providing a liposomal composition according to any one of Embodiments 38-49; and
(b) heating the composition for a time and at temperature sufficient to reduce average particle size or PdI of the liposomal composition.
98. A method of treating a liposomal formulation, the method comprising:
(a) providing a liposomal composition according to any one of Embodiments 38-49; and
(b) heating the composition for 5-15 minutes at a temperature of about 55 to about 65° C.
99. The method according to Embodiment 97 or 98, wherein the liposomal composition provided in (a) has been frozen, stored below 0° C. or below −20° C., or freeze dried and reconstituted.
100. A liposome comprised of a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent, wherein the phospholipid bilayer is substantially devoid of cholesterol, plant sterols, and polyethylene glycolated lipids (PEG lipids).
101. The liposome of embodiment 100, wherein the phospholipid bilayer has a phase transition temperature above about 37° C.
102. The liposome of embodiment 100 or 101, wherein the lipid bilayer comprises DSPC and DSPG.
103. The liposome of embodiment 100 or 101, wherein the phospholipids in the phospholipid bilayer consist essentially of DSPC and DSPG.
104. The liposome of embodiment 100 or 101 that is free of phospholipids having a phase transition temperature below about 37° C.
105. The liposome of any one of embodiments 100-104 that is free of unsaturated lipids or unsaturated phospholipids.
106. The liposome of any one of embodiments 99-105 that is free of lipids or phospholipids having a chain length shorter than 16 carbon atoms.
107. The liposome of any one of embodiments 100-106, wherein the at least two phospholipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the phospholipid bilayer.
108. The liposome of any one of embodiments 100-107, further comprising a therapeutic agent.
109. The liposome of any one of embodiments 100-108, wherein the mass ratio of the at least one therapeutic agent to the at least two phospholipids is about 1:5 to about 1:20, or about 1:7 to about 1:15, or about 1:7 to about 1:12, or about 1:9.
110. The liposome of any one of embodiments 100-109 that has a mean diameter less than about 500 nm, or less than about 400 nm, or less than about 300 nm, or less than about 200 nm, or less than about 150 nm, or less than 125 nm, wherein the mean diameter is measured as a Z-average at 90°.
111. The liposome of any one of embodiments embodiment 100-110, wherein the at least one therapeutic agent comprises a hydrophobic or amphiphilic agent that intercalates or integrates into the phospholipid bilayer.
112. The liposome of any one of embodiments 100-111, wherein the at least one therapeutic agent includes a hydrophilic agent contained in the lumen of the liposome.
113. The liposome of any one of embodiments 100-112, wherein the at least one therapeutic agent is selected from small molecules, nucleic acids, RNA, DNA, aptamers, proteins, peptides, and a mixture thereof.
114. The liposome of any one of embodiments 100-113, wherein the at least one therapeutic agent comprises a ribonucleic acid (RNA). In some embodiments, the RNA is an siRNA or an mRNA.
115. The liposome of any one of embodiments 100-114, wherein the at least one therapeutic agent comprises an anti-cancer therapeutic.
116. The liposome of any one of embodiments 100-115, wherein the at least one therapeutic agent comprises an immunostimulant or encodes an immunostimulant.
117. The liposome of any one of embodiments 100-116, wherein the at least one therapeutic agent is present in an amount of about 0.1 wt. % to about 20 wt. % based on the total weight of the liposome.
118. The liposome of any one of embodiments 100-117, wherein the therapeutic agent is present in an amount of about 0.5 wt. % to about 15 wt. %, or about 1 wt. % to about 12 wt. %, or about 5 wt. % to about 12 wt. %, based on the total weight of the liposome.
119. The liposome of any one of claims 100-119, comprising one or more therapeutic agent.
120. The liposome of any one of claims 100-120, wherein the lumen comprises a therapeutic agent.
121. A composition comprising liposomes according to any one of embodiments 100-120 and a pharmaceutically acceptable carrier.
122. The composition according to embodiment 121, wherein the liposomes have a mean diameter of less than or equal to about 500 nm, or less than about 400 nm, or less than about 300 nm, or less than about 200 nm, or less than about 150 nm, or less than 125 nm, wherein the mean diameter is measured as a Z-average at 90°.
123. The composition according to embodiment 121 or 122, wherein the liposomes have a mean diameter of about 100 nm±60 nm, or about 100 nm±40 nm, or about 100 nm±20 nm.
124. The composition of any one of embodiments 121-123, wherein the liposomes have a terminal half-life of at least about 6 hours after the composition is administered to humans.
125. The composition of any one of embodiments 121-124, wherein the liposomes have a terminal half-life of about 6-12 hours after the composition is administered to humans intravenously.
126. The composition of any one of embodiments 121-125, wherein the liposomes have a terminal half-life of about 8 hours after the composition is administered to humans intravenously.
127. The composition of any one of embodiments 121-126, wherein the liposome has a steady state volume of distribution in humans between about 2 liters to about 12 liters, or about 2 liters to about 10 liters, or about 2 liters to about 8 liters, after the composition is administered to humans intravenously.
128. The composition of any one of claims 121-127, wherein the liposome has a terminal volume of distribution in a mammalian subject of about 3 liters to about 10 liters after the composition is administered intravenously.
129. The composition of any one of claims 121-128, wherein the composition provides a steady plasma concentration of therapeutic agent between about 2 hours to about 10 hours, or between about 3 hours to about 8 hours, or between about 4 hours to about 6 hours, after administration to a mammalian subject.
130. The composition of any one of embodiments 121-129, wherein the composition provides a steady plasma concentration of therapeutic agent between about 4 hours and about 6 hours after administration to a mammalian subject.
131. The composition of any one of embodiments 121-130, wherein the therapeutic agent is not substantially cleared from plasma between about 4 hours and about 6 hours after administration to a mammalian subject.
132. The composition of any one of embodiments 128-131, wherein the mammalian subject is human.
133. A method of stimulating an immune response in a mammalian subject comprising administering to the subject the composition of any one of embodiments 121-132.
134. Use of the composition of any one of embodiments 121-132 for stimulating an immune response in a mammalian subject.
135. The liposome of any one of embodiments 100-120 or the composition of any one of embodiments 121-132 for use in the manufacture of a medicament for stimulating an immune response in a mammalian subject.
136. The method or use of any one of embodiments 133-135, wherein the mammalian subject is human.
137. The method or use of any one of embodiments 133-136, wherein the mammalian subject has a cancer.
138. A method of treating a mammalian subject with cancer, comprising administering to the subject a therapeutically effective amount of the composition of any one of embodiments 121-132.
139. The method or use of embodiment 137 or method of embodiment 138, wherein the composition is administered by subcutaneous, intravenous, or intratumoral injection.
140. The liposome of any one of embodiments 100-120 or the composition of any one of embodiments 121-132 for use in the treatment of cancer in a mammalian subject.
141. The liposome of any one of embodiments 100-120 or the composition of any one of embodiments 121-132 for use in the manufacture of a medicament for treatment of cancer in a mammalian subject.
142. The method or use of any one of embodiments 137-141, wherein the cancer is selected from the group of basal cell carcinoma, breast cancer leukemia, Burkitt's lymphoma, colon cancer, esophageal cancer, bladder cancer, gastric cancer, head and neck cancer, hepatocellular cancer, Hodgkin's lymphoma, hairy cell leukemia, Wilms' tumor, thyroid cancer, thymoma and thymic carcinoma, testicular cancer, T-cell lymphoma, prostate cancer, pancreatic cancer, non-small cell lung cancer, liver cancer, renal cell cancer, melanoma, and combinations thereof.
143. The method or use of any one of embodiments 137-142, wherein the cancer comprises or is melanoma.
144. The method or use of any one of embodiments 137-143, wherein the cancer comprises or is non-small cell lung cancer.
145. The method or use of any one of embodiments 137-144, wherein the cancer comprises or is liver cancer.
146. The method or use of any one of embodiments 137-145, further comprising administering to the subject or using at least one further therapeutic or therapy selected from the group consisting of a chemotherapeutic agent, a radiotherapeutic agent, and radiation therapy.
147. The method or use any one of embodiments 137-146, further comprising administering to the subject or using at least one immune checkpoint inhibitor.
148. The method or use of embodiment 147, wherein the immune checkpoint inhibitor comprises a PD-1 inhibitor, a PD-L1 inhibitor, or a CTLA-4 inhibitor.
149. The method or use of embodiment 148, wherein the immune checkpoint inhibitor comprises at least one PD-1 inhibitor selected from Pembrolizumab, Nivolumab, Cemiplimab, and Spartalizumab.
150. The method or use of embodiment 148, wherein the immune checkpoint inhibitor comprises at least one PD-L1 inhibitor selected from Atezolizumab, Avelumab, and Durvalumab.
151. The method or use of embodiment 148, wherein the immune checkpoint inhibitor comprises at least one CTLA-4 inhibitor selected from Ipilimumab and Tremelimumab.
152. The method or use of any one of embodiments 146-151, wherein the composition and the further therapeutic or therapy are administered concurrently.
153. The method or use of any one of embodiments 146-151, wherein the composition and the further therapeutic or therapy are administered separately.
154. A method of preparing a liposome comprising a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent, wherein the phospholipid bilayer is substantially devoid of cholesterol, plant sterols, and polyethylene glycolated lipids (PEG lipids), wherein the method comprises 1) admixing the therapeutic agent and the at least two phospholipids to form multi-lamellar vesicles and 2) extruding the multi-lammellar vesicles through a membrane to form the liposomes.
155. The method of embodiment 154, further comprising purifying the liposomes.
156. The method of embodiment 154 or 155, wherein purifying the liposomes comprises filtration.
157. The method of embodiment 156, wherein purifying comprises ultrafiltration, centrifugation, dialysis, diafiltration, or tangential flow filtration.
158. The method according to any one of embodiments 154-157 comprising filtering with a filter that has a pore size of about 450 nm or less, or about 400 nm or less, or about 350 nm or less, or about 300 nm or less, or about 250 nm or less, or about 220 nm or less, or about 200 nm or less, or about 150 nm or less.
159. The method according to any one of embodiments 154-158, wherein the membrane has a pore size of about 50 nm to about 250 nm, or about 50 nm to about 150 nm, or about 60 nm to about 100 nm, or about 80 nm.
160. The method according to any one of embodiments 154-159 comprising filtering with a filter that has a molecular weight cutoff (MWCO) of about 500 kD, or about 450 kD, or about 400 kD, or about 350 kD, or about 300 kD, or about 250 kD.
161. The method of any one of embodiments 150-156, wherein the therapeutic agent is present in an amount of about 0.1 wt. % to about 20 wt. %, or about 0.5 wt. % to about 15 wt. %, or about 1 wt. % to about 12 wt. %, or about 5 wt % to about 12 wt. %, based on the total weight of the liposome.
162. The method of any one of embodiments 154-161, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.
163. The method of any one of embodiments 154-162, wherein the mass ratio of the therapeutic agent is in a range of about 1:5 to about 1:20, or about 1:7 to about 1:15, or about 1:7 to about 1:12, or about 1:9.
164. The method of any one of embodiments 154-163, wherein the two or more lipids comprise 1,2-distearoyl-sn-glycero-3-phosphocholine (DSPC) and 1,2-distearoyl-sn-glycero-3-phospho-rac-glycerol (DSPG).
165. The method of embodiment 164, wherein the DSPC is present in an amount of about 40 wt. % to 80 wt. %, or about 40 wt. % to about 60 wt. %, or about 45 wt. % to about 55 wt. %, based on the total weight of the liposome.
166. The method of embodiment 164 or 165, wherein the DSPG is present in an amount of about 20 wt. % to 60 wt. %, or about 30 wt. % to about 50 wt. %, or about 35 wt. % to about 45 wt. %, based on the total weight of the liposome.
167. The method of any one of embodiments 154-166, wherein step 1) further includes admixing at least one antigen with the therapeutic agent and the lipids.
168. The method of embodiment 167, wherein the at least one antigen comprises a viral antigen, a bacterial antigen, a fungal antigen, and/or a tumor antigen.
169. The method of embodiment 167 or 168, wherein the at least one antigen comprises at least one tumor antigen.

Included in the disclosure are embodiments relating to the use of checkpoint inhibitors for therapy, including cancer therapy. Historically, checkpoint inhibitors have not been effective for treating all types of cancer or for treating all patients with a particular type of cancer. The iNKT modulator compositions and methods described herein are believed to have the property of expanding the range and/or efficacy of checkpoint inhibitor therapy, and/or expanding the number of cancer patients who should be deemed sensitive to checkpoint inhibitor therapy for purposes of guiding treatment.

In some embodiments, the invention includes a method of treating a mammalian subject with an iNKT cell modulator as part of a cancer therapy to increase the sensitivity of the subject's cancer to a checkpoint inhibitor.

In some variations, the cancer/subject would be scored as insensitive to treatment with the checkpoint inhibitor. The term “insensitive” is intended to define a clinical judgment of expected ineffectiveness at treating the cancer, based on parameters used to classify treatment of cancer patients as a group, and is not intended as a definitive statement about whether any particular individual cancer patient ultimately would or would not benefit from the checkpoint therapy (if administered). For example, in some variations, a subject/cancer is scored as “insensitive” based on clinical experience a checkpoint inhibitor was ineffective against a cancer in pre-clinical animal experiments and/or human clinical trials and/or published post-approval evidence from the clinic involving similar cancers, and similar subjects, which may optionally take into consideration previous treatments that the subject has received for the cancer. In some variations, a subject is scored as insensitive by virtue of failing to satisfy an established prescribing guideline for a particular checkpoint inhibitor drug. Prescribing guidelines are exemplified herein with reference to some specific guidelines from the U.S. Food and Drug Administration. However, guidelines from regulatory bodies from other jurisdictions are also contemplated and intended as suitable tools for scoring subjects as sensitive or insensitive.

In some variations, a subject is scored as “insensitive” because the subject has previously received the checkpoint inhibitor, and did not respond to the treatment, or stopped responding to the treatment.

In still further variations, PD-L1 expression in the cancer is used as an indicator of sensitivity of the cancer, with a percentage of cancer cells and/or immune cells (especially tumor-associated or tumor-infiltrating immune cells) is used as a cutoff for scoring sensitivity. A cancer with PD-L1 expression on a percentage of cells below the cutoff is scored as insensitive.

The INKT cell modulators described herein are contemplated for sensitizing cancer patients to effective treatment with any checkpoint inhibitor, and several exemplary checkpoint inhibitors are described herein. In some variations, the checkpoint inhibitor targets the Programmed Cell Death Protein 1 (PD-1) pathway.

In some variations, the method involves first identifying a mammalian subject as having a cancer that is insensitive to treatment with an immune checkpoint inhibitor that targets the Programmed Cell Death Protein 1 (PD-1) pathway; administering to the subject a composition comprising an INKT cell modulator, such as IMM60, in an amount effective to increase sensitivity of the cancer to the immune checkpoint inhibitor; and then administering to the subject a composition comprising the immune checkpoint inhibitor following the administration of the composition comprising IMM60. In some variations, the subject may be tested/evaluated following the administration of the INKT modulator, to determine whether the subject should no longer be scored as “insensitive.” For example, a subject may no longer be scored as insensitive because a biomarker, such as PD-L1 expression, has changed, and the subject now qualifies for treatment under prescribing guidelines for the checkpoint inhibitor. For the subject identified as sensitive, the checkpoint therapy is initiated (or resumed)—either alone or in combination with other standard of care therapy for the cancer. In some variation, the subject continues to be treated with INKT cell modulator, in combination with the checkpoint inhibitor.

In some embodiments, such as embodiments that use one or more biomarkers for evaluating sensitivity/insensitivity, a threshold or cut-off value is used to score subjects as sensitive or insensitive. The cut-off may be a single number for a single biomarker, such as a percentage of cells that are positive for a marker such as PD-1, PD-L1, or PD-L2. The cut-off may be derived or calculated from multiple measurements, e.g., when multiple biomarkers or multiple cell types are considered. In some embodiments, the cut-off may be derived from clinical trial patient information or extrapolated from pre-clinical animal models. In some embodiments, the threshold or cut-off value is from prescribing information for a particular drug. In some embodiments, a statistical analysis from multiple patient outcomes, such as a receiver operating characteristic (ROC) curve, is used to optimize a threshold or cutoff.

In some embodiments, the INKT cell modulator, such as IMM60, is formulated in a liposomal composition, such as the liposomal compositions described herein. In some embodiments, the IMM60 is formulated in a nanoparticle formulation.

Exemplary embodiments of the invention are summarized or defined by the following numbered paragraphs:
201. A method of treating a mammalian subject with an immune checkpoint inhibitor, the method comprising:
(a) identifying a mammalian subject as having a cancer that is insensitive to treatment with an immune checkpoint inhibitor that targets the Programmed Cell Death Protein 1 (PD-1) pathway;
(b) administering to the subject a composition comprising IMM60, in an amount effective to increase sensitivity of the cancer to the immune checkpoint inhibitor; and
(c) administering to the subject a composition comprising the immune checkpoint inhibitor following the administration of the composition comprising IMM60.
202. The method according to paragraph 201, that comprises identifying the subject as insensitive to the checkpoint inhibitor by:
(a1) measuring expression of Programmed Cell Death Ligand 1 (PD-L1) in cancer cells, immune cells, or tumor stroma cells from a tumor sample from the subject; and
(a2) identifying the subject as having a cancer that is insensitive to treatment with the immune checkpoint inhibitor by determining that the target of the checkpoint inhibitor is expressed on a percentage of the cancer cells that is lower than a threshold or cut-off level.
203. The method according to paragraph 201, that comprises identifying the subject as insensitive to the checkpoint inhibitor by:
(a1) measuring microsatellite instability (MSI), mismatch repair deficiency (MMRD), or tumor mutational burden (TMB) in cancer cells from the subject, and
(a2) identifying the mammalian subject as having a cancer that is insensitive to treatment with the immune checkpoint inhibitor by determining that the cancer cells have an MSI, MMRD, or TMB that is lower than a threshold or cut-off level.
204. The method according to paragraph 201, that comprises identifying the subject as insensitive to the checkpoint inhibitor by:
(a1) comparing a medical diagnosis and history of the subject with a government-approved prescribing guideline for the immune checkpoint inhibitor, and
(a2) identifying the mammalian subject as having a cancer that is insensitive to treatment with the immune checkpoint inhibitor by determining that that the subject does not satisfy the indication and usage criteria of the guideline.
205. The method according to paragraph 204, wherein the prescribing guideline specifies a minimum expression level of PD-L1 in cells from the subject, and (a2) comprises determining that the cells from the subject fail to meet the minimum expression level.
206. The method according to paragraph 204 or 205, wherein the immune checkpoint inhibitor was a United States Food and Drug Administration (FDA)-approved immune checkpoint inhibitor before or in 2022, and the prescribing guideline is the FDA approved guideline in existence on Dec. 31, 2022.
207. The method according to paragraph 204 or 205, wherein
the immune checkpoint inhibitor was a European Medicines Agence (EMA)-approved immune checkpoint inhibitor before or in 2022, and the prescribing guideline is the EMA-approved guideline in existence on Dec. 31, 2022; or
wherein the immune checkpoint inhibitor was a Japanese Pharmaceuticals and Medical Devices Agency (PDMA)-approved immune checkpoint inhibitor before or in 2022, and the prescribing guideline is the PDM-approved guideline in existence on Dec. 31, 2022; or
wherein the immune checkpoint inhibitor was a Chinese National Medical Products Administration (NMPA)-approved immune checkpoint inhibitor before or in 2022, and the prescribing guideline is the NMPA-approved guideline in existence on Dec. 31, 2022.
208. The method according to paragraph 201, that comprises identifying a patient as insensitive to the checkpoint inhibitor by:
(a1) comparing a medical diagnosis and history of the subject with a standard-of-care guideline for use of the immune checkpoint inhibitor; and
(a2) identifying the subject as having a cancer that is insensitive to treatment with the immune checkpoint inhibitor by determining that the subject does not meet the standard-of-care guideline.
209. The method according to paragraph 208, wherein the standard-of-care guideline specifies a minimum expression level of PD-L1 in cells from the subject, and (a2) comprises determining that the cells from the subject fail to meet the minimum expression level.
210. The method according to any one of paragraphs 201 to 209, wherein (b) further comprises:
(b1) re-evaluating a sample from the subject after one or more administrations of the IMM60; and
(b2) determining from the sample that the cancer is no longer classified as insensitive to the treatment with the immune checkpoint inhibitor.
211. The method according to paragraph 201, wherein the mammalian subject is a subject that has previously received the immune checkpoint inhibitor and been diagnosed as resistant to the immune checkpoint inhibitor prior to the administering of the IMM60.
212. The method according to any one of paragraphs 201 to 211, wherein step (c) comprises combination therapy, the combination comprising administration of the checkpoint inhibitor and continued administration of IMM60 to the subject.
213. The method according to paragraph 212, wherein step (c) comprises repeated administrations of the composition comprising IMM60 and the composition comprising the immune checkpoint inhibitor to the subject.
214. The method according to paragraph 212 or 213, further wherein the combination therapy further comprises administering a standard-of-care chemotherapy for the cancer to the subject.
215. A method of sensitizing a cancer in a mammalian subject to treatment with an immune checkpoint inhibitor, the method comprising:
identifying a subject as having a cancer that is insensitive to treatment with an immune checkpoint inhibitor that targets the Programmed Cell Death Protein 1 (PD-1) pathway; and
administering to the subject a composition comprising IMM60 in an amount and for a duration effective to sensitize the cancer to treatment with the immune checkpoint inhibitor.
216. The method according to paragraph 215, wherein the identifying comprises:
measuring expression of Programmed Cell Death Ligand 1 (PD-L1) in cancer cells, immune cells, or tumor stroma cells from a tumor sample from the subject; and
identifying the mammalian subject as having a cancer that is insensitive to treatment with the immune checkpoint inhibitor by determining that the PD-L1 is expressed on a percentage of the cells that is lower than a threshold or cut-off level.
217. The method according to paragraph 216, wherein the threshold or cut-off level comprises a level specified in a government-approved prescribing guideline for the immune checkpoint inhibitor.
218. The method according to paragraph 217, wherein the immune checkpoint inhibitor was a United States Food and Drug Administration (FDA)-approved immune checkpoint inhibitor before or in 2022, and the prescribing guideline is the FDA approved guideline in existence on Dec. 31, 2022.
219. The method according to paragraph 217, wherein:
the immune checkpoint inhibitor was a European Medicines Agency (EMA)-approved immune checkpoint inhibitor before or in 2022, and the prescribing guideline is the EMA-approved guideline in existence on Dec. 31, 2022; or
wherein the immune checkpoint inhibitor was a Japanese Pharmaceuticals and Medical Devices Agency (PDMA)-approved immune checkpoint inhibitor before or in 2022, and the prescribing guideline is the PDM-approved guideline in existence on Dec. 31, 2022; or
wherein the immune checkpoint inhibitor was a Chinese National Medical Products Administration (NMPA)-approved immune checkpoint inhibitor before or in 2022, and the prescribing guideline is the NMPA-approved guideline in existence on Dec. 31, 2022.
220. The method according to paragraph 216, wherein the threshold or cut-off level comprises a level specified in a standard-of-care guideline for use of the immune checkpoint inhibitor for the subject's cancer.
221. The method according to any one of paragraphs 215-220, further comprising:
determining that the cancer has become a candidate for treatment with the immune checkpoint inhibitor, subsequent to at least one administration of the composition comprising the IMM60, from an increase in expression of PD-L1 in cancer cells of the subject.
222. The method according to paragraph 221, wherein the determining comprises:
measuring expression of PD-L1 in cancer cells, immune cells, or tumor stroma cells from a tumor sample of the subject subsequent to at least one administration of the composition comprising the IMM60; and
identifying the mammalian subject as having a cancer that has become a candidate for treatment with the immune checkpoint inhibitor from a measurement of PD-L1 on a percentage of the cells that is equal to or greater than the threshold or cut-off level.
223. The method according to paragraph 222, wherein the threshold or cut-off level is expression of PD-L1 on 1% of the cancer cells, or 5% of the cancer cells, or 10% of the cancer cells, or 25% of the cancer cells, or 50% of the cancer cells assayed for PD-L1 expression.
224. The method according to paragraph 222, wherein the mammalian subject is identified as a candidate from an increase in expression is an increase of at least 10%, or at least 20%, or at least 25%, or at least 50%, or at least 100%, or at least 200%, or at least 500% in the percentage of cells that express PD-L1 subsequent to the administration of the composition comprising the IMM60, compared the percentage of cells that expressed PD-L1 prior to the administration.
225. The method according to any one of paragraphs 221 to 224, further comprising administering a composition comprising the immune checkpoint inhibitor to the subject determined to have the cancer that has become a candidate for treatment with the immune checkpoint inhibitor.
226. The method according to any one of paragraphs 202-203 and 216-225, wherein the measuring comprises a PD-L1 immunoassay.
227. A method of cancer treatment comprising:
administering, to a mammalian subject having a PD-L1-negative cancer, a composition comprising IMM60 in an amount effective to stimulate PD-L1 expression in cancer cells; and
administering to the subject a composition comprising an immune checkpoint inhibitor that targets the Programmed Cell Death Protein 1 (PD-1) pathway.
228. The method of paragraph 227 that comprises:
administering, to a mammalian subject having a PD-L1-negative cancer, a composition comprising IMM60;
determining that cancer in the subject has become PD-L1-positive following at least one administration of the IMM60; and
administering to the subject the composition comprising the immune checkpoint inhibitor that targets the Programmed Cell Death Protein 1 (PD-1) pathway after determining that cancer in the subject has become PD-L1 positive.
229. The method of paragraph 227 or 228, further comprising measuring PD-L1 in cancer cells from the subject prior to initiating the administering of the composition comprising IMM60, and determining that PD-L1 expression in the cancer cells is below a threshold for classifying the cancer cells as PD-L1-negative.
230. The method according to any one of paragraphs 226-229, further comprising measuring PD-L1 in cancer cells from the subject subsequent to initiating the administering of the composition comprising IMM60, and determining that PD-L1 expression is above a threshold or cut-off for classifying the cancer cells as PD-L1-negative.
231. A composition comprising IMM60 for use in increasing the expression of PD-L1 in cancer cells of a subject identified as having a PD-L1-negative cancer.
232. The composition of paragraph 231 for use in an ordered combination treatment regimen for cancer, said treatment regimen further comprising subsequent use of a composition comprising an immune checkpoint inhibitor that targets the Programmed Cell Death Protein 1 (PD-1) pathway.
233A. IMM60 and a PD-L1 inhibitor for use as a combination therapy for treatment of cancer in a subject having a PD-L1-negative cancer.
233B. IMM60 and a PD-1 or PD-L1 inhibitor for use as a combination therapy for treatment of cancer in a subject having a cancer that is insensitive to the PD-1 or PD-L1 inhibitor.
234. The method or use according to any one of paragraphs 202-214, 216-226, and 229-230, wherein the threshold or cut-off for determining that a cancer is insensitive to PD-L1 therapy is PD-L1 expression on less than 1% of cancer cells assayed for PD-L1 expression, or less than 5% of cancer cells assayed for PD-L1 expression, or less than 10% of cancer cells assayed for PD-L1 expression, or less than 25% of cancer cells assayed for PD-L1 expression or less than 50% of cancer cells assayed for PD-L1 expression.
235. The method or use according to any one of paragraphs 202-214, 216-226, and 229-230, wherein the threshold or cut-off level comprises a level specified in a government-approved prescribing guideline for the immune checkpoint inhibitor.
236. The method according to any one of paragraphs 202-214, 216-226, and 229-230, wherein the anti-PD-L1 therapy is a checkpoint inhibitor is a United States Food and Drug Administration (FDA)-approved immune checkpoint inhibitor approved before or in 2022, and the threshold or cut-off is specified in the FDA approved guideline in existence on Dec. 31, 2022.
237. The method or use according to any preceding paragraph, wherein the subject is human.
238. The method or use according to any preceding paragraph, wherein the composition comprising IMM60 is a liposomal composition.
239A. The method according to paragraph 238, wherein the IMM60 is administered at a dose of 3-9 mg/m².
239B. The method according to paragraph 238, wherein the IMM60 is administered at a dose of 30-36 mg.
240. The method according to any of paragraphs 238-239B, wherein IMM60 is administered every three weeks for six cycles.
241. The method or use according to any preceding paragraph, wherein the immune checkpoint inhibitor is an antibody that binds to PD-1 or an antibody that binds to PD-L1.
242. The method according to paragraph 241, wherein the immune checkpoint inhibitor comprises Pembrolizumab, Nivolumab, or Cemiplimab.
243. The method according to paragraph 241, wherein the immune checkpoint inhibitor comprises Atezolizumab, Avelumab, or Durvalumab.
244. The method or use according to any preceding paragraph, wherein the cancer comprises melanoma, non-small cell lung cancer, colorectal cancer, head and neck cancer, renal cell carcinoma, Hodgkin's lymphoma, squamous cell carcinoma, Merkel cell cancer, cervical cancer, endometrial cancer, Primary mediastinal large B-cell lymphoma (PMBCL), basal cell carcinoma, biliary track cancer, bladder cancer, breast cancer, esophageal cancer, and mesothelioma.
245. The method or use according to any preceding paragraph, wherein the cancer is non-small cell lung cancer or melanoma.
246. The method or use according to any one of paragraphs 201-243, wherein the subject has received chemotherapy, e.g., platinum-based chemotherapy, or immunotherapy prior to initiating IMM60 therapy.

The foregoing summary is not intended to define every aspect of the disclosure, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the disclosure includes, as an additional aspect, all embodiments of the disclosure narrower in scope in any way than the variations specifically mentioned above. With respect to aspects of the disclosure described or claimed with “a” or “an,” these terms mean “one or more” unless context unambiguously requires a more restricted meaning. With respect to elements described as one or more within a set, all combinations within the set are contemplated as combination inventions. If aspects of the disclosure are described as “comprising” a feature, embodiments also are contemplated “consisting of” or “consisting essentially of” the feature.

Aspects of the disclosure described as methods of treatment should also be understood to include first or subsequent “medical use” aspects of the disclosure or “Swiss use” of compositions for the manufacture of a medicament for treatment of the same disease or condition.

Multiple embodiments are contemplated for combinations described herein. For example, some aspects of the disclosure that are described as a method of treatment (or medical use) combining two or more compounds or agents, whether administered separately (sequentially or simultaneously) or in combination (co-formulated or mixed). For each aspect described in this manner, the disclosure further includes a composition comprising the two or more compounds or agents co-formulated or in admixture with each other; and the disclosure further includes a kit or unit dose containing the two or more compounds/agents packaged together, but not in admixture. Optionally, such compositions, kits or doses further include one or more carriers in admixture with one or both agents or co-packaged for formulation prior to administration to a subject. The reverse also is true: some aspects of the disclosure are described herein as compositions useful for therapy and containing two or more therapeutic agents. Equivalent methods and uses are specifically contemplated.

The disclosure also pertains to materials and method for improving cancer therapy with checkpoint inhibitors, including materials and methods that expand the patients or cancers that are treatable with checkpoint inhibitors and/or that improve the efficacy of treatment.

Although the applicant(s) invented the full scope of the claims appended hereto, the claims appended hereto are not intended to encompass within their scope the prior art. Therefore, in the event that statutory or judicially recognized prior art within the scope of a claim is brought to the attention of the applicants by a Patent Office or other entity or individual, the applicant(s) reserve the right to exercise amendment rights under applicable patent laws to redefine the subject matter of such a claim to specifically exclude such prior art or obvious variations of statutory prior art from the scope of such a claim. Variations of the disclosure defined by such amended claims also are intended as aspects of the invention. Additional features and variations of the invention will be apparent to those skilled in the art from the entirety of this application, and all such features are intended as aspects of the disclosure.

### BRIEF DESCRIPTION OF THE DRAWINGS

FIG. 1A shows results of an experiment administering 0.1 ng, 1 ng, and 5 ng of IMM60 formulated in liposomal compositions disclosed herein injected intravenously into wildtype C57 BL/or C57 BL/CD1d KO −/− (NKT cell-deficient) mice, wherein the IFNγ levels were measured in serum 18 hours post-injection.

FIG. 1B shows the results of an experiment administering 0.1 ng, 1 ng, and 5 ng of IMM60 formulated in liposomal compositions disclosed herein injected intravenously into wildtype C57 BL/or C57 BL/CD1d KO −/− (NKT cell-deficient) mice, wherein the CD8 levels on dendritic cells (CD11c⁺ MHC-class II⁺) were measured.

FIG. 1C shows the results of an experiment administering 0.1 ng, 1 ng, and 5 ng of IMM60 formulated in liposomal compositions disclosed herein, and in soluble form (5 ng), injected intravenously into wildtype C57 BL/or C57 BL/CD1d KO −/− (NKT cell-deficient) mice, wherein the PD-L1 levels on dendritic cells (CD11c⁺ MHC-class II+) were measured.

FIG. 1A-1C abbreviations: DC=dendritic cell; Geo MCF=geometric mean channel fluorescence; IFN-7=interferon-gamma; KO=knockout; MHC=major histocompatibility complex; PD-L1=programmed cell death receptor ligand 1.

FIG. 1D shows the results of an experiment administering 0.1 ng, 1 ng, and 5 ng of IMM60 formulated in liposomal compositions disclosed herein, injected intravenously into wildtype C57 BL/(ten bars on left) or C57 BL/CD1d KO −/− (NKT cell-deficient) mice (four bars on right), wherein the CD40 levels on dendritic cells (CD11c⁺ MHC-class II⁺) were measured. The CD40 levels of mice that were administered the compositions disclosed herein are compared to the CD40 levels of mice that were administered solubilized IMM60 (sol IMM60) not formulated in liposomes.

FIG. 1E shows the results of an experiment administering 0.1 ng, 1 ng, and 5 ng of IMM60 formulated in liposomal compositions disclosed herein intravenously injected into wildtype C57 BL/(ten bars on left) or C57 BL/CD1d KO −/− (NKT cell-deficient) mice (four bars on right), wherein the CD8 levels on B cells were measured. The CD8 levels of mice that were administered the compositions disclosed herein are compared to the CD8 levels of mice that were administered solubilized IMM60 (sol IMM60) not formulated in liposomes.

FIG. 1F shows the results of an experiment administering 0.1 ng, 1 ng, and 5 ng of IMM60 formulated in liposomal compositions disclosed herein intravenously injected into wildtype C57 BL/(ten bars on left) or C57 BL/CD1d KO −/− (NKT cell-deficient) mice (four bars on right), wherein the PDL-1 levels on B cells were measured.

FIGS. 2-4 summarize the results of experiments involving the administering of 0.01 ng, 0.1 ng, and 1 ng of IMM60 formulated in liposomal compositions disclosed herein injected intravenously into wildtype C57 BL/or C57 BL/CD1d KO −/− (NKT cell-deficient) mice, wherein the levels of IFNγ, IL-12p70, IL-2, IL-1β, IL6, KC/GRO, TNF-α, IL-10, IL-4, and IL-5 were measured at 4 hours, 18 hours, and 72 hours post-injection. The levels of these cytokines and markers following injection of liposomal compositions disclosed herein are compared to levels following injection of solubilized IMM60 (IMM60 control, not formulated in liposomes) or vehicle (Veh) control. FIG. 2 summarizes data from experiments performed with IMM60 formulated in POPC/DDAB liposomes. FIG. 3 summarizes data from experiments performed with IMM60 formulated in DSPC/DSPG liposomes. FIG. 4 summarizes data from experiments performed with IMM60 formulated in POPC/DC-Chol liposomes.

FIG. 5 shows the results of an experiment of (from left to right for each formulation) 10 ng, 100 ng, 1000 nm and 5000 ng of IMM60 formulated in liposomal compositions disclosed herein intravenously injected into wildtype C57 BL/6, wherein the levels of IFNγ were measured in serum 18 hours post-injection.

Figure shows the results of an experiment in which mice were treated with soluble IMM60 or a liposomal IMM60 formulation as disclosed herein. IFN-γ was measured 18 hours post-treatment.

FIG. 7 shows measurements of CD8 on dendritic cells in mice that were treated with a liposomal IMM60 formulation as disclosed herein compared to mice treated with soluble (free) IMM60 or vehicle-treated or untreated controls.

FIG. 8 shows measurements of antigen-specific T cells in mice seven days after treatment with an IMM60 liposomal (lip) formulation as disclosed herein with an ovalbumin peptide antigen compared to soluble IMM60 with the antigen.

FIG. 9A shows the results of a prophylactic dose-response experiment to evaluate the ability of DPSC/DSPG/IMM60 liposomes to inhibit metastatic nodules in the lungs. Mice were injected with the liposomal IMM60 formulation at the specified concentrations three days before injection with cells from a melanoma tumor cell line. FIG. 9B shows the results of a similar, therapeutic dose-response experiment. Mice were injected with the liposomal IMM60 formulation at the specified concentrations three days after injection with the cells from the melanoma tumor cell line. Abbreviations: NS=not significant; *=P<0.05; **=P<0.01; ***=P<0.001.

FIG. 10 shows the results of a dose-response experiment in mice treated with an IMM60 liposomal formulation disclosed herein, alone or in combination with anti-PD-1 antibodies, in a separate subcutaneous melanoma mouse model. IFN-γ was measured 18 hours following the first treatment dose. Abbreviations: Ab=antibody; IFNγ=interferon-gamma; PD-1=programmed cell death protein-1.

FIG. 11 shows the results of tumor growth experiment in mice treated with the liposomal formulation disclosed herein, anti-PD-1 blocking antibodies, or both, in a subcutaneous melanoma mouse model.

FIGS. 12A and 12B show the results of longitudinal tumor growth kinetics in the subcutaneous CT-2 tumor model in mice treated with liposomal formulation disclosed herein, anti-PD-1 blocking antibodies, or both. FIG. 12C shows the results at day 14 of longitudinal tumor growth kinetics in the subcutaneous CT-2 tumor model in mice treated with liposomal formulation disclosed herein, anti-PD-1 blocking antibodies, or both. Abbreviations: IFN=interferon; PD-1=programmed cell death protein-1. FIG. 12D shows measurements of interferon gamma in blood drawn from the mice at t=18 hours in the longitudinal study.

FIG. 13 depicts the results of a study (Example 9) in which PDL-1 expression was measured on Melanoma B16f10 cells following exposure of the cells to single agents and agent combinations at different concentrations.

FIG. 14 depicts the effect of IMM60 at different concentrations, alone or in combination with anti-PD-1 antibody, on T cells in B16f10:splenocyte cocultures. (Example 9.)

FIGS. 15A and 15B depict the schema for Phase 1 and Phase 2 clinical protocols in Example 13.

FIGS. 16-25 depict preliminary results from the ascending dose arm of the clinical protocol in Example 12 described herein. FIG. 16 shows the 24 h exposure results from the human pharmacokinetics study described in Example 12.

FIG. 17 shows the exposure data from FIG. 16 normalized for dose group.

FIG. 18 shows the total exposure results in AUC_{∞} from the human pharmacokinetics study described in Example 12.

FIG. 19 shows the total exposure results in AUC_{∞} normalized for actual dose from the human pharmacokinetics study described in Example 12.

FIG. 20 shows the relative exposure in AUC_{∞} by dose group from the human pharmacokinetics study described in Example 12.

FIG. 21 shows the Cₘₐₓ by dose group from the human pharmacokinetics study described in Example 12.

FIG. 22 shows the terminal half-life by dose group from the human pharmacokinetics study described in Example 12.

FIG. 23 shows the total exposure in AUC_{∞} by dose group normalized by actual dose from the human pharmacokinetics study described in Example 12.

FIG. 24 shows the Cₘₐₓ by dose group normalized by actual dose from the human pharmacokinetics study described in Example 12.

FIG. 25 shows the mean residence time (MRT) by dose group from the human pharmacokinetics study described in Example 12.

### DETAILED DESCRIPTION

Disclosed herein are liposomes that include the following Compound A,

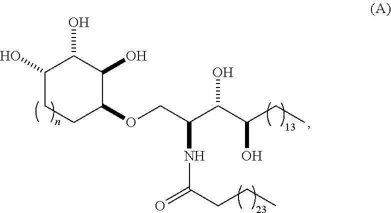

wherein n is 1 (IMM60), 2 (IMM70), or 3 (IMM80), or a salt, ester, solvate, or hydrate thereof, and two or more lipids or salts thereof. Also disclosed are compositions that comprise the liposomes and a pharmaceutically acceptable diluent, excipient, adjuvant, or carrier.

Liposomes provided herein provide for a more concentrated and potent delivery form for Compound A; and provide for stability of the compound. Additionally, the liposomal formulation is contemplated to improve absorption, distribution, metabolism and/or elimination (ADME) of Compound A, especially for treatment of cancer tumors. Without intending to be limited to any particular theory, some of the advantages of liposomal formulation described herein include an EPR effect (enhanced permeability and retention), a longer in vivo half-life, a lower volume of distribution, and better toleration (reduction in adverse events).

Compound A (iNKT Modulators) or a Salt, Ester, Solvate, or Hydrate Thereof

The compounds categorized herein as Compound A and methods of making them are described in International Patent Application No. PCT/EP2012/074140, filed 30 Nov. 2012, published as International Publication No. WO 2013/079687, “INKT Cell Modulators and Methods of Using the Same,” incorporated herein by reference in its entirety. See also U.S. Pat. No. 9,365,496, incorporated herein by reference.

The salts, e.g., pharmaceutically acceptable salts, of the disclosed therapeutics may be prepared by reacting the appropriate base or acid with a stoichiometric equivalent of the therapeutic.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and those formed with organic acids such as maleic acid.

Pharmaceutically acceptable base addition salts may be formed with metals or amines, such as alkali and alkaline earth metals or organic amines. Pharmaceutically acceptable salts of Compound A may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Similarly, pharmaceutically acceptable derivatives (e.g., esters), metabolites, hydrates, solvates and prodrugs of the therapeutic may be prepared by methods generally known to those skilled in the art. Thus, another embodiment provides compositions that include prodrugs of active Compound A. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of the therapeutic. As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than carbon atoms. Representative examples of particular esters include, but are not limited to, formates, acetates, propionates, butyrates, acrylates and ethylsuccinates. Examples of pharmaceutically-acceptable prodrug types are described in Higuchi and Stella, Pro-drugs as Novel Delivery Systems, Vol. 14 of the A.C.S. Symposium Series, and in Roche, ed., Bioreversible Carriers in Drug Design, American Pharmaceutical Association and Pergamon Press, 1987, both of which are incorporated herein by reference.

The compositions including Compound A described herein may also include metabolites. As used herein, the term “metabolite” means a product of metabolism of Compound A or a pharmaceutically acceptable salt, analog, or derivative thereof, that exhibits a similar activity in vitro or in vivo to a disclosed therapeutic. The compositions described herein may also include hydrates and solvates. As used herein, the term “solvate” refers to a complex formed by a solute (herein, the therapeutic) and a solvent. Such solvents for the purpose of the embodiments preferably should not negatively interfere with the biological activity of the solute. Solvents may be, by way of example, water, ethanol, or acetic acid.

The compositions disclosed herein can stimulate an immune response in a subject, e.g., a mammalian subject. In various cases, the compositions stimulate iNKT cells in a subject. In various cases, the compositions stimulate an immune response, as measured by an in vitro assay. In various cases, the compositions stimulate an immune response in a subject in vivo.

### Liposomes and Liposomal Formulations

Described herein are liposomal formulations of iNKT modulators such as those referred to herein as Compound A. Also described herein are liposomes comprised of a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent, wherein the phospholipid bilayer is substantially devoid of cholesterol, plant sterols, and polyethylene glycolated lipids (PEG lipids). As used herein, “polyethylene glyocolated lipid” refers to a lipid that has been chemically modified (e.g., via conjugation) to comprise a PEG moiety or PEG functional group(s).

Liposomes are lipid-containing vesicles that include one, two, or several concentric lipid bilayers (typically comprised of phospholipids) enclosing an aqueous core, and can be used to deliver both hydrophilic and hydrophobic active pharmaceutical ingredients (APIs) to the body. Small, hydrophilic active pharmaceutical ingredients can be encapsulated within the aqueous core (cavity) of a liposome, e.g., in solution or suspension. Both charged, hydrophilic active pharmaceutical ingredients and uncharged, hydrophobic active pharmaceutical ingredients can be associated with the membrane of a liposome through electrostatic or hydrophobic interactions, respectively, or through a covalent bond. Liposomes can deliver active pharmaceutical ingredients to the body by, for example, fusion of the liposome with other bilayers, such as the cell membrane, diffusion of the compound out of the liposome, or digestion of the liposome within macrophages.

Numerous methods for liposomal manufacture are known, typically involving applying some form of energy to a dispersion of phospholipids in a polar solvent. Exemplary methods include or involve sonication, extrusion, microfluidization, and heating. See, e.g., Panahi et al., Artificial Cells, Naonmedicine, and Biotechnology, 45:4, 788-799 (2017), incorporated herein by reference. All liposomal manufacturing methodologies are contemplated as embodiments for manufacturing the liposomes described herein.

In some embodiments, the disclosure provides a method or preparing a liposome comprising a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent, wherein the phospholipid bilayer is substantially devoid of cholesterol, plant sterols, and polyethylene glycolated lipids (PEG lipids), wherein the method comprises 1) admixing the therapeutic agent and the at least two phospholipids to form multi-lamellar vesicles and 2) extruding the multi-lamellar vesicles through a membrane to form the liposomes.

The disclosed methods comprise extruding the multi-lamellar vesicles through a membrane to form the liposomes. The membrane has a suitable pore size (e.g., about 50 nm to about 250 nm, about 50 nm to about 150 nm, or about 60 nm to about 100 nm, or about 80 nm).

In some embodiments, the disclosed methods further comprise purifying the liposomes. The liposomes can be purified using any suitable technique. For example, in some embodiments the liposomes are purified using filtration techniques. Suitable filtration techniques include, for example, ultrafiltration, centrifugation, dialysis, diafiltration, or tangential flow filtration (TFF). In embodiments comprising purifying the liposome using filtration, the liposomes are filtered using a suitable pore size. Suitable pore sizes include, for example, a pore size of about 450 nm or less, or about 400 nm or less, about 350 nm or less, or about 300 nm or less, or about 250 nm or less, or about 220 nm or less, or about 200 nm or less, or about 150 nm or less. In some embodiments, the filter has a molecular weight cut off (MWCO) of about 500 kD or less (e.g., about 450 kD, about 400 kD, about 350 kD, or about 300 kD, or about 250 kD).

As described in U.S. Pat. No. 7,060,291, incorporated herein by reference, liposomes can have a variety of sizes, e.g., an average diameter as low as 25 nm or as high as 10,000 nm or more. Size is affected by a number of factors, such as, for example, lipid composition and method of preparation, and is determined by a number of techniques, such as dynamic light scattering (DLS). Various methodologies can be used to prepare liposomes of a smaller size from larger liposomes, such as sonication, homogenization, French Press application, and milling. Extrusion (see, e.g., U.S. Pat. No. 5,008,050, incorporated herein by reference) can be used to produce liposomes having a predetermined mean size by forcing the liposomes, under pressure, through filter pores of a defined, selected size. Tangential flow filtration, as described in PCT Application No. WO 1989/008846, incorporated herein by reference, also can be used to regularize the size of liposomes (i.e., produce a population of liposomes having less size heterogeneity, and a more homogeneous, defined size distribution).

Using liposomes to deliver an active pharmaceutical ingredient (API) or ingredients, such as IMM60, IMM70, or IMM80, is highly advantageous. Unlike micellar delivery vehicles, liposomes are physically stable. The liposomal lipid composition can be tailored to deliver specific APIs or therapeutic agents to targeted locations, which limits the potential toxicity of the ingredients. Further, liposomes allow the delivery of known amounts of APIs because they minimize loss of the API from, for example, degradation, removal by non-target organs, and precipitation, as seen with detergents. Liposomes also provide a method for co-delivering adjuvants and antigens in particular ratios that otherwise cannot occur by, for example, coinjection, as described in U.S. Pat. Nos. 7,850,990 and 7,842,676, each incorporated herein by reference.

In one aspect, described herein is a composition including a liposome, wherein the liposome comprises Compound A or a salt, ester, solvate, or hydrate thereof and two or more lipids, and a pharmaceutically acceptable diluent, excipient, adjuvant, or carrier. In embodiments, the liposome includes a lipid bilayer membrane surrounding an aqueous core, wherein the lipid bilayer membrane comprising two or more lipids (e.g., 2, 3, 4, 5, 6, 7, or 8 lipids).

Specifically contemplated are liposomes comprised of Compound A and two or more lipids; exemplary pairs of lipids for making the liposomes include:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); and
(f) 1,2-dimyristoyl-sn-glycero-3-phosphoglycerol (DMPG) and cholest-5-en-30-ol (CHOL).

The disclosure also contemplates liposomes comprising at least one therapeutic agent and at least two phospholipids selected from:
1,2-distearoyl-sn-glycero-3-phosphocholine (DSPC);
1,2-distearoyl-sn-glycero-3-phospho-rac-glycerol (DSPG);
1,2-dipalmitoyl-sn-glycero-3-phosphocholine (DPPC); and
1,2-dipalmitoyl-sn-glycero-[phosphor-rac-(1-glycerol)](DPPG), wherein
the phospholipid bilayer is substantially devoid of cholesterol, plant sterols, and polyethylene glycolated lipids (PEG lipids).

In some embodiments, the disclosed liposomes may comprise at least two (e.g., 3) phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG). In some embodiments, the phospholipids of the liposomes disclosed herein consist essentially of DSPC and DSPG.

In various embodiments, the liposome comprises a phospholipid bilayer that is substantially devoid of cholesterol, plant sterols, and polyethylene glycolated lipids (PEG lipids). As used herein, the term “substantially” (e.g., “substantially devoid” or “substantially cleared”) refers to little or none of the recited components being present (e.g., 2 wt % or less present, 1.5 wt % or less, 1.0 wt. % or less, 0.5 wt % or less, 0.4 wt % or less, 0.3 wt % or less, 0.2 wt % or less, or 0.1 wt % or less present).

In some embodiments, the disclosed liposomes comprise a phospholipid bilayer having a phase transition temperature above about 37° C. In some embodiments, the disclosed liposomes are free of phospholipids having a phase transition temperature below about 37° C. Moreover, in some embodiments, the disclosed liposomes are free of unsaturated lipids or unsaturated phospholipids.

In some embodiments, the disclosed liposomes are free of lipids or phospholipids having a chain length shorter than 16 carbon atoms. For example, in these embodiments, the liposomes are free of lipids or phospholipids comprising C14, C12, C10 fatty acids, and the like.

The disclosed liposomes substantially devoid of cholesterol, plant sterols, and polyethylene glycolated lipids (PEG lipids) provide many advantages over conventional delivery systems, including other conventional liposomal delivery systems. By way of example, the disclosed liposomes substantially devoid of cholesterol, plant sterols, and PEG lipids provide a delayed release of the payload (e.g., at least one therapeutic agent) and thereby are capable of providing a modified release (e.g., delayed-release, extended-release, sustained-release) of the payload via intravenous infusion. The pharmacokinetic properties when administered to humans demonstrate that the disclosed liposomes provide a Cₘₐₓ over a period of about 1-6 hours (e.g., about 4-6 hours). In addition, the disclosed liposomes substantially devoid of cholesterol, plant sterols, and polyethylene glycolated lipids (PEG lipids) advantageously exhibit a long half-life (e.g., about 6-12 hours) and have relative long residence times in the blood compartment (e.g., 5-10 times longer than other non-PEGylated liposomes) thereby providing improved delivery of the payload and long exposure times to the payload (e.g., higher exposure per dose) to the intended target site (e.g., tumor site).

In some embodiments, the disclosed liposomes substantially devoid of cholesterol, plant sterols, and PEG lipids have a terminal half-life of about 6 hours, about 7 hours, about 8 hours, about 9 hours, about 10 hours, about 11 hours, or about 12 hours. In some embodiments, the liposomes substantially devoid of cholesterol, plant sterols, and PEG lipids have a terminal half-life of about 8 hours.

In some embodiments, the liposomes substantially devoid of cholesterol, plant sterols, and PEG lipids (including compositions comprising the liposomes) have a steady state volume of distribution in a mammalian subject (e.g., human) of about 2 liters to about 12 liters, or about 2 liters to about 10 liters, or about 2 liters to about 8 liters, after being administered to the mammalian subject intravenously. In some embodiments, the liposomes SCD and/or compositions comprising said liposomes have a steady state volume of distribution in a mammalian subject of about 2 liters, about 3 liters, about 4 liters, about 5 liters, about 6 liters, about 7 liters, about 8 liters, about 9 liters, about 10 liters, about 11 liters, or about 12 liters.

In some embodiments, the liposomes substantially devoid of cholesterol, plant sterols, and PEG lipids (including compositions comprising the liposomes) have a terminal volume of distribution in a mammalian subject (e.g., a human) of about 3 liters to about 10 liters (e.g., about 3 liters, about 4 liters, about 5 liters, about 6 liters, about 7 liters, about 8 liters, about 9 liters, or about 10 liters). In some embodiments, the liposomes substantially devoid of cholesterol, plant sterols, and PEG lipids and/or compositions comprising said liposomes have a terminal volume of distribution of about 2 liters to about 9 liters, about 3 liters to about 8 liters, about 4 liters to about 7 liters, or about 5 liters to about 6 liters.

In some embodiments, the liposomes substantially devoid of cholesterol, plant sterols, and PEG lipids (including compositions comprising the liposomes) provide a steady state plasma concentration of therapeutic agent between about 2 hours and about 10 hours (e.g., about 3 h to about 8 h or about 4 h to about 6 h), after administration to a mammalian subject (e.g., human). In another aspect, the therapeutic or active agent delivered by liposomes substantially devoid of cholesterol, plant sterols, and PEG lipids disclosed herein is not substantially cleared from plasma between about 2 hours and about 10 hours, or between about 3 hours to about 8 hours, or between about 4 to about 6 hours, after administration to a mammalian subject (e.g., human). As used herein, “substantially cleared” refers to

The disclosed liposomes further provide an improved safety profile compared to conventional liposomes. When substantially devoid of cholesterol, plant sterols, and polyethylene glycolated lipids (PEG lipids), the disclosed liposomes advantageously exhibit substantially less off-target toxicity even when administered at relative high doses. In some embodiments, the disclosed liposomes do not exhibit any off-target toxicity. In addition, the liposomes disclosed herein substantially devoid of cholesterol, plant sterols, and polyethylene glycolated lipids (PEG lipids), advantageously do not invoke an immune response that can be observed with liposomes comprising (PEG)lipids thereby providing yet a further improved safety profile.

Without wishing to be bound to any particular theory, it is believed that the disclosed liposomes have a size and rigidity that allow them to preferentially pass through “leaky” vasculatures of tumor cells thereby providing greater localization of payload (e.g., anticancer agent) compared to conventional liposomes. In some embodiments, the phenotype of the liposome is “squishy”, e.g., compressible and high(er) membrane fluidity. Moreover, the disclosed liposomes have a higher melting temperature (Tₘ) than conventional liposomes (e.g., about 55° C.) due at least in part to the liposomes comprising saturated lipids.

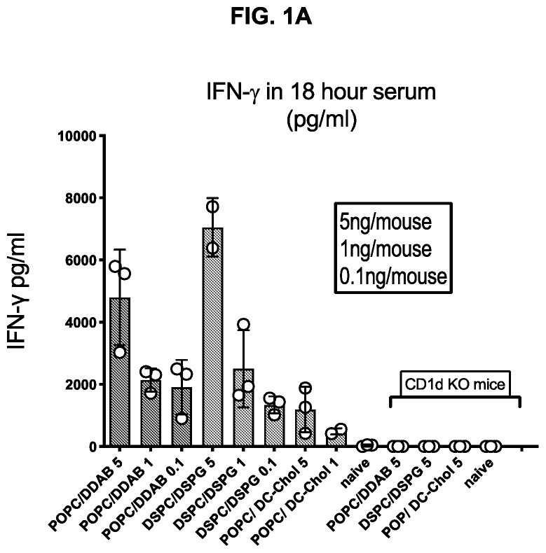

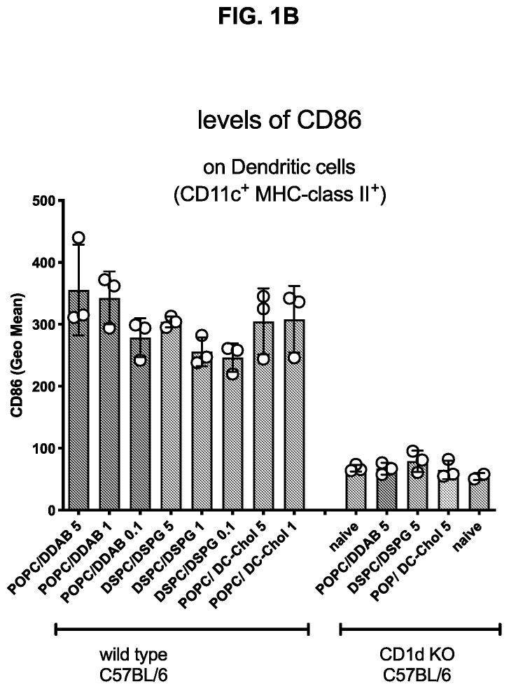

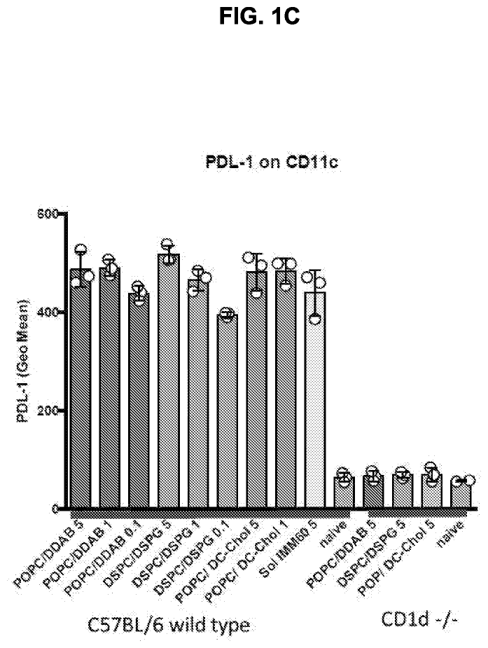

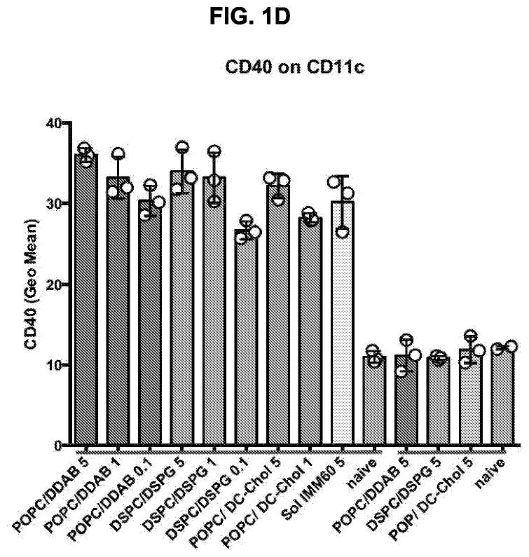

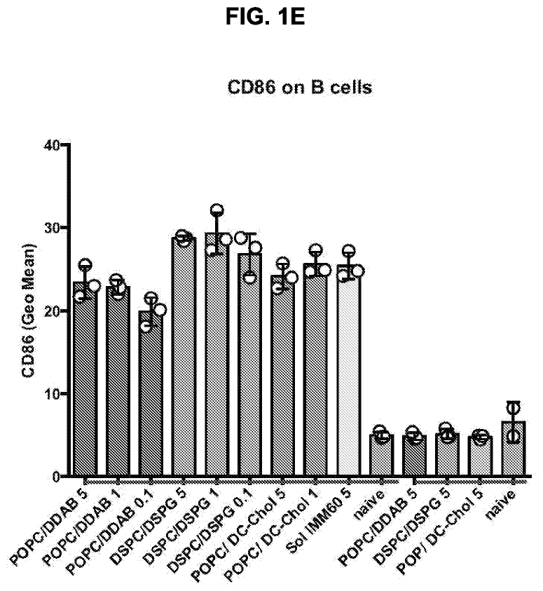

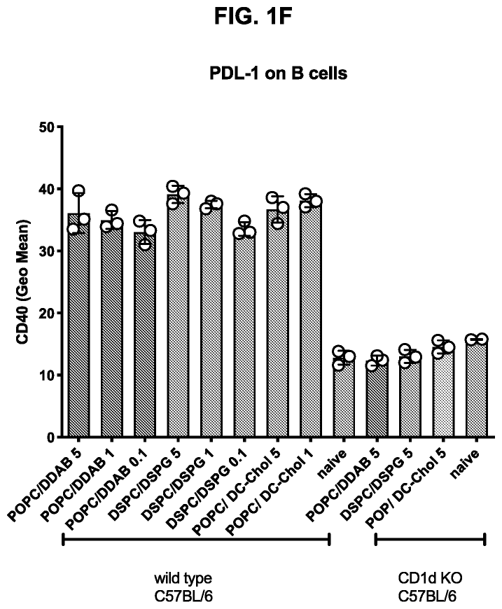

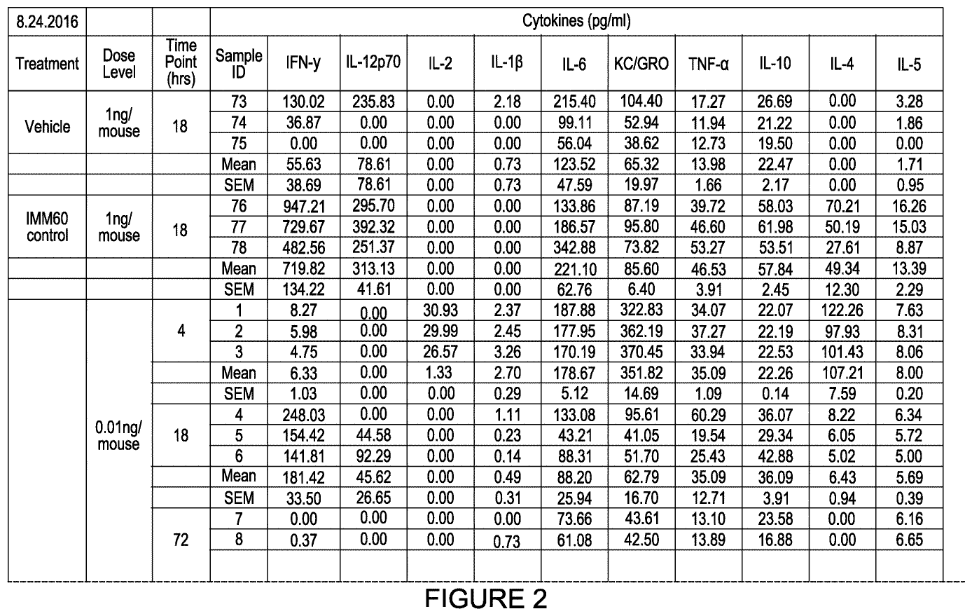

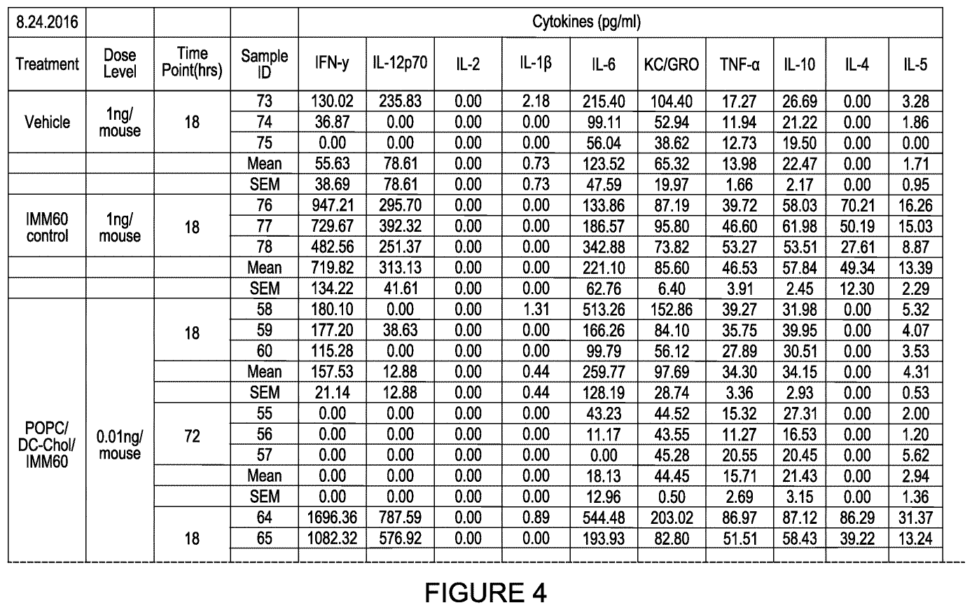

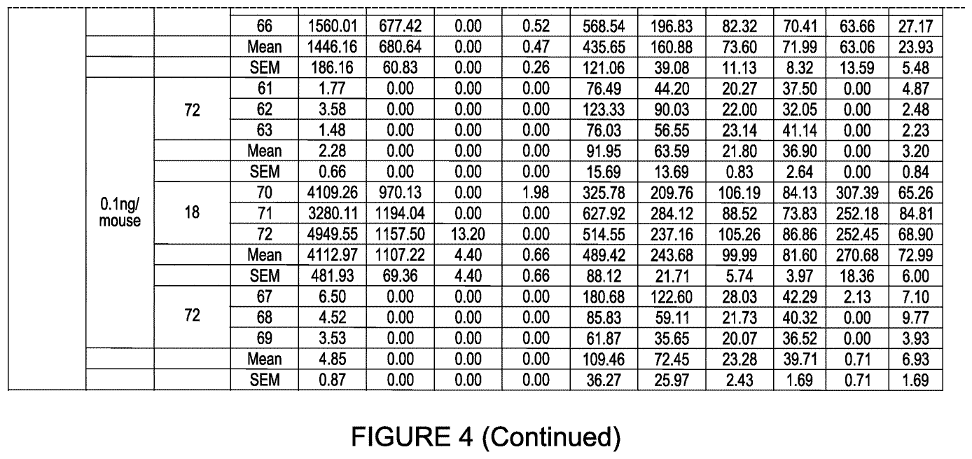

These liposomes provide the desired efficacy of the API (e.g., IMM60) even when the API is used in low concentrations. In experiments described herein, liposomal delivery of IMM60 demonstrated efficacy in laboratory mouse models at various concentrations in the range of, e.g., 0.01 ng/mL to 5000 ng/mL. As shown in FIGS. 1A-1E, 3-5, and 7-8, even low amounts of API delivered (e.g., injected intravenously) in liposomes (e.g., 0.1 ng, 1 ng, and 5 ng) as described herein provide superior efficacy as measurable by a number of relevant biomarkers, whereas the delivery of the solubilized API alone in such low amounts does not provide the same levels of efficacy. As shown in FIGS. 6-8, a dose of 0.1 ng IMM60 formulated in a liposome produced a superior immune response (as measured by multiple markers in a preclinical rodent model) compared to a liposome-free formulation.

In some embodiments, the liposomes in the composition have a mean diameter of less than or equal to about 500 nm, or less than about 400 nm, or less than about 300 nm, or less than about 200 nm, or less than about 150 nm, or less than about 125 nm, wherein the diameter is measured as a Z-average at 90°. Accordingly, in some embodiments the liposomes have a mean diameter within a size range of about 125 nm to about 500 nm, or about 150 nm to about 400 nm, or about 200 nm to about 300 nm.

In some embodiments, the liposomes in the composition have a mean diameter of less than about 200 nm. In some embodiments, the liposomes in the composition have a mean diameter within a size range of about 50 nm to about 200 nm, or about 50 nm to about 170 nm, or about 70 nm to about 130 nm, or about 60 nm to about 160 nm, or about 70 nm to about 150 nm, or about 75 nm to about 145 nm, or about 90 nm to about 130 nm, or about 75 nm to about 145 nm. In some embodiments, the liposomes in the composition have a mean diameter of about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm. In embodiments, the liposomes in the composition can have a mean diameter standard deviation of about 60 nm or less, or about 40 nm, for example, the composition has a mean liposome diameter of about 100 nm±60 nm, of about 100 nm±40 nm, or about 110 nm±40 nm. As used herein, the mean diameter of the liposomes in the composition is determined by dynamic light scattering (DLS).

All integer size ranges and integer subranges within the size range of 50 nm to 200 nm are specifically contemplated herein as exemplary mean diameters of liposomes in compositions of the invention. For purposes of evaluating particle size, size measurements by Dynamic Light Scattering at 900 is used.

In some embodiments the liposomes in the composition have a mean diameter falling within a size range, with the lower end of the size range being any size selected from about 50 nm, 51 nm, 52 nm, 53 nm, 54 nm, 55 nm, 5 nm, 57 nm, 58 nm, 59 nm, 60 nm, 61 nm, 62 nm, 63 nm, 64 nm, 65 nm, 6 nm, 67 nm, 68 nm, 69 nm, 70 nm, 71 nm, 72 nm, 73 nm, 74 nm, 75 nm, 7 nm, 77 nm, 78 nm, 79 nm, 80 nm, 81 nm, 82 nm, 83 nm, 84 nm, 85 nm, 8 nm, 87 nm, 88 nm, 89 nm, 90 nm; and with the upper end of the size range being any size sleeted from about 200 nm, 190 nm, 175 nm, 170 nm, 165 nm, 160 nm, 155 nm, 150 nm, 145 nm, 144 nm, 143 nm, 142 nm, 141 nm, 140 nm, 139 nm, 138 nm, 137 nm, 13 nm, 135 nm, 134 nm, 133 nm, 132 nm, 131 nm, 130 nm, 129 nm, 128 nm, 127 nm, 12 nm, 125 nm, 124 nm, 123 nm, 122 nm, 121 nm, 120 nm, 119 nm, 118 nm, 117 nm, 11 nm, 115 nm, 114 nm, 113 nm, 112 nm, 111 nm, 110 nm, 109 nm, 108 nm, 107 nm, 10 nm, 105 nm, 104 nm, 103 nm, 102 nm, 101 nm, 100 nm, 99 nm, 98 nm, 97 nm, 9 nm, 95 nm, 94 nm, 93 nm, 92 nm, 91 nm, or 90 nm.

In embodiments, the mean diameter of the liposomes in the composition is within a range of about 50 nm to about 200 nm. In embodiments, the mean diameter of the liposomes in the composition is within a range of about 50 nm to about 170 nm, or about 70 nm to about 150 nm, or about 90 to about 130 nm, or about 110 nm±60 nm, or about 110 nm±40 nm, or about 110 nm±20 nm.

In some embodiments, the composition exhibits a measure of uniformity in size/mass of the liposomes in the formulation, which can be expressed by measurement (or derivation) of polydispersity index (PdI). PdI is defined as the square of the standard deviation of the particle diameter distribution divided by the mean particle diameter, as set forth in the following formula, in which “a” is the radius of the particle:

$$PdI = {( \frac{σ}{2 a} )}^{2} .$$

PdI is used to estimate the uniformity of a particle distribution, and larger PdI values corresponds to a larger size distribution in the particles of the sample. For example, in some embodiments, the composition has a PdI less than or equal to about 0.4, or less than or equal to about 0.35, or less than or equal to about 0.3, or less than or equal to about 0.25, or less than or equal to about 0.23, or less than or equal to about 0.22, or less than or equal to about 0.21, or less than or equal to about 0.20, or less than or equal to about 0.19, or less than or equal to about 0.18, or less than or equal to about 0.17, or less than or equal to about 0.16, or less than or equal to about 0.15, or less than or equal to about 0.14 or less than or equal to about 0.13 or less than or equal to about 0.12, or less than or equal to about 0.11 or less than or equal to about 0.10. In some variations, the PdI is in the range of 0.01-0.40; or in the range of 0.02-0.35; or in the range of 0.03-0.30; or in the range of 0.04-0.25; or in the range of 0.05-0.20; or in the range of 0.0-0.15.

For purposes of calculating PdI herein, particle diameter (or radius) obtained by particle size measurements by Dynamic Light Scattering at 900 is used.

Compound A can be present in the liposome in any amount that provides one or more of: a liposome having a similar efficacy as a corresponding soluble, Compound A that is not incorporated within a liposome, or provides a superior efficacy; or provides a therapeutic window (between minimum effective concentration and toxic concentration). In some embodiments, Compound A is present in the liposome in an amount of about 0.1 wt. % to about 20 wt. %, based on the total weight of the liposome. In some embodiments, Compound A is present in an amount of about 0.5 wt. % to about 20 wt. %, or about 3 wt. % to about 8 wt. %, or about 4 wt. % to about 13 wt. %, based on the total weight of the liposome. For example, Compound A can be present in an amount of about 1 wt. %, about 2 wt. %, about 3 wt. %, about 4 wt. %, about 5 wt. %, about wt. %, about 7 wt. %, about 8 wt. %, about 9 wt. %, about 10 wt. %, about 11 wt. %, about 12 wt. %, about 13 wt. %, about 14 wt. %, about 15 wt. %, about 1 wt. %, about 17 wt. %, about 18 wt. %, about 19 wt. %, about 20 wt, based on the total weight of the liposome. In embodiments, Compound A is present in an amount of about 0.5 wt. % to about 15 wt. %, or about 1 wt. % to about 12 wt. %, or about 5 wt. % to about 12 wt. %, based on the total weight of the liposome. A weight percentage of Compound A is chosen to produce an effective immunomodulatory response with minimal toxic side effects. If too little Compound A is present relative to total liposome, the liposome will not stimulate a sufficiently robust response. If too much of Compound A is present relative to total liposome, the excess Compound A will compete for binding sites and may cause the liposomes to block each other from binding. Further, too much Compound A may result in toxicity issues, e.g., wherein Compound A is present in more than 20 wt. %, based on the total weight of the liposome.

In the context of evaluating weight percent of liposomal components, the weight of the liposomal membrane is considered, i.e., the ingredients used to make the liposomal membrane, without consideration of the weight of the aqueous or hydrophilic lumen of the liposome.

In some embodiments, the liposomal composition is provided in a concentrated formulation. For example, a formulation is contemplated that includes Compound A in a concentration of 0.01 to 50 mg/ml. Within this range, all subranges are contemplated. For example, subranges with exemplary lower end concentrations of 0.01 mg/ml, 0.025 mg/ml, 0.05 mg/ml, 0.075 mg/ml, 0.1 mg/ml, 0.125 mg/ml, 0.15 mg/ml, 0.2 mg/ml, 0.25 mg/ml, 0.3 mg/ml, 0.4 mg/ml, 0.5 mg/ml, 0.75 mg/ml, and 1.0 mg/ml are contemplated; and subranges with exemplary upper end concentrations (that are higher than the selected lower end concentration) of 0.5 mg/ml, 0.75 mg/ml, 0.0 mg/ml, 1.25 mg/ml, 1.5 mg/ml, 2 mg/ml, 2.5 mg/ml, 5.0 mg/ml, 10 mg/ml, 5 mg/ml, 20 mg/ml, 25 mg/ml, 30 mg/ml, and 40 mg/ml are contemplated. Concentrated formulations are contemplated for manufacture, storage, and distribution.

In some embodiments, a lower concentration liposomal composition is contemplated. For example, in some embodiments, the lower concentration composition is formulated by dilution of a concentrated formulation with a pharmaceutically acceptable diluent, such as an isotonic saline solution, for purposes of administration. In some embodiments the composition includes Compound A in a concentration of about 0.001 ng/mL to about 10,000 ng/mL. For example, compositions are contemplated that include Compound A in a concentration of about 0.01 ng/mL to about 5000 ng/mL, or about 0.1 ng/mL to about 5000 ng/mL, or about 1 ng/mL to about 1000 ng/mL, or about 10 ng/mL to about 100 ng/mL. Exemplary concentrations of Compound A that are contemplated include 0.01 ng/mL, 0.5 ng/mL, 0.1 ng/mL, 0.25 ng/mL, 0.5 ng/mL, 0.75 ng/mL, 0.1 ng/mL, 0.25 ng/mL, 0.5 ng/mL, 0.75 ng/mL, 1 ng/mL, 2.5 ng/mL, 5.0 ng/mL, 7.5 ng/mL, 10 ng/mL, 25 ng/mL, 50 ng/mL, 75 ng/mL, 100 ng/mL, 250 ng/mL, 500 ng/mL, 750 ng/mL, 1 μg/ml, 2 μg/ml, 2.5 μg/ml, 5 μg/ml, 7.5 μg/ml, 10 μg/ml, 25 μg/ml, 50 μg/ml, 75 μg/ml, and 100 μg/ml. Data in pre-clinical mouse studies has shown that a concentration as low as 0.01 ng/mL is efficacious in a mouse with 100 uL IV injection in the tail.

Compound A described herein can be synthesized using any means known to the synthetic organic chemist, such as the synthetic methods described in U.S. Pat. No. 9,700,532, incorporated herein by reference.

In some embodiments, the two or more lipids as disclosed herein can include a lipid including a saturated distearoyl moiety and a lipid including a phosphoglycerol moiety.

For example, embodiments are contemplated wherein at least one lipid is selected from phosphatidylcholine (PC), phosphatidic acid (PA), phosphatidylethanolamine (PE), phosphatidylglycerol (PG), phosphatidylserine (PS), phosphatidylinositol (PI), dimyristoyl phosphatidyl choline (DMPC), distearoylphosphatidyl choline (DSPC), dipalmitoyl phosphatidyl choline (DPPC), dimyristoyl phosphatidyl glycerol (DMPG), distearoylphosphatidyl glycerol (DSPG), dioleoyl phosphatidyl glycerol (DOPG), dipalmitoylphosphatidylglycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoylphosphatidyl serine (POPS), palmitoyloleoyl-phosphatidylethanolamine (POPE), dioleoyl-phosphatidylethanolamine 4-(N-maleimidomethyl-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoylphosphatidylcholine (DSPC), dioleoylphosphatidylcholine (DOPC), dipalmitoylphosphatidylcholine (DPPC), and combinations thereof.

In embodiments, the lipid including the saturate distearoyl moiety can further include a phophocholine moeity, a phosphoglycerol moiety, a phosphate moiety, a phosphotidylethanolamine, a phosphotidyl acetamide moiety, a polyethylene glycol moiety, or the like.

In some embodiments a sterol is contemplated, such as cholesterol, ergosterol, lanosterol, and combinations thereof.

In embodiments, the lipid including the phosphoglycerol moiety can further include a saturated distearoyl moiety, a dimyristoyl moiety, or palmitoyl moiety. For example, the two or more lipids disclosed herein can include 1,2-distearoyl-sn-glycero-3-phosphocholine (DSPC) and 1,2-distearoyl-sn-glycero-3-phospho-rac-glycerol (DSPG).

In some embodiments, the two or more lipids as disclosed herein can include any of the following lipid pairs:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); or
(f) 1,2-dimyristoyl-sn-glycero-3-phosphoglycerol (DMPG) and cholest-5-en-30-ol (CHOL).

In some embodiments, the two or more lipids as disclosed herein can include any of the following lipid pairs:
(g) 1,2-distearoyl-sn-glycero-3-phosphocholine (DSPC) and 1,2-dipalmitoyl-sn-glycero-3-phosphocholine (DPPC);
(h) 1,2-distearoyl-sn-glycero-3-phosphocholine (DSPC) and 1,2-dipalmitoyl-sn-glycero-[phosphor-rac-(1-glycerol)](DPPG); or
(i) 1,2-dipalmitoyl-sn-glycero-3-phosphocholine (DPPC) and 1,2-dipalmitoyl-sn-glycero-[phosphor-rac-(1-glycerol)](DPPG).

In some embodiments, the two or more lipids comprise 1-palmitoyl-2-oleoyl-sn-glycero-3-phosphocholine (POPC) and dimethyldioctadecylammonium bromide (DDAB). In some embodiments, the liposome comprises two lipids including 1,2-distearoyl-sn-glycero-3-phosphocholine (DSPC) and 1,2-distearoyl-sn-glycero-3-phospho-rac-glycerol (DSPG). In embodiments, the two or more lipids can include (a) 1,2-distearoyl-sn-glycero-3-phosphocholine (DSPC) and 1,2-distearoyl-sn-glycero-3-phospho-rac-glycerol (DSPG); (b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol); or (c) 1-palmitoyl-2-oleoyl-sn-glycero-3-phosphocholine (POPC) and dimethyldioctadecylammonium bromide (DDAB). In embodiments, the two or more lipids include 1,2-distearoyl-sn-glycero-3-phosphocholine (DSPC) and 1,2-distearoyl-sn-glycero-3-phospho-rac-glycerol (DSPG). In embodiments, the two or more lipids include 1,2-distearoyl-sn-glycero-3-phosphocholine (DSPC), 1,2-distearoyl-sn-glycero-3-phospho-rac-glycerol (DSPG), and cholest-5-en-30-ol (CHOL).

In some embodiments, the liposomes are substantially devoid of cholesterol, plant sterols, and polyethylene glycolated lipids (PEG lipids). As used herein, “substantially devoid” refers to less than 10 wt % In some embodiments, the liposomes are completely devoid of cholesterol, plant sterols, and polyethylene glycolated lipids (PEG lipids).

In embodiments, the two or more lipids comprised in the liposome can further comprise any lipid that is capable of forming a liposome with Compound A described herein, as long as the resulting liposome has at least a similar or increased efficacy as a corresponding soluble Compound A that is not incorporated within a liposome. For example, one or more lipids as described in U.S. Patent Application Publication No. 2014/0050780A1, which is incorporated herein by reference, can be included in the liposome. In embodiments, the liposome can include three lipids. In embodiments, the liposome can include four lipids. The two or more lipids each can independently have a net positive charge (i.e., a cationic lipid), a net negative charge (i.e., an anionic lipid), no charges (i.e., a nonionic lipid), or an equal number of positive and negative charges (i.e., a zwitterionic lipid). The lipids can include those lipids that form a bilayer themselves or with Compound A, and those lipids that do not form a bilayer alone, but they can be included as part of a stable bilayer made from one or more other lipids.

In embodiments, any lipid described herein is additionally contemplated to be a salt thereof, for example, an ammonium salt, a sodium salt, a sodium/ammonium salt, a lithium salt, a potassium salt, or the like.

The amount of lipid that can be present in the liposome is about 75 wt. % to about 99.9 wt. %, or about 80 wt. % to about 98 wt. %, or about 80 wt. % to about 95 wt. %, or about 85 wt. % to about 95 wt. %, based on the total weight of the liposome. In some embodiments, the lipid is present in an amount of about 75 wt. % to about 99 wt. %, or about 75 wt. % to about 98 wt. %, or about 75 wt. % to about 95 wt. %, or about 87 wt. % to about 93 wt. %, based on the total weight of the liposome. For example, the lipid can be present in an amount of about 75 wt. %, about 7 wt. %, about 77 wt. %, about 78 wt. %, about 79 wt. %, about 80 wt. %, about 81 wt. %, about 82 wt. %, about 83 wt. %, about 84 wt. %, about 85 wt. %, about 8 wt. %, about 87 wt. %, about 88 wt. %, about 89 wt. %, about 90 wt. %, about 91 wt. %, about 92 wt. %, about 93 wt. %, about 94 wt. %, about 95 wt. %, about 9 wt. %, about 97 wt. %, about 98 wt. %, about 99 wt. %, about 99.5 wt. %, or about 99.9 wt. %, based on the total weight of the liposome.

The mass ratio of Compound A to the two or more lipids can be about 1:5 to about 1:20, or about 1:7 to about 1:15, or about 1:7 to about 1:12. In some embodiments, the mass ratio of Compound A to the two or more lipids can be about 1:8 to about 1:10, or about 1:9. For example, the mass ratio of Compound A to the two or more lipids can be about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20.

In the embodiments wherein the two or more lipids includes 1,2-distearoyl-sn-glycero-3-phosphocholine (DSPC) and 1,2-distearoyl-sn-glycero-3-phospho-rac-glycerol (DSPG), the DSPC is present in an amount of about 40 wt. % to 80 wt. %, or about 40 wt. % to about 60 wt. %, or about 45 wt. % to about 55 wt. %, based on the total weight of the liposome. For example, the DSPC is present in an amount of about 40 wt. %, about 45 wt. %, about 50 wt. %, about 55 wt. %, about 60 wt. %, about 65 wt. %, about 70 wt. %, about 75 wt %, or about 80 wt. %, based on the total weight of the liposome.

In the embodiments wherein the two or more lipids comprise 1,2-distearoyl-sn-glycero-3-phosphocholine (DSPC) and 1,2-distearoyl-sn-glycero-3-phospho-rac-glycerol (DSPG), the DSPG is present in an amount of about 20 wt. % to 60 wt. %, or about 30 wt. % to about 50 wt. %, or about 35 wt. % to about 45 wt. %, based on the total weight of the liposome. For example, the DSPG is present in an amount of about 20 wt. %, about 25 wt. %, about 30 wt. %, about 35 wt. %, about 40 wt. %, about 45 wt. %, about 50 wt. %, about 55 wt. %, or about 60 wt. %, based on the total weight of the liposome.

In some embodiments, the two or more lipids further includes CHOL. In embodiments, the two or more lipids includes DSPC, DSPG, and CHOL, wherein CHOL is present in an amount of about 1 wt. % to about 20 wt. %. For example, the CHOL can be present in an amount of about 5 wt. % to about 15 wt. %, or about 8 wt. % to about 12 wt. %, such as about 10 wt. %. In embodiments, the two or more lipids include DSPC, DSPG, and CHOL, wherein DSPC is present in an amount of about 40 wt % to about 50 wt %, the DSPG is present in an amount of about 30 wt % to about 40 wt %, and the CHOL is present in an amount of about 5 wt % to about 15 wt %, such as DSPC in an amount of about 45 wt %, DSPG in an amount of about 35 wt %, and CHOL in an amount of about 10 wt %.

In embodiments, the two or more lipids include 1-palmitoyl-2-oleoyl-sn-glycero-3-phosphocholine (POPC) and 3β-[N-(N′, N′-dimethylaminoethane)-carbamoyl]cholesterol hydrochloride (DC-Chol). In embodiments, the POPC is present in an amount of about 40 wt. % to 80 wt. %, or about 45 wt. % to about 65 wt. %, or about 50 wt. % to about 60 wt. %, based on the total weight of the liposome. In embodiments, the DC-Chol is present in an amount of about 15 wt. % to 55 wt. %, or about 20 wt. % to about 50 wt. %, or about 25 wt. % to about 35 wt. %, based on the total weight of the liposome.

In embodiments, the two or more lipids include 1-palmitoyl-2-oleoyl-sn-glycero-3-phosphocholine (POPC) and dimethyldioctadecylammonium bromide (DDAB). In embodiments, the POPC is present in an amount of about 40 wt. % to 80 wt. %, or about 45 wt. % to about 65 wt. %, or about 50 wt. % to about 60 wt. %, based on the total weight of the liposome. In embodiments, the DC-Chol is present in an amount of about 15 wt. % to 55 wt. %, or about 20 wt. % to about 50 wt. %, or about 30 wt. % to about 40 wt. %, based on the total weight of the liposome.

In embodiments, the two or more lipids include L-α-phosphatidylglycerol (EPG) and 1,2-dioleoyl-sn-glycero-3-ethylphosphocholine (EPC). In embodiments, the EPG is present in an amount of about 40 wt. % to 80 wt. %, or about 55 wt. % to about 75 wt. %, or about 60 wt. % to about 70 wt. %, based on the total weight of the liposome. In embodiments, the EPC is present in an amount of about 10 wt. % to 50 wt. %, or about 15 wt. % to about 40 wt. %, or about 15 wt. % to about 30 wt. %, based on the total weight of the liposome.

In embodiments, the two or more lipids comprise 1-palmitoyl-2-oleoyl-sn-glycero-3-phosphocholine (POPC) and 1,2-dioleoyl-3-trimethylammonium-propane (DOTAP). In embodiments, the POPC is present in an amount of about 40 wt. % to 80 wt. %, or about 40 wt. % to about 65 wt. %, or about 45 wt. % to about 55 wt. %, based on the total weight of the liposome. In embodiments, the DOTAP is present in an amount of about 20 wt. % to 50 wt. %, or about 25 wt. % to about 45 wt. %, or about 35 wt. % to about 45 wt. %, based on the total weight of the liposome.

In embodiments, the two or more lipids include 1,2-dimyristoyl-sn-glycero-3-phosphoglycerol (DMPG) and cholest-5-en-30-ol (CHOL). In embodiments, the DMPG is present in an amount of about 40 wt. % to 80 wt. %, or about 50 wt. % to about 75 wt. %, or about 60 wt. % to about 70 wt. %, based on the total weight of the liposome. In embodiments, the CHOL is present in an amount of about 10 wt. % to 50 wt. %, or about 15 wt. % to about 35 wt. %, or about 15 wt. % to about 30 wt. %, based on the total weight of the liposome.

In embodiments, the liposome further comprises a liposomal buffer. For example, in embodiments, the liposomal buffer can include phosphate, sodium chloride, sucrose, or a combination thereof. In embodiments, the liposomal buffer includes sodium phosphate and sodium chloride. In embodiments, the liposomal buffer includes sodium chloride and sucrose. In embodiments, the liposomal buffer includes sodium chloride, phosphate and sucrose. In embodiments, the liposomal buffer is pH of about to 7, or about 6.3 to about 6.7, such as 6.5.

### Antigen-Carrying Liposomes

In some embodiments, the liposomes described herein comprise at least one antigen, such as, for example, a viral antigen, a bacterial antigen, a fungal antigen, a tumor antigen, or mixtures thereof. The exact amount of the antigen with respect to the liposome depends on the composition and purpose of the antigen, and can be determined by one skilled in the art. In these embodiments, the liposomes described herein can be used as immunostimulants or adjuvants to produce a protective immune response, such as a B-cell response, an IgG antibody response, a T-cell response, or a CTL response to the administered antigen.

The antigen can be any antigenic material that is suitable for treatment of a particular disease. In some embodiments, the liposome described herein comprises an antigen and is used to treat cancer. The antigen can be a full-length protein antigen, a long peptide antigen (i.e., a peptide that comprises at least 25 amino acids, such as 27-75, 25-50, 25-40, or 25-30 amino acids), or a short peptide antigen (i.e., a peptide that comprises 6-25 amino acids, such as 6-25, 8-25, 10-25, or 15-20 amino acids). In these embodiments, the antigen can be a tumor associated peptide or protein that induces or enhances immune response and is derived from tumor associated genes and encoded proteins including, for example, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (AGE-B4), tyrosinase, brain glycogen phosphorylase, Melan-A, MAGE-C1, MAGE-C2, NY-ESO-1, LAGE-1, SSX-1, SSX-2(HOM-MEL-40), SSX-1, SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomeras, GnTV, Herv-K-mel, Lage-1, Mage-C2, NA-88, /Lage-2, SP17, and TRP2-Int2, (MART-I), gp100 (Pmel 17), TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens Eand E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, .beta.-Catenin, CDK4, Mum-1, p16, TAGE, PSMA, PSCA, CT7, telomerase, 43-9F, 5T4, 791Tgp72, .alpha.-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS. For example, antigenic peptides characteristic of tumors include those listed in International Patent Application Publication No. WO 20000/020581 and U.S. Patent Application Publication No. 2010/0284965, which are each incorporated herein by reference. In some embodiments, the antigen is a tumor antigen selected from the group consisting of 1UCL M AG, B AGE, RAGE, CAGE, SSX-2, NY-ESO-1, PRAME, PSMA, tyrosinase, melan-A, and mixtures thereof. In some embodiments, the tumor antigen is selected from the group consisting of P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE and mixtures thereof. In some embodiments, the tumor antigen includes NY-ESO-1 or an antigenic fragment thereof. In some variations, the tumor antigen is a mammalian protein. In some variations, the tumor antigen is a human protein. In some variations, the full-length protein is employed as the antigen. In some variations, peptides comprising an antigenic fragment of these proteins is used as the tumor antigen. In some variations, antigenic fragments of these antigens are used as the tumor antigen.

Small, hydrophilic antigens can be encapsulated within the core of the liposome. Small or large hydrophobic antigens can be noncovalently associated (e.g., through hydrophobic interactions) with the nonpolar portion of the lipid bilayer, small or large charged antigens can be attached (e.g., through electrostatic interactions) to a charged portion on the outside of the lipid bilayer, and small or large hydrophobic or hydrophilic antigens also can be covalently linked to any portion of the liposomal membrane.

In embodiments when the liposome described herein is carrying an antigen, the lipid used to form the liposome is dictated by the size and charge of the antigen (e.g., small, hydrophilic, hydrophobic, positively charged, or negatively charged antigens).

In embodiments when a positively-charged antigen is noncovalently associated with the liposomal membrane, at least one lipid of the two or more lipids preferably has a net negative charge and is selected from the group consisting of an anionic sphingosine (e.g., a dimethyl sphingosine-1-phosphate, a ceramide phosphate, a dihydroceramide phosphate, a ganglioside, and a sulfatide), an anionic phospholipid (e.g., a phosphatidic acid, a phosphatidylglycerol, a phosphatidylinositol, an inositol phosphate, a cardiolipin, a bis(monoacylglycero)phosphate, an anionic detergent that is not a sphingolipid or a phospholipid, and an anionic bioactive lipid, such as, for example, an adjuvant, a liponucleotide, a TLR-4 agonist, and a diacylglycerol pyrophosphate.

In embodiments when a negatively charged antigen is noncovalently associated with the liposomal membrane, at least one lipid of the two or more lipids preferably has a net positive charge. In these embodiments, the cation lipid can be cationic sphingosine (e.g., a trimethyl sphingosine, a trimethyl phytosphingosine, and a pyridinium ceramide), a cationic lipid that is not a sphingosine (e.g., 3β-[N-(N′,N′-dimethylaminoethane)-carbamoyl]cholesterol hydrochloride (DC-Cholesterol-HCl), 1,2-dioleoyl-3-trimethylammonium-propane (chloride salt) (18:1 TAP, DOTAP), 1,2-dioleoyl-3-trimethylammonium-propane (methyl sulfate salt) (18:1 TAP, DOTAP, MS Salt), 1,2-dimyristoyl-3-trimethylammonium-propane (chloride salt) (14:0 TAP), 1,2-dipalmitoyl-3-trimethylammonium-propane (chloride salt) (16:0 TAP), 1,2-stearoyl-3-trimethylammonium-propane (chloride salt) (18:0 TAP), Transfection Reagent I (i.e., contains DOTAP:DOPE in 1:1 w/w ratio), 1,2-dioleoyl-3-dimethylammonium-propane (DODAP) (18:1 DAP), 1,2-dimyristoyl-3-dimethylammonium-propane (14:0 DAP), 1,2-dipalmitoyl-3-dimethylammonium-propane (16:0 DAP), 1,2-distearoyl-3-dimethylammonium-propane (18:0 DAP), dimethyldioctadecylammonium (Bromide Salt) (18:0 DDAB), 1,2-dilauroyl-sn-glycero-3-ethylphosphocholine (chloride salt) (12:0 EPC, C1 Salt), 1,2-dimyristoyl-sn-glycero-3-ethylphosphocholine (chloride salt) (14:0 EPC, C1 Salt), 1,2-dimyristoleoyl-sn-glycero-3-ethylphosphocholine (Tf salt) (14:1 EPC, Tf Salt), 1,2-dipalmitoyl-sn-glycero-3-ethylphosphocholine (chloride salt) (16:0 EPC, C1 Salt), 1,2-distearoyl-sn-glycero-3-ethylphosphocholine (chloride salt) (18:0 EPC, C1 Salt), 1,2-dioleoyl-sn-glycero-3-ethylphosphocholine (chloride salt) (18:1 EPC, C1 Salt), and 1-palmitoyl-2-oleoyl-sn-glycero-3-ethylphosphocholine (chloride salt) (16:0-18:1 EPC, C1 Salt), and a cationic bioactive lipid (e.g., dimethyldioctadecylammonium (Bromide Salt) (18:0 DDAB), a lysyl phosphatidylglycerol).

Lipids that can be used to produce a liposome having a bilayer that can non-covalently associate with a hydrophobic antigen include, but are not limited to, phosphatodylglycerol (PG), phosphatidylinositol (PI), phosphatidylserine (PS), cardiolipin and phosphatidic acid (PA).

### Therapeutic Agents

In some embodiments, the liposome further comprises at least one therapeutic agent in the core of the liposome. In some embodiments, the at least one therapeutic agent is selected from the group of an immune modulator, a Toll-like receptor agonist, a Nod ligand, an anti-viral agent, an antifungal agent, an antibiotic, an antiviral antibody, a cancer immune therapeutic, a chemotherapy agent, a kinase inhibitor, a cytotoxic agent, an anti-asthmatic agent, an antihistamine agent, an anti-inflammatory agent, a vaccine adjuvant, a second liposome, an artificial antigen presenting cell, a cytokine or chemokine blocking antibody, and combinations thereof.

In some embodiments, the composition further comprises at least one therapeutic agent. In some embodiments, the at least one therapeutic agent is selected from the group of an immune modulator, a Toll-like receptor agonist, a Nod ligand, an anti-viral agent, an antifungal agent, an antibiotic, an antiviral antibody, a cancer immune therapeutic, a chemotherapy agent, a kinase inhibitor, a cytotoxic agent, an anti-asthmatic agent, an antihistamine agent, an anti-inflammatory agent, a vaccine adjuvant, a second liposome, an artificial antigen presenting cell, a cytokine or chemokine blocking antibody, and combinations thereof.

In some embodiments, the therapeutic agent comprises a hydrophobic or amphiphilic agent that intercalates or integrates into the phospholipid bilayer. In some embodiments, the at least one therapeutic agent includes a hydrophilic agent contained in the lumen of the liposome.

In some embodiments, the therapeutic agent is selected from small molecules, nucleic acids, RNA, DNA, aptamers, proteins, peptides, and a mixture thereof. In some embodiments, the therapeutic agent comprises an RNA. In some embodiments, the therapeutic agent comprises a short interfering RNA (siRNA) useful for modulating gene expression in a target cell. In some embodiments, the therapeutic agent comprises a messenger RNA (mRNA) for expression in a cell. In some embodiments, the RNA is a micro RNA (miRNA). In some embodiments, the therapeutic agent comprises an anti-cancer therapeutic. In some embodiments, the therapeutic agent comprises an immunostimulant or encodes an immunostimulant.

The therapeutic agent, when present, is present in a suitable amount. For example, the therapeutic agent(s) can be present in the liposome in any amount that provides one or more of: a liposome having a similar efficacy as the therapeutic agent(s) that is not incorporated within a liposome, or provides a superior efficacy; or provides a therapeutic window (between minimum effective concentration and toxic concentration). In some embodiments, the therapeutic agent is present in the liposome in an amount of about 0.1 wt. % to about 20 wt. %, based on the total weight of the liposome. In some embodiments, the therapeutic agent is present in an amount of about 0.5 wt. % to about 20 wt. %, or about 3 wt. % to about 8 wt. %, or about 4 wt. % to about 13 wt. %, based on the total weight of the liposome. For example, the therapeutic agent can be present in an amount of about 1 wt. %, about 2 wt. %, about 3 wt. %, about 4 wt. %, about 5 wt. %, about wt. %, about 7 wt. %, about 8 wt. %, about 9 wt. %, about 10 wt. %, about 11 wt. %, about 12 wt. %, about 13 wt. %, about 14 wt. %, about 15 wt. %, about 1 wt. %, about 17 wt. %, about 18 wt. %, about 19 wt. %, about 20 wt, based on the total weight of the liposome. In embodiments, the therapeutic agent is present in an amount of about 0.5 wt. % to about 15 wt. %, or about 1 wt. % to about 12 wt. %, or about 5 wt. % to about 12 wt. %, based on the total weight of the liposome. A weight percentage of therapeutic agent is chosen to produce an effective therapeutic response with minimal toxic side effects. If too little therapeutic agent is present relative to total liposome, the liposome will not stimulate a sufficiently robust response. If too much of the therapeutic agent is present relative to total liposome, the excess therapeutic agent may compete for binding sites and may cause the liposomes to block each other from binding. Further, too much therapeutic agent may result in toxicity issues, e.g., wherein the therapeutic agent is present in more than 20 wt. %, based on the total weight of the liposome.

### Compositions and Routes of Administration

As herein, the compositions described herein include a pharmaceutically acceptable excipient, diluent, adjuvant, or carrier. The disclosed compositions comprising Compound A and/or therapeutic agent(s) is administered by any route that permits treatment of the disease or condition.

The composition comprising Compound A may be delivered to a patient using any standard route of administration, including parenterally, such as intravenously (IV), intraarterially (IA), perivascularly, intraperitoneally (IP), intrapulmonary, subcutaneously (SC) or intramuscularly (IM), intrathecally, topically, transdermally, rectally, orally, nasally or by inhalation. In some embodiments, the composition as disclosed herein is administered to a patient intravenously or intramuscularly. In embodiments, the composition is administered intravenously. Slow-release formulations may also be prepared from the agents described herein in order to achieve a controlled release of the active agent (Compound A) in contact with the body fluids in the gastrointestinal tract, and to provide a substantial constant and effective level of the active agent in the blood plasma.

In cancer patients, intratumoral injection is specifically contemplated as an embodiment.

Administration may take the form of single dose administration, or the compositions as disclosed herein can be administered over a period of time, either in divided doses or in a continuous-release formulation or administration method (e.g., a pump). However, the compositions of the embodiments administered to the subject, the amounts of the composition administered, and the route of administration chosen is selected to permit efficacious treatment of the disease condition.

In embodiments, the compositions are formulated with one or more pharmaceutically acceptable excipient, carriers, solvents, stabilizers, adjuvants, diluents, or the like, depending upon the particular mode of administration and dosage form. The compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. In embodiments, the compositions may comprise a therapeutically or prophylactically effective amount of Compound A as described herein, together with one or more pharmaceutically acceptable excipients. Optionally, the compositions may comprise a combination of the compounds described herein, or may include a second active ingredient useful in the treatment or prevention of bacterial infection (e.g., anti-bacterial or anti-microbial agents).

Compositions disclosed hereins, e.g., for parenteral or oral administration, are most typically solids, liquid solutions, suspensions, while inhalable formulations for pulmonary administration are generally liquids or powders. Composition disclosed herein can also be formulated as a lyophilized solid that is reconstituted with a physiologically compatible solvent prior to administration. Lyophilization (freeze-drying) removes water by sublimation at low temperature and pressure, and preserves sensitive materials such as drugs, vaccines and probiotic cultures. Compositions disclosed herein can be formulated as a solution that is reconstituted with a physiologically compatible solvent prior to administration for intravenous or intramuscular injection. Parenteral injection or extended infusion, e.g., over a period of 1, 2, 3, 4, 5, 6, 7, 8, 12 or 24 hours is possible.

In some embodiments, the composition described herein comprise a lipid-bilayer coated particle, in place of, or in addition to, a liposome.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, sodium phosphates, including sodium phosphate monohydrate, glycerol and/or ethanol) wetting or emulsifying agents, pH buffering substances, and the like.

In another embodiment, compositions disclosed herein may be formulated as suspensions comprising Compound A and two or more lipids of the embodiments in admixture with at least one pharmaceutically acceptable excipient suitable for the manufacture of a suspension.

The composition disclosed herein may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by PEGylation.

In some embodiments, the compounds described herein may be formulated for oral administration in a lipid-based formulation suitable for low solubility compounds.

An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella Minnesota Re 595 lipopolysaccharide; saponins including QS21 (SmithKlineBeecham), a pure QA-21 saponin purified from Quillja saponaria extract; DQS21, described in PCT application WO96/33739 (SmithKline Beecham); QS-7, QS-17, QS-18, and QS-L1 (So et al., MoI Cells (1997) 7:178-186); ISCOMATRIX adjuvant, a cage-like structure composed of saponin, phospholipid, and cholesterol (see, e.g., Maraskovsky et al., Clin. Cancer Res. (2004) 10:2879-2890); incomplete Freund's adjuvant; complete Freund's adjuvant; montanide; alum; CpG oligonucleotides (see e.g. Kreig et al., Nature 374:546-9, 1995) and other immunostimulatory oligonucleotides including poly-IC and poly-ICLC (Hiltonol®); and various water-in-oil emulsions prepared from biodegradable oils such as squalene and/or tocopherol.

### Methods of Treatment

The disclosed liposomes can be suitable for stimulating an immune response. In various embodiments, the disclosure provides methods of stimulating an immune response in a mammalian subject comprising administering to the subject the disclosed compositions.

Provided herein are methods of treating different types of cancer in a subject (e.g., a mammal) in need thereof including administering to the subject the composition as described herein in a therapeutically effective amount. In some embodiments, the subject is a mammalian subject. In some cases, the mammalian subject is a human subject. Practice of methods described herein in other mammalian subjects, especially mammals that are conventionally used as models for demonstrating therapeutic efficacy in humans (e.g., primate, porcine, canine, or rabbit animals), is also contemplated. It is contemplated that the mice that were used in pre-clinical studies may be 1000-fold or more sensitive to Compound A than humans, which provides guidance for adjusting dosing. Standard dose-response studies are used to optimize dose and dosing schedule.

Provided herein is the use of the compositions disclosed herein in the manufacture of a medicament, such as those described above, for treatment of cancer in a mammalian subject.

The disclosed methods are useful for treating cancer, for example, inhibiting cancer growth, including complete cancer remission, for inhibiting cancer metastasis, and for promoting cancer resistance. The term “cancer growth” generally refers to any one of a number of indices that suggest change within the cancer to a more developed form. Thus, indices for measuring an inhibition of cancer growth include but are not limited to a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens.

The term “cancer resistance” refers to an improved capacity of a subject to resist cancer growth, in particular growth of a cancer already had. In other words, the term “cancer resistance” refers to a decreased propensity for cancer growth in a subject.

In one aspect, the cancer comprises a solid tumor, for example, a carcinoma and a sarcoma. Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. The invention also provides methods of treatment of cancers of myeloid or lymphoid systems, including leukemias, lymphomas, and other cancers that typically are not present as a tumor mass, but are distributed in the vascular or lymphoreticular systems. Further contemplated are methods for treatment of adult and pediatric oncology, growth of solid tumors/malignancies, myxoid and round cell carcinoma, locally advanced tumors, cancer metastases, including lymphatic metastases. The cancers listed herein are not intended to be limiting. Age (child and adult), sex (male and female), primary and secondary, pre- and post-metastatic, acute and chronic, benign and malignant, anatomical location cancer embodiments and variations are contemplated targets. Cancers are grouped by embryonic origin (e.g., carcinoma, lymphomas, and sarcomas), by organ or physiological system, and by miscellaneous grouping. Particular cancers may overlap in their classification, and their listing in one group does not exclude them from another.

Carcinomas that may targeted include adrenocortical, acinar, acinic cell, acinous, adenocystic, adenoid cystic, adenoid squamous cell, cancer adenomatosum, adenosquamous, adnexel, cancer of adrenal cortex, adrenocortical, aldosterone-producing, aldosterone-secreting, alveolar, alveolar cell, ameloblastic, ampullary, anaplastic cancer of thyroid gland, apocrine, basal cell, basal cell, alveolar, comedo basal cell, cystic basal cell, morphea-like basal cell, multicentric basal cell, nodule-ulcerative basal cell, pigmented basal cell, sclerosing basal cell, superficial basal cell, basaloid, basosquamous cell, bile duct, extrahepatic bile duct, intrahepatic bile duct, bronchioalveolar, bronchiolar, bronchioloalveolar, bronchoalveolar, bronchoalveolar cell, bronchogenic, cerebriform, cholangiocellular, chorionic, choroids plexus, clear cell, cacogenic anal, colloid, comedo, corpus, cancer of corpus uteri, cortisol-producing, cribriform, cylindrical, cylindrical cell, duct, ductal, ductal cancer of the prostate, ductal cancer in situ (DCIS), eccrine, embryonal, cancer en cuirasse, endometrial, cancer of endometrium, endometroid, epidermoid, cancer ex mixed tumor, cancer ex pleomorphic adenoma, exophytic, fibrolamellar, cancer fibro′sum, follicular cancer of thyroid gland, gastric, gelatinform, gelatinous, giant cell, giant cell cancer of thyroid gland, cancer gigantocellulare, glandular, granulose cell, hepatocellular, Hurthle cell, hypernephroid, infantile embryonal, islet cell carcinoma, inflammatory cancer of the breast, cancer in situ, intraductal, intraepidermal, intraepithelial, juvenile embryonal, Kulchitsky-cell, large cell, leptomeningeal, lobular, infiltrating lobular, invasive lobular, lobular cancer in situ (LCIS), lymphoepithelial, cancer medullare, medullary, medullary cancer of thyroid gland, medullary thyroid, melanotic, meningeal, Merkel cell, metatypical cell, micropapillary, mucinous, cancer muciparum, cancer mucocellulare, mucoepidermoid, cancer mucosum, mucous, nasopharyngeal, neuroendocrine cancer of the skin, noninfiltrating, non-small cell, non-small cell lung cancer (NSCLC), oat cell, cancer ossificans, osteoid, Paget's, papillary, papillary cancer of thyroid gland, periampullary, preinvasive, prickle cell, primary intraosseous, renal cell, scar, schistosomal bladder, Schneiderian, scirrhous, sebaceous, signet-ring cell, cancer simplex, small cell, small cell lung cancer (SCLC), spindle cell, cancer spongiosum, squamous, squamous cell, terminal duct, anaplastic thyroid, follicular thyroid, medullary thyroid, papillary thyroid, trabecular cancer of the skin, transitional cell, tubular, undifferentiated cancer of thyroid gland, uterine corpus, verrucous, villous, cancer villosum, yolk sac, squamous cell particularly of the head and neck, esophageal squamous cell, and oral cancers and carcinomas.

Sarcomas that may be targeted include adipose, alveolar soft part, ameloblastic, avian, botryoid, sarcoma botryoides, chicken, chloromatous, chondroblastic, clear cell sarcoma of kidney, embryonal, endometrial stromal, epithelioid, Ewing's, fascial, fibroblastic, fowl, giant cell, granulocytic, hemangioendothelial, Hodgkin's, idiopathic multiple pigmented hemorrhagic, immunoblastic sarcoma of B cells, immunoblastic sarcoma of T cells, Jensen's, Kaposi's, kupffer cell, leukocytic, lymphatic, melanotic, mixed cell, multiple, lymphangio, idiopathic hemorrhagic, multipotential primary sarcoma of bone, osteoblastic, osteogenic, parosteal, polymorphous, pseudo-kaposi, reticulum cell, reticulum cell sarcoma of the brain, rhabdomyosarcoma, rous, soft tissue, spindle cell, synovial, telangiectatic, sarcoma (osteosarcoma)/malignant fibrous histiocytoma of bone, and soft tissue sarcomas.

Lymphomas that may be targeted include AIDS-related, non-Hodgkin's, Hodgkin's, T-cell, T-cell leukemia/lymphoma, African, B-cell, B-cell monocytoid, bovine malignant, Burkitt's, centrocytic, lymphoma cutis, diffuse, diffuse, large cell, diffuse, mixed small and large cell, diffuse, small cleaved cell, follicular, follicular center cell, follicular, mixed small cleaved and large cell, follicular, predominantly large cell, follicular, predominantly small cleaved cell, giant follicle, giant follicular, granulomatous, histiocytic, large cell, immunoblastic, large cleaved cell, large noncleaved cell, Lennert's, lymphoblastic, lymphocytic, intermediate; lymphocytic, intermediately differentiated, plasmacytoid; poorly differentiated lymphocytic, small lymphocytic, well differentiated lymphocytic, lymphoma of cattle; MALT, mantle cell, mantle zone, marginal zone, Mediterranean lymphoma mixed lymphocytic-histiocytic, nodular, plasmacytoid, pleomorphic, primary central nervous system, primary effusion, small b-cell, small cleaved cell, small concleaved cell, T-cell lymphomas; convoluted T-cell, cutaneous t-cell, small lymphocytic T-cell, undefined lymphoma, u-cell, undifferentiated, aids-related, central nervous system, cutaneous T-cell, effusion (body cavity based), thymic lymphoma, and cutaneous T cell lymphomas.

Leukemias and other blood cell malignancies that may be targeted include acute lymphoblastic, acute myeloid, acute lymphocytic, acute myelogenous leukemia, chronic myelogenous, hairy cell, erythroleukemia, lymphoblastic, myeloid, lymphocytic, myelogenous, leukemia, hairy cell, T-cell, monocytic, myeloblastic, granulocytic, gross, hand mirror-cell, basophilic, hemoblastic, histiocytic, leukopenic, lymphatic, Schilling's, stem cell, myelomonocytic, monocytic, prolymphocytic, promyelocytic, micromyeloblastic, megakaryoblastic, megakaryoctyic, rieder cell, bovine, aleukemic, mast cell, myelocytic, plasma cell, subleukemic, multiple myeloma, nonlymphocytic, chronic myelogenous leukemia, chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia and chronic myelocytic leukemias.

Brain and central nervous system (CNS) cancers and tumors that may be targeted include astrocytomas (including cerebellar and cerebral), brain stem glioma, brain tumors, malignant gliomas, ependymoma, glioblastoma, medulloblastoma, supratentorial primitive neuroectodermal tumors, visual pathway and hypothalamic gliomas, primary central nervous system lymphoma, ependymoma, brain stem glioma, visual pathway and hypothalamic glioma, extracranial germ cell tumor, medulloblastoma, myelodysplastic syndromes, oligodendroglioma, myelodysplastic/myeloproliferative diseases, myelogenous leukemia, myeloid leukemia, multiple myeloma, myeloproliferative disorders, neuroblastoma, plasma cell neoplasm/multiple myeloma, central nervous system lymphoma, intrinsic brain tumors, astrocytic brain tumors, gliomas, and metastatic tumor cell invasion in the central nervous system.

Gastrointestimal cancers that may be targeted include extrahepatic bile duct cancer, colon cancer, colon and rectum cancer, colorectal cancer, gallbladder cancer, gastric (stomach) cancer, gastrointestinal carcinoid tumor, gastronintestinal carcinoid tumors, gastrointestinal stromal tumors, bladder cancers, islet cell carcinoma (endocrine pancreas), pancreatic cancer, islet cell pancreatic cancer, prostate cancer rectal cancer, salivary gland cancer, small intestine cancer, colon cancer, and polyps associated with colorectal neoplasia.

Lung and respiratory cancers that may be targeted include bronchial adenomas/carcinoids, esophagus cancer esophageal cancer, esophageal cancer, hypopharyngeal cancer, laryngeal cancer, hypopharyngeal cancer, lung carcinoid tumor, non-small cell lung cancer, small cell lung cancer, small cell carcinoma of the lungs, mesothelioma, nasal cavity and paranasal sinus cancer, nasopharyngeal cancer, nasopharyngeal cancer, oral cancer, oral cavity and lip cancer, oropharyngeal cancer; paranasal sinus and nasal cavity cancer, and pleuropulmonary blastoma.

Urinary tract and reproductive cancers that may be targeted include cervical cancer, endometrial cancer, ovarian epithelial cancer, extragonadal germ cell tumor, extracranial germ cell tumor, extragonadal germ cell tumor, ovarian germ cell tumor, gestational trophoblastic tumor, spleen, kidney cancer, ovarian cancer, ovarian epithelial cancer, ovarian germ cell tumor, ovarian low malignant potential tumor, penile cancer, renal cell cancer (including carcinomas), renal cell cancer, renal pelvis and ureter (transitional cell cancer), transitional cell cancer of the renal pelvis and ureter, gestational trophoblastic tumor, testicular cancer, ureter and renal pelvis, transitional cell cancer, urethral cancer, endometrial uterine cancer, uterine sarcoma, vaginal cancer, vulvar cancer, ovarian carcinoma, primary peritoneal epithelial neoplasms, cervical carcinoma, uterine cancer and solid tumors in the ovarian follicle), superficial bladder tumors, invasive transitional cell carcinoma of the bladder, and muscle-invasive bladder cancer.

Skin cancers and melanomas (as well as non-melanomas) that may be targeted include cutaneous T-cell lymphoma, intraocular melanoma, tumor progression of human skin keratinocytes, basal cell carcinoma, and squamous cell cancer. Liver cancers that may be targeted include extrahepatic bile duct cancer, and hepatocellular cancers. Eye cancers that may be targeted include intraocular melanoma, retinoblastoma, and intraocular melanoma. Hormonal cancers that may be targeted include parathyroid cancer, pineal and supratentorial primitive neuroectodermal tumors, pituitary tumor, thymoma and thymic carcinoma, thymoma, thymus cancer, thyroid cancer, cancer of the adrenal cortex, and ACTH-producing tumors.

Miscellaneous other cancers that may be targeted include advanced cancers, AIDS-related, anal cancer adrenal cortical, aplastic anemia, aniline, betel, buyo cheek, cerebriform, chimney-sweeps, clay pipe, colloid, contact, cystic, dendritic, cancer a deux, duct, dye workers, encephaloid, cancer en cuirasse, endometrial, endothelial, epithelial, glandular, cancer in situ, kang, kangri, latent, medullary, melanotic, mule-spinners', non-small cell lung, occult cancer, paraffin, pitch workers', scar, schistosomal bladder, scirrhous, lymph node, small cell lung, soft, soot, spindle cell, swamp, tar, and tubular cancers.

Miscellaneous other cancers that may be targeted also include carcinoid (gastrointestinal and bronchial) Castleman's disease chronic myeloproliferative disorders, clear cell sarcoma of tendon sheaths, Ewing's family of tumors, head and neck cancer, lip and oral cavity cancer, Waldenström's macroglobulinemia, metastatic squamous neck cancer with occult primary, multiple endocrine neoplasia syndrome, multiple myeloma/plasma cell neoplasm, Wilms' tumor, mycosis fungoides, pheochromocytoma, sezary syndrome, supratentorial primitive neuroectodermal tumors, unknown primary site, peritoneal effusion, malignant pleural effusion, trophoblastic neo-plasms, and hemangiopericytoma.

In embodiments, the cancer can be selected from the group of basal cell carcinoma, breast cancer leukemia, Burkitt's lymphoma, colon cancer, esophageal cancer, bladder cancer, gastric cancer, head and neck cancer, hepatocellular cancer, Hodgkin's lymphoma, hairy cell leukemia, Wilms' tumor, thyroid cancer, thymoma and thymic carcinoma, testicular cancer, T-cell lymphoma, prostate cancer, non-small cell lung cancer, liver cancer, renal cell cancer, melanoma, and combinations thereof. In embodiments, the cancer includes melanoma. In embodiments, the cancer includes non-small cell lung cancer.

Further described herein are methods of stimulating an immune response in a mammalian subject comprising administering to the subject a composition described herein. In some embodiments, the composition is administered directly to the subject in the same manner as a vaccine. In some embodiments, the compositions described herein are useful for the induction of an immune response to a tumor antigen, one or more pathogenic organisms, or other antigen as described herein.

### Dosing

The terms “therapeutically effective amount” and “prophylactically effective amount,” as used herein, refer to an amount of a composition sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

As used herein, the term “treating” includes prophylaxis of the specific disorder or condition, or alleviation of one or more symptoms associated with a specific disorder or condition and/or preventing or eliminating one or more symptoms, and/or reducing the severity of the condition and/or slowing the progression of the condition. A fully successful treatment may result in eliminating the condition (cure), but cure is not a required benchmark for a successful treatment. “Prophylaxis” refers to a preventative therapy and its effectiveness can be shown by comparison between a population that receives the prophylaxis and a population that only receives a negative control. Effective prophylaxis results in reduced incidence and/or delayed onset and/or reduced severity, compared to the control population. In the context of an infection, treating refers to slowing the spread of the infection and/or stopping the spread of the infection and/or reducing or eliminating the amount of the infective agent in a host. In the context of a cancer, an effective treatment slows the growth of the cancer and/or reduces its harmful effect on a patient and/or increases the life span of a cancer patient and/or increases the span of quality life of the patient; or causes shrinkage of a tumor or reduction in the number of cancer cells, or results in elimination of the cancer. In the context of vaccination, treatment causes the body to produce an immune response that inhibits or prevents future infection by an infective agent or reduces the infection's severity or duration or negative effects, if infection occurs.

Dosages of the therapeutic can be administered as a dose measured in micrograms or milligrams. For purposes of this paragraph, “therapeutic” refers to the amount of Compound A in the dosage. Contemplated dosages of the disclosed therapeutics include doses in the range of about 0.1 μg to about 50000 μg (50 mg).

Within the above broad guidelines, dose subranges are specifically contemplated. For example, dose ranges are contemplated with a lower end dose of Compound A of any of about 0.5 μg, or about 1 g or about 0.5 μg, or about 1 μg, or about 5 μg, or about 10 μg, or about 20 μg, or about 30 μg, or about 40 μg, or about 50 μg, or about 60 μg, or about 70 μg, or about 80 μg, or about 90 μg, or about 100 μg, or about 110 μg, or about 120 μg, or about 130 μg, or about 140 μg, or about 150 μg, or about 160 μg, or about 170 μg, about 180 μg, or about 190 μg, or about 200 μg, or about 210 μg, or about 220 μg, or about 230 μg, or about 240 μg, or about 250 μg, or about 260 μg, or about 270 μg, or about 280 μg, or about 290 μg, or about 300 μg, or about 310 μg, or about 320 μg, or about 330 μg, or about 340 μg, or about 350 μg, or about 360 μg, or about 370 μg, or about 380 μg, or about 390 μg, or about 400 μg, or about 410 μg, or about 420 μg, or about 430 μg, or about 440 μg, or about 450 μg, or about 460 μg, or about 470 μg, or about 480 μg, or about 490 μg, or about 500 μg, or about 510 μg, or about 520 μg, or about 530 μg, or about 540 g, or about 550 μg, or about 560 μg, or about 570 μg, or about 580 μg, or about 590 μg, or about 600 μg, or about 650 μg, or about 700 μg, or about 750 μg, or about 800 μg, or about 900 μg, or 950 μg, or about 1 mg, or about 2 mg, or about 3 mg, or about 4 mg, or about 5 mg, or about mg, or about 7 mg, or about 8 mg, or about 9 mg, or about 10 mg, or about 11 mg, or about 12 mg, or about 13 mg, or about 14 mg, or about 15 mg, or about 1 mg, or about 17 mg, or about 18 mg, or about 19 mg, or about 20 mg, or about 21 mg, or about 22 mg, or about 23 mg, or about 24 mg, or about 25 mg, or about 2 mg, or about 27 mg, or about 28 mg, or about 29 mg, or about 30 mg; and with a higher end dose of Compound A (that is higher than the selected lower end) of any of about 0.5 μg, or about 1 g or about 0.5 μg, or about 1 μg, or about 5 μg, or about 10 μg, or about 20 μg, or about 30 μg, or about 40 μg, or about 50 μg, or about 60 μg, or about 70 μg, or about 80 μg, or about 90 μg, or about 100 μg, or about 110 μg, or about 120 μg, or about 130 μg, or about 140 μg, or about 150 μg, or about 160 μg, or about 170 μg, about 180 μg, or about 190 μg, or about 200 μg, or about 210 μg, or about 220 μg, or about 230 μg, or about 240 μg, or about 250 μg, or about 260 μg, or about 270 μg, or about 280 μg, or about 290 μg, or about 300 μg, or about 310 μg, or about 320 μg, or about 330 μg, or about 340 μg, or about 350 μg, or about 360 μg, or about 370 μg, or about 380 μg, or about 390 μg, or about 400 μg, or about 410 μg, or about 420 μg, or about 430 μg, or about 440 μg, or about 450 μg, or about 460 μg, or about 470 μg, or about 480 μg, or about 490 μg, or about 500 μg, or about 510 μg, or about 520 μg, or about 530 μg, or about 540 μg, or about 550 μg, or about 560 μg, or about 570 μg, or about 580 μg, or about 590 μg, or about 600 μg, or about 650 μg, or about 700 μg, or about 750 μg, or about 800 μg, or about 900 μg, or 950 μg, or about 1 mg, or about 2 mg, or about 3 mg, or about 4 mg, or about 5 mg, or about mg, or about 7 mg, or about 8 mg, or about 9 mg, or about 10 mg, or about 11 mg, or about 12 mg, or about 13 mg, or about 14 mg, or about 15 mg, or about 1 mg, or about 17 mg, or about 18 mg, or about 19 mg, or about 20 mg, or about 21 mg, or about 22 mg, or about 23 mg, or about 24 mg, or about 25 mg, or about 2 mg, or about 27 mg, or about 28 mg, or about 29 mg, or about 30 mg, or about 31 mg, or about 32 mg, or about 33 mg, or about 34 mg, or about 35 mg, or about 3 mg, or about 37 mg, or about 38 mg, or about 39 mg, or about 40 mg, or about 41 mg, or about 42 mg, or about 43 mg, or about 44 mg, or about 45 mg, or about 4 mg, or about 47 mg, or about 48 mg, or about 49 mg, or about 50 mg.

Doses and dosage ranges at the higher end of this spectrum, e.g., with a minimum dose of about 1 mg, or about 5 mg, or about 10 mg, or about 15 mg, or about 20 mg, or about 25 mg, or about 30 mg, are contemplated for oncology indications; doses and dosage ranges at the lower end of the above spectrum, e.g., with a maximum dose of about 10 mg, are contemplated for non-oncology indications.

Specific ranges of doses in μg include about 1 μg to about 20000 μg, about 2 μg to about 10000 μg, about 5 μg to about 2000 μg, about 5 μg to about 1000 μg, about 10 μg to about 1000 μg, about 20 μg to about 500 μg, about 30 μg to about 200 μg, and about 50 μg to about 100 μg. The doses can be total daily amounts given to a subject or the dose given at any single time. Thus, the dose can be administered as a single dose or in divided doses throughout the day (e.g., in two, three, four, or five doses over the course of a day).

The dose of the liposomes that is administered will vary with the exact composition of the liposome. In some embodiments, it is contemplated that the liposomes are administered to result in a daily dosage of Compound A of about 0.5 g to about 50 mg, or about 1 g to about 45 mg, or about 10 g to about 40 mg, or about 50 g to about 3 mg, or about 1 g to about 20 mg, e.g., about 0.5 μg, about 1 μg, about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 480 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1 mg, about 2 mg, about 3 mg, about 4 mg, about 5 mg, about mg, about 7 mg, about 8 mg, about 9 mg, about 10 mg, about 11 mg, about 12 mg, about 13 mg, about 14 mg, about 15 mg, about 1 mg, about 17 mg, about 18 mg, about 19 mg, about 20 mg, about 21 mg, about 22 mg, about 23 mg, about 24 mg, about 25 mg, about 2 mg, about 27 mg, about 28 mg, about 29 mg, about 30 mg, about 31 mg, about 32 mg, about 33 mg, about 34 mg, about 35 mg, about 3 mg, about 37 mg, about 38 mg, about 39 mg, or about 40 mg. In some embodiments, the liposomes are administered to result in a daily dosage of the nonglycosidic ceramide in an amount of about 1 μg to about 200 μg, or about 1 mg to about 10 mg, or about 700 g to about 2 mg, or about 800 g to about 28 mg, or about 10 g to about 40 mg, about 10 g to about 5 mg. In some embodiments, the liposomes are administered in divided doses 1 to 4 times daily or in a sustained release form.

The modifier “about”—when used in the context of numerical values, should be understood to include a level of variation at least commensurate with conventional measurement tools for the parameter in question, or ten percent, whichever is greater.

Dosages of the therapeutic can alternately be administered as a dose measured in mg/kg (mg compound per kilogram of body weight for the treated subject). For purposes of this paragraph, “therapeutic” refers to the amount of Compound A in the dosage. Contemplated mg/kg doses of the disclosed therapeutics include about 0.00001 mg/kg to about 1 mg/kg. Specific ranges of doses in mg/kg include about 0.0001 mg/kg to about 0.5 mg/kg, about 0.0005 mg/kg to about 0.2 mg/kg, about 0.001 mg/kg to about 0.1 mg/kg, C. All doses within these ranges are contemplated, including doses of 0.00001 mg/kg, 0.00005 mg/kg, 0.0001 mg/kg, 0.0005 mg/kg, 0.001 mg/kg, 0.005 mg/kg, 0.01 mg/kg, 0.05 mg/kg, 0.1 mg/kg, 0.5 mg/kg, and 1 mg/kg.

Dosages of the therapeutic can alternately be administered as a dose measured in mg/m² (mg compound per m² surface area for the treated subject). To provide a frame of reference, an average or typical body surface are for an adult female is 1.0 m² and for an adult male is 1.9 m², and these numbers can be used to convert/express any of the foregoing doses or dosage ranges into doses or dosages expressed in mg/m². Exemplary contemplated mg/m² doses of the disclosed therapeutics include about 0.5 mg/m² to about 20 mg/m²; about 1 mg/m² to about 15 mg/m²; about 2 mg/m² to about 15 mg/m²; about 3 mg/m² to about 10 mg/kg; or about 4 mg/m² to about 10 mg/m².

### Combination Therapy

The methods disclosed herein can also include the use of compositions as described herein together with one or more additional therapeutic agents for the treatment of disease conditions. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together, which can be co-formulation or separate administration at essentially the same time. Various sequences of intermittent combination therapy may also be used. In some cases, a composition disclosed herein is administered and/or formulated with a second therapeutic. In some embodiments, the composition as disclosed herein and the further therapeutic or therapy are administered concurrently. In some embodiments, the composition as disclosed herein and the further therapeutic or therapy are administered separately.

The second therapeutic can be one or more of a chemotherapeutic or an immunotherapeutic agent. In some specific cases, the second therapeutic is a cytokine, an anti-inflammatory agent, a cancer vaccine, a cancer antigen, or a polynuecleotide encoding a cancer antigen. In some cases, the second therapeutic is radiation.

Contemplated chemotherapeutics for use in combination therapies as disclosed herein include aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; and enzymes such as L-asparaginase.

Contemplated biological response modifying agents for use in combination therapies as disclosed herein include, but are not limited to, interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

Contemplated immunotherapeutic agents, such as an immune checkpoint inhibitor, for use in the combination therapies disclosed herein. The immunotherapeutic agents include, but are not limited to, a Her2/neu receptor antibody such as trastuzumab (marketed as Herceptin@), an anti-CD52 antibody such as alemtuzumab (marketed as Campath®. MabCampath® or Campath-1H), an anti-CD33 antibody such as gemtuzumab linked to calicheamicin (marketed as Mylotarg®), an anti-CD20 antibody such as rituximab (marketed as Rituxan® and MabThera®), Ibritumomab tiuxetan (marketed as Zevalin®), anti-TNFα antibodies such as infliximab (marketed as Remicade®) or adalimmumab (marketed as Humira®), a soluble TNFR2 molecule such as etanercept (marketed as Enbrel®), an antibody to the CD25 chain of the IL-2 receptor such as basiliximab (marketed as Simulect®), an anti CD40/CD40L antibody such as humanized IgG1 anti-human CD40 antibody (SGN-40), an anti-CTLA-4 blocking antibody such as iplimumab (marketed as MDX-101 or MDX-010) or tremelimumab, an anti-programmed death protein 1 (PD-1) antibody (i.e., an anti-CD279 antibody), an anti-programmed cell death ligand (PDL-1) antibody, an anti-glucocorticoid-induced TNFR-related gene (GITR) antibody, an anti-OX-40 (CD134) antibody, soluble lymphocyte-activation gene 3 (also known as LAG3 or CD223)-based immune modulator such as LAG3-Ig (IMP321), Toll-like receptor agonists such as monophosphoril lipid A (MPL®), CpG, single-stranded RNA, nucleotides, nucleotide analogue, CL087 (a TLR7-specific ligand), loxoribine, polyinosine-polycytidylic acid, flagellin, resiquimod, imiquimod, gardiquimod, NOD ligands such as muramyl dipeptide, murabutide, peptidoglycan and muramyldipeptide.

In embodiments, the methods or use disclosed herein further comprise administering to the subject or using at least one checkpoint inhibitor. In embodiments, the immune checkpoint inhibitor includes PD-1 inhibitor, a PD-L1 inhibitor, or a CTLA-4 inhibitor. In some embodiments, the immune checkpoint inhibitor can include at least one PD-1 inhibitor. In embodiments, the at least one PD-1 inhibitor can be selected from Pembrolizumab, Nivolumab, Cemiplimab, and Spartalizumab. In embodiments, the at least one PD-L1 inhibitor can be selected from Atezolizumab, Avelumab, and Durvalumab. In embodiments, the at least one CTLA-4 inhibitor can be selected from Ipilimumab and Tremelimumab.

In some embodiments, the immune checkpoint inhibitor is an agent that targets the Programmed Cell Death Protein 1 (PD-1) pathway. Programmed cell death protein 1 (PD-1), also known as cluster of differentiation 279 (CD279), is a cell surface co-inhibitory receptor expressed on activated T cells, B cells and macrophages, and is a component of immune checkpoint blockade (Shinohara et al., Genomics., 23(3):704, (1994); Francisco et al., Immunol Rev., 236: 219, (2010)). PD-1 limits the activity of T cells upon interaction with its two ligands PD-L1 (also known as B7-H1; CD274) and PD-L2 (B7-DC; CD273) (Postow et al., J Clin Oncol., 33: 9, (2015)). Interaction of PD-1 with PD-L1 and PD-L2, reduces T-cell proliferation, cytokine production, and cytotoxic activity (Freeman G J et al., J Exp Med., 192:1027-34, (2000); Brown J A et al., J Immunol., 170:1257-66, (2003)).

Agents that Target the PD-1/PD-L1 Pathway

Various agents that inhibit the pathway PD-1/PD-L1 described in the art and may be used in the methods disclosed herein. Inhibitors of PD-1/PD-L1 include antibodies directed to PD-1, PD-L1 and PD-L2, PD-1/PD-L1 peptides, PD-1/PD-L1 small molecule chemical compounds, for example. (Jiang et al., Front Immunol. 2020, 12; 11:339; Guo et al. Front. Immunol., 2020; 11:1508).

### PD-1 Antibodies

Antibodies to PD-1 have been described in, for example, U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; 8,217,149; 8,952,136; 9,102,727; 10,428,146; and U.S. Pat. No. 10,752,687 (incorporated by reference in their entirety).

It is contemplated that any known antibody can be used in the present methods. In some embodiments, a murine monoclonal anti-target antibody for human PD-1 is used in the present methods. For example, InVivo MAb anti h PD-1 (BioXCell, Clone: RMP1-14, Cat. no.: BE0146). Anti-PD-1 antibodies have been shown to be effective in human therapy, see e.g., pembrolizumab (KEYTRUDA®, Merck Sharp & Dohme Corp.), nivolumab (Opdivo®, Bristol-Myers Squibb) and cemiplimab (Libtayo®, Regeneron Pharmaceuticals) which are anti-PD-1 antibodies approved for use in human therapy. These antibodies bind to the PD-1 receptor and block its interaction with its ligands, PD-L1 and PD-L2. Exemplary FDA-approved anti-PD-1/L1 antibodies are listed in Table PD-A and anti-PD-1/L1 antibodies under development are listed in Table PD-B, along with patents describing them.

Additional PD-1 antibodies in clinical development are contemplated. For example, pidilizumab (CT-011) (CureTech Ltd.); SSI-361 (Lyvgen) (Wang et al., US Patent Application Publication No. 20180346569A1); Prolgolimab (BCD-100) (Tjulandin et al., Ann Oncol. 2019; 30(suppl_11):xi33-xi47); Spartalizumab (PDR001) (Norvatis) (Naing et al., J Immunother Cancer. 2020; 8(1):e000530); GLS-010 (Song et al., Blood (2020) 13 (Supplement 1): 17); camrelizumab (SHR1210) (Jiangsu HengRui Medicine Co., Ltd.) (Markham et al., 2019 Drugs. 79 (12): 1355-1361); sintilimab (IBI308) (Innovent and Eli Lilly) (Xu et al., Journal of Clinical Oncology 36(15_suppl):ei5125-ei5125); tislelizumab (BGB-A317) (“Library Association-Annual Meeting”. The Library. si-1 (1): 215. 1889-01-01); toripalimab (JS 001) (Wang et al., Journal of Clinical Oncology 2019 37:15_suppl, 6017-6017); dostarlimab (TSR-042, WBP-285) (GlaxoSmithKline) (ClinicalTrials Identifier: NCT03981796); INCMGA00012 (MGA012) (Press release: Incyte and MacroGenics, MacroGenics Announces Development Milestone Achieved in retifanlimab (MGA012) Collaboration with Incyte. Source: MacroGenics, Inc.); AMP-224 (AstraZeneca/MedImmune and GlaxoSmithKline) (Floudas et al., Clin Colorectal Cancer. 2019); and MEDI-0680 (AMP-514) (AstraZeneca) (Naing et al., J Immunother Cancer. 2019; 7(1):225).

### PD-L1 Antibodies

Antibodies to PD-L1 have been described, for example, in U.S. Pat. Nos. 8,217,149; 8,779,108; 9,637,546; 10,435,470; 10,208,119; 10,689,445; 10,604,581; 10,775,383; 10,759,856; 10,889,648 (incorporated by reference in their entirety).

For example, anti-PD-L1 antibodies have been shown to be effective in human therapy. See, for e.g., atezolizumab (Tecentriq®, Genentech/Roche); avelumab (Bavencio®, Merck and Pfizer); and durvalumab (Imfinzi®, AstraZeneca) which are anti-PD-L1 antibodies approved for use in human therapy of certain cancers.

Additional PD-L1 antibodies in clinical development also are specifically contemplated. For example, KN035 (Zhang et al., Cell Discovery. 3(1): 17004); CK-301 (Checkpoint Therapeutics) (Press release: Checkpoint Therapeutics Initiates Registrational Development Programs for Anti-PD-L1 Antibody CK-301. Source: Checkpoint Therapeutics, Inc.); BMS-936559 (MDX 1105) (Bristol-Myers Squibb/Medarex Inc); Pacmilimab (CX-072) (CytomX Therapeutics) (Guo et al. Front. Immunol., 2020; 11:1508); Sugemalimab (WBP-3155, CS1001) (CStone Pharmaceuticals) (Guo et al. Front. Immunol., 2020; 11:1508); and Cosibelimab (CK-301) (ClinicalTrials.gov Identifier: NCT03212404).

### PD-L2 Antibodies

Antibodies to PD-L2 have been described, for example, in U.S. Pat. Nos. 9,845,356; 10,370,448; and 10,647,771 (incorporated by reference in their entirety). PP71

TABLE PD-A
FDA-approved anti-PD-1/L1 antibodies (adapted from Guo et al. Front.
Immunol., 2020; 11: 1508, incorporated by reference in its entirety).

| | | | Patent number/ | | |
|---|---|---|---|---|---|
| | | | Patent Appl. Pub. | | Antibody |
| Target | Drug | Company | No. | Inventor | class |
| PD-1 | Nivolumab | BMS/Ono | U.S. Pat. No. 7,595,048 | Honjo et al. | IgG₄ |
| | Pembrolizumab | Merck&Co | U.S. Pat. No. 8,952,136 | Carven et al. | IgG₄ |
| | Cemiplimab | Regeneron | U.S. Pat. No. 9,987,500 | Papadopoulos et | IgG₄ |
| | | Pharmaceuticals | | al. | |
| PD-L1 | Avelumab | Merck Serono | US2014341917 | Nastri et al. | IgG₁ |
| | Atezolizumab | Roche | U.S. Pat. No. 8,217,149 | Irving et al. | IgG₁ |
| | Durvalumab | AstraZeneca | U.S. Pat. No. 8,779,108 | Queva et al. | IgG₁ |

Each cite patent is incorporate herein by reference in its entirety, including its description of the aforementioned antibodies and their use.

TABLE PD-B
The patents related to currently developed anti-PD-1/L1 antibodies
(reproduced from Guo et al. Front. Immunol., 2020; 11: 1508).

| | | | Patent No./Patent | | |
|---|---|---|---|---|---|
| Target | Drug | Company | Appl. Pub. No. | Inventor | Antibody class |
| PD-1 | Spartalizumab | Novartis | U.S. Pat. No. 9,683,048B2 | Freeman et al. | IgG₄κ |
| | (PDR-001) | | | | |
| | Cemiplimab | Regeneron | US20150203579 | Papadopoulos | IgG₄ |
| | (Libtayo) | Pharmaceuticals | | et al. | |
| | Camrelizumab | Incyte Biosciences | US20160376367A1 | Yuan et al. | IgG₄ |
| | (SHR-1210) | and | | | |
| | | Jiangsu Hengrui | | | |
| | | Medicine | | | |
| | Tislelizumab | BeiGene | U.S. Pat. No. 8,735,553B1 | Li et al. | IgG₄ |
| | (BGB-A317) | | | | |
| | Dostarlimab | Tesaro/AnaptysBio | U.S. Pat. No. 9,815,897B2 | King et al. | IgG₄ |
| | (TSR-042) | | | | |
| | MEDI-0680 | MedImmune LLC | U.S. Pat. No. 8,609,089B2 | Langermann | IgG₄ |
| | (AMP-514) | | | et al. | |
| | SSI-361 | Lyvgen | US20180346569A1 | Wang et al. | IgG₄ |
| | AMP-224 | Amplimmune Inc | US20130017199 | Langermann | IgG₄ |
| | | | | et al. | |
| PD-L1 | CX-072 | CytomX | US20160311903A1 | West et al. | protease activatable |
| | | | | | prodrug |
| | BMS-936559 | Medarex Inc | U.S. Pat. No. 7,943,743 | Korman et al. | IgG₄ |
| | (MDX 1105) | | | | |
| | KN035 | Jiangsu Alphamab | US20180327494A1 | Xu et al. | fusion protein of |
| | | Biopharmaceuticals | | | humanized |
| | | Co., Ltd. | | | anti-PD-L1 single |
| | | | | | domain |
| | | | | | antibody and human |
| | | | | | IgG₁ Fc |

Each cited patent is incorporated herein by reference in its entirety, including its description of the aforementioned antibodies and their use.

### PD-1/PD-L1 Peptide and Small Molecule Inhibitors

Various PD-1/P-L1 peptide and small inhibitors are contemplated as embodiments. For example, AUNP12 (PD-1/PD-L1 inhibitor) (Aurigene and Laboratories Pierre Fabre) (Juneja et al., 2017 The Journal of Experimental Medicine. 214 (4): 895-904); JTX-4014 (Jounce Therapeutics) (ClinicalTrials Identifier: NCT04549025); CA-170 (PD-L1 and VISTA antagonist) (Okazaki et al., (2006). Trends in Immunology. 27(4): 195-201); BMS-986189 (macrocyclic peptide) (Bristol-Myers Squibb) (“Pharmacokinetics, Safety, Tolerability and Pharmacodynamics of BMS-986189 in Healthy Subjects”. Retrieved clinicaltrials.gov); JQ1 (BET bromodomain inhibitor) (Han et al., Am J Cancer Res 2020; 10(3):727-742); PD-1/PD-L1 macrocyclic peptide (BMSpep-57) (Ganesan et al., Scientific Reports 9, Article number: 12392 (2019); (BMS-103, BMS-142) (Ganesan, et al., Scientific Reports 9, Article number: 12392 (2019); BMS116 (Chen et al., 2020, Oncoimmunology, 9:1, 1831153).

Table PD-C (10): small molecule inhibitors of PD-1 and PD-L1 (reproduced from Guo et al. Front. Immunol., 2020; 11:1508) with associated patent documents, incorporated herein by reference.

TABLE 10
Patents and patent applications of [symbol] [symbol] inventors of [symbol] and [symbol] .

| Type | Target | | Patent number | Inventor |
|---|---|---|---|---|
| [symbol] | [symbol] | [symbol] | [symbol] | [symbol] |
| | [symbol] | [symbol] | [symbol] | [symbol] |
| | | | [symbol] | [symbol] |
| | | | [symbol] | [symbol] |
| | | | [symbol] | [symbol] |
| | | [symbol] | [symbol] | [symbol] |
| | | | [symbol] | [symbol] |
| | | [symbol] | [symbol] | [symbol] |
| | [symbol] | [symbol] | [symbol] | [symbol] |
| | | | [symbol] | [symbol] |
| | | | [symbol] | [symbol] |
| | | [symbol] | [symbol] | [symbol] |
| | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | [symbol] | [symbol] | [symbol] |
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |

[symbol]

indicates data missing or illegible when filed

In some embodiments, the anti-CTLA-4 antibody is a monoclonal antibody directed against the T-cell receptor protein cytotoxic T-lymphocyte-associated protein 4 (CTLA-4). An example of an anti-CTLA-4 antibody is human IgG2 monoclonal antibody tremelimumab which binds to CTLA4 and blocks binding of the antigen presenting cell ligands B7-1 and B7-2 to CTLA-4, resulting in inhibition of B7-CTLA4-mediated downregulation of T-cell activation. Another example of an anti-CTLA-4 antibody is human IgG1 monoclonal antibody ipilimumab which binds to CTLA4 and blocks binding of the antigen presenting cell ligands B7-1 and B7-2 to CTLA-4, resulting in inhibition of B7-CTLA4-mediated downregulation of T-cell activation. Ipilimumab is undergoing clinical trials for the treatment of non-small cell lung carcinoma, small cell lung cancer and metastatic hormone-refractory prostate cancer.

In some embodiments, the anti-GITR antibody is a monoclonal antibody directed against glucocorticoid-induced tumor necrosis factor receptor (GITR) which blocks the interaction of GITR with its ligand, enhances cytotoxicity of natural human killer cells and/or downmodulates GITR expression on peripheral blood lymphocytes.

In some embodiments, the anti-OX40 antibody is an agonistic monoclonal antibody that mimicks the natural OX40 ligand and selectively binds to and activates the OX40 receptor. Receptor activation induces proliferation of memory and effector T cells.

### Exemplary Immune Checkpoint Inhibitor Approved Indications

A summary of indications for various checkpoint inhibitors is provided by Vaddepally R K, et al., “Review of Indications of FDA-Approved Immune Checkpoint Inhibitors per NCCN Guidelines with the Level of Evidence.” Cancers (Basel). 2020 Mar. 20; 12(3):738. doi: 10.3390/cancers12030738. PMID: 32245016; PMCID: PMC7140028, incorporated herein by reference in its entirety.

In some embodiments, cancer patients that do not meet prescribing information, e.g., specified in a product label, such as a government-approved product label, are scored as “insensitive” to a checkpoint inhibitor for purposes of methods of treatment described herein.

Exemplary prescribing information for nivolumab (marketed as OPDIVO®) is provided at https://www.accessdata.fda.gov/drugsatfda_docs/label/2018/125554s0581bl.pdf, incorporated herein by reference in its entirety.

Exemplary prescribing information for Pembrolizumab (marketed as KEYTRUDA®) is provided at https://www.accessdata.fda.gov/drugsatfda_docs/label/2021/125514s0961bl.pdf, incorporated herein by reference in its entirety.

Exemplary prescribing information for Cemiplimab (marketed as LIBTAYO™) is provided at https://www.accessdata.fda.gov/drugsatfda_docs/label/2021/761097s0071bl.pdf, incorporated herein by reference in its entirety.

Exemplary prescribing information for Avelumab (marketed as BAVENCIO®) is provided at https://www.accessdata.fda.gov/drugsatfda_docs/label/2020/761049s0091bl.pdf, incorporated herein by reference in its entirety.

Exemplary prescribing information for Atezolizumab (marketed as TECENTRIQ®) is provided at https://www.accessdata.fda.gov/drugsatfda_docs/label/2021/761034s0421bl.pdf, incorporated herein by reference in its entirety.

Exemplary prescribing information for Durvalumab (marketed as IMFINZI®) is provided at https://www.accessdata.fda.gov/drugsatfda_docs/label/2020/761069s0181b1.pdf, and at https://den8dhaj6zs0e.cloudfront.net/50fd68b9-106b-4550-b5d0-12b045f8b184/9496217c-08b3-432b-ab4f-538d795820bd/9496217c-08b3-432b-ab4f-538d795820bd_viewable_rendition_v.pdf, incorporated herein by reference in its entirety.

For at least some cancers, measurements of checkpoint proteins or ligands are used as a biomarker to identify cancer patients with expected sensitivity to the checkpoint inhibitor. For example, Pembrolizumab is considered:
as a single agent for the first-line treatment of patients with NSCLC expressing PD-L1 [Tumor Proportion Score (TPS)≥1%] as determined by an FDA-approved test, with no EGFR or ALK genomic tumor aberrations;
as a single agent for the first-line treatment of patients with metastatic or with unresectable, recurrent (head and neck squamous cell cancer) HNSCC whose tumors express PD-L1 [Combined Positive Score (CPS)≥1] as determined by an FDA-approved test;
for the treatment of patients with locally advanced or metastatic urothelial carcinoma who are not eligible for cisplatin-containing chemotherapy and whose tumors express PD-L1 [Combined Positive Score (CPS)≥10] as determined by an FDA-approved test;
for the treatment of patients with recurrent locally advanced or metastatic gastric or gastroesophageal junction adenocarcinoma whose tumors express PD-L1 [Combined Positive Score (CPS)≥1] as determined by an FDA-approved test, with disease progression on or after 2 or more prior lines of therapy including fluoropyrimidine- and platinum-containing chemotherapy and if appropriate, HER2/neu-targeted therapy;
for the treatment of patients with locally advanced or metastatic esophageal or gastroesophageal junction (GEJ) (tumors with epicenter 1 to 5 centimeters above the GEJ) carcinoma that is not amenable to surgical resection or definitive chemoradiation as a single agent after one or more prior lines of systemic therapy for patients with tumors of squamous cell histology that express PD-L1 (CPS≥10) as determined by an FDA-approved test;
for the treatment of patients with recurrent or metastatic cervical cancer with disease progression on or after chemotherapy whose tumors express PD-L1 [Combined Positive Score (CPS)≥1] as determined by an FDA-approved test; and
in combination with chemotherapy, for the treatment of patients with locally recurrent unresectable or metastatic triple negative breast cancer (TNBC) whose tumors express PD-L1 [Combined Positive Score (CPS)≥10] as determined by an FDA approved test.

Cemiplimab is considered for the first-line treatment of patients with NSCLC whose tumors have high PD-L1 expression [Tumor Proportion Score (TPS)≥50%] as determined by an FDA-approved test, with no EGFR, ALK or ROS1 aberrations.

Atezolizumab is considered:
for the treatment of adult patients with locally advanced or metastatic urothelial carcinoma who are not eligible for cisplatin-containing chemotherapy and whose tumors express PD-L1 (PD-L1 stained tumor-infiltrating immune cells [IC] covering≥5% of the tumor area), as determined by an FDA-approved test;
as adjuvant treatment following resection and platinum-based chemotherapy for adult patients with Stage II to IIIA NSCLC whose tumors have PD-L1 expression on ≥1% of tumor cells, as determined by an FDA-approved test; and
for the first-line treatment of adult patients with metastatic NSCLC whose tumors have high PD-L1 expression (PD-L1 stained ≥50% of tumor cells [TC≥50%] or PD-L1 stained tumor-infiltrating immune cells [IC] covering ≥10% of the tumor area [IC≥10%]), as determined by an FDA approved test, with no EGFR or ALK genomic tumor aberrations

In some embodiment, the invention uses IMM60 to treat cancer patients to increase PD-L1 expression. In some embodiments, the increased PD-L1 expression converts the patient from ineligible/insensitive (under the prescribing guideline) to a patient classifiable as sensitive (and appropriate for treatment) under the prescribing guideline.

### Checkpoint Inhibitor Biomarker Assays

In some embodiments, the measurement of PD-L1, e.g., for the purpose of evaluating whether a patient satisfies a prescribing guideline, is performed with an immunohistochemistry assay, such as an assay with FDA Premarket Approval (PMA, see https://www.accessdata.fda.gov/) or approval. Exemplary suitable assays are set forth in the following Table:

| | | | Original PMA | Currently Associated |
|---|---|---|---|---|
| Product | Manufacturer | Antibody | Number | Drugs |
| VENTANA PD-L1 (SP263) Assay | Ventana | SP263 | P160046 | TECENTRIQ, LIBTAYO |
| VENTANA PD-L1 (SP142) Assay | Ventana | SP142 | P160002 | TECENTRIQ |
| PD-L1 IHC 28-8 pharmDx | Agilent | 28-8 | P150025/P150027 | OPDIVO |
| PD-L1 IHC 22C3 pharmDx | Agilent | 22C3 | P150013 | KEYTRUDA, LIBTAYO |

There are several scoring methods used across multiple indications and drugs which are summarized from the intended use statements of the products:

TABLE 2

| Name | Assay | Scoring Method | Cutoff | Drug | Indication |
|---|---|---|---|---|---|
| TC | SP263 | Percentage of tumor cells (% TC) with any | ≥1% | TECENTRIQ | NSCLC |
| | | membrane staining above background | ≥50% | LIBAYTO | NSCLC |
| TC | SP142 | Percentage of PD-L1 expressing tumor cells (% | ≥50% | TECENTRIQ | NSCLC |
| | | TC) of any intensity | | | |
| IC | | Proportion of tumor area occupied by PD-L1 | ≥10% | TECENTRIQ | NSCLC |
| | | expressing tumor-infiltrating immune cells (% | | | |
| | | IC) of any intensity | | | |
| Tumor Cell | 28-8 | Percentage of evaluable tumor cells exhibiting | ≥1% | OPDIVO | NSCLC* |
| Expression | | partial or complete membrane staining at any | | | |
| | | intensity | | | |
| Tumor | 22-C3 | Percentage of viable tumor cells showing partial | ≥1% | KEYTRUDA | NSCLC |
| Proportion | | or complete membrane staining at any intensity | | | |
| Score | | | ≥50% | LIBAYTO | NSCLC |
| (TPS) | | | | | |
| Combined | | Number of PD-L1 staining cells (tumor cells, | ≥10% | KEYTRUDA | ESCC, |
| Positive | | lymphocytes, macrophages) divided by the total | | | TNBC |
| Score | | number of viable tumor cells, multiplied by 10 | ≥1% | KEYTRUDA | Cervical |
| (CPS) | | | | | Cancer, |
| | | | | | HNSCC |

*Additional Complementary Diagnostic claims are provided for non-small cell lung cancer, squamous cell carcinoma of the head and neck, and urothelial cancer
Tumor Cell Expression, TC, and Tumor Proportion Score (TPS) are in practice equivalent methods of measuring PD-L1 expression on tumor cells. IC is a measure of only immune cell PD-L1 expression. Combined Positive Score (CPS) takes into account PD-L1 expression on both tumor and immune cells. There was some intellectual property filed around CPS, although it may no longer be in effect.

VENTANA PD-L1 (SP263) Assay is a qualitative immunohistochemistry assay using rabbit monoclonal anti-PDL1 clone SP263 intended for use in the assessment of the programmed death ligand-1 (PD-L1) protein in formalin fixed, paraffin-embedded (FFPE) nonsmall cell lung carcinoma (NSCLC) tissue specimens by light microscopy. The VENTANA PD-L1 (SP263) Assay is used with the OptiView DAB IHC Detection Kit for staining on the BenchMark ULTRA instrument. PD-L1 protein expression in NSCLC is determined by the percentage of tumor cells (% TC) with any membrane staining above background. VENTANA PD-L1 (SP263) Assay is indicated as an aid in identifying patients eligible for treatment with the therapies listed in Ventana Table 1 for the indication and PD-L1 status in accordance with the approved therapeutic product labeling.

TABLE 1
VENTANA PD-L1 (SP263) Assay Companion
Diagnostic Indication.

| Indication | | |
|---|---|---|
| for Use | PD-L1 Cut-off | Therapy |
| NSCLC | ≥1% TC | TECENTRIQ (atezolizumab) |
| | ≥50% TC | LIBTAYO (cemiplimab-rwlc) |

VENTANA PD-L1 (SP142) Assay is a qualitative immunohistochemical assay using rabbit monoclonal anti-PD-L1 clone SP142 intended for use in the assessment of the programmed deathligand 1 (PD-L1) protein in tumor cells and tumor-infiltrating immune cells in the formalin-fixed, paraffin-embedded (FFPE) tissues indicated below stained with OptiView DAB IHC Detection Kit and OptiView Amplification Kit on a BenchMark ULTRA instrument. Determination of PD-L1 status is based on either the proportion of tumor area occupied by PD-L1 expressing tumor-infiltrating immune cells (% IC) of any intensity or the percentage of PD-L1 expressing tumor cells (% TC) of any intensity. VENTANA PD-L1 (SP142) Assay is indicated as an aid for identifying patients for treatment with the therapy- and cutoff listed in Table 1 in accordance with the approved therapeutic product labeling.

TABLE 1
Companion diagnostic indications for
the VENTANA PD-L1 (SP142) Assay.

| Indications for use | Therapy | Cutoff |
|---|---|---|
| Non-Small Cell Lung | TECENTRIQ | ≥50% TC or ≥10% IC |
| Cancer (NSCLC) | | |

PD-L1 IHC 28-8 pharmDx is a qualitative immunohistochemical assay using Monoclonal Rabbit Anti-PD-L1, Clone 28-8 intended for use in the detection of PD-L1 protein in formalin-fixed, paraffin-embedded (FFPE) non-small cell lung cancer (NSCLC), squamous cell carcinoma of the head and neck (SCCHN), and urothelial carcinoma (UC) tissues using EnVision FLEX visualization system on Autostainer Link 48.

PD-L1 protein expression is defined as the percentage of evaluable tumor cells exhibiting partial or complete membrane staining at any intensity.

Companion Diagnostic Indication

| | PD-L1 Expression | |
|---|---|---|
| Tumor Indication | Clinical Cutoff | Intended Use |
| NSCLC | ≥1% tumor cell | PD-L1 IHC 28-8 pharmDx is indicated as an aid in identifying NSCLC |
| | expression | patients for treatment with OPDIVO ® (nivolumab) in combination with |
| | | YERVOY ® (ipilimumab). |

When used in accordance with approved therapeutic labeling:
PD-L1 expression (100 or 500 or 10% tumor cell expression), as detected by PD-L1 JHC 28-8 pharmDx in non-squamous NSCLC (nsNSCLC) may be associated with enhanced survival from OPDiVO®;
PD-L1 expression (≥1% tumor cell expression), as detected by PD-L1 JHC 28-8 pharmDx in SCCHN may be associated with enhanced survival from OPDIVO®;
PD-L1 expression (≥1% tumor cell expression), as detected by PD-L1 IHC 28-8 pharmDx in UC may be associated with enhanced response rate and enhanced disease-free survival from OPDIVO®.

PD-L1 IHC 22C3 pharmDx is a qualitative immunohistochemical assay using monoclonal mouse anti-PD-L1, Clone 22C3 intended for use in the detection of PD-L1 protein in formalin-fixed, paraffin-embedded (FFPE) non-small cell lung cancer (NSCLC), esophageal squamous cell carcinoma (ESCC), cervical cancer, head and neck squamous cell carcinoma (HNSCC), and triple-negative breast cancer (TNBC) tissues using EnVision FLEX visualization system on Autostainer Link 48.

PD-L1 protein expression in NSCLC is determined by using Tumor Proportion Score (TPS), which is the percentage of viable tumor cells showing partial or complete membrane staining at any intensity.

PD-L1 protein expression in ESCC, cervical cancer, HNSCC, and TNBC is determined by using Combined Positive Score (CPS), which is the number of PD-L1 staining cells (tumor cells, lymphocytes, macrophages) divided by the total number of viable tumor cells, multiplied by 100.

PD-L1 IHC 22C3 pharmDx is indicated as an aid in identifying patients for treatment with the therapies for the indications listed in PD-L1 IHC 22C3 Table 1.

TABLE 1
PD-L1 IHC 22C3 pharmDx companion diagnostic indications,
PD-L1 expression levels, and therapies

| Tumor Indication | PD-L1 Expression Level | Therapy |
|---|---|---|
| NSCLC | TPS ≥1% | KEYTRUDA ®* |
| ESCC | CPS ≥10 | |
| Cervical Cancer | CPS ≥1 | |
| HNSCC | CPS ≥1 | |
| TNBC | CPS ≥10 | |
| NSCLC | TPS ≥50% | LIBTAYO ®** |

*See KEYTRUDA ® product label for specific clinical circumstances guiding PD-L1 testing.
**See the LIBTAYO ® product loyal for specific clinical circumstances guiding PD-L1 testing.

### Cytokines

Cytokines that are effective in inhibiting tumor growth/metastasis are contemplated for use in the combination therapy. Such cytokines, lymphokines, or other hematopoietic factors include, but are not limited to, M-CSF, GM-CSF, TNF, IL-1, IL-2, IL-3, IL-4, IL-5, IL-6, IL-7, IL-8, TL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin.

### Cancer Vaccines and Tumor Antigens

An immunotherapeutic agent can be a cancer vaccine. A cancer vaccine is an agent, molecule, or immunogen which stimulates or elicits an endogenous immune response in an individual or subject against one or more tumor antigens.

As used herein, a cancer antigen is broadly defined as an antigen specifically expressed by a tumour or cancer cell: a cancer antigen which is present on the surface of cancer cells in an individual, but which is not present on the surface of normal somatic cells of the individual, i.e., the antigen is exposed to the immune system in cancer cells but not in normal somatic cells. The antigen may be expressed at the cell surface of the tumour cell where it is recognized by components of the humoral immune system such as B lymphocytes (B cells). Intracellular tumour antigens are processed into shorter peptide fragments which form complexes with major histocompatibility complex (MHC) molecules and are presented on the cell surface of cancer cells, where they are recognized by the T cell receptors (TCR's) of T lymphocytes (T cells). Preferably, the cancer antigen is one which is not expressed by normal cells, or at least not expressed to the same level as in tumour cells. An immunotherapeutic agent, such as a cancer vaccine maybe comprised of one or more epitopes or antigenic determinants, e.g., peptide epitopes or antigenic determinants from a tumor or cancer antigen, such that the immune response generated by the cancer vaccine is reactive against the antigen.

A cancer vaccine may enhance the presentation of one or more cancer antigens to both antigen-presenting cells (e.g., macrophages and dendritic cells) and/or to other immune cells such as T cells, B cells, and NK cells. In some examples, preparations and/or formulations of cancer vaccines may be used together with one or more adjuvants that are well known in the art, to induce an immune response or to increase an immune response.

Cancer antigens may include, for example, cancer-testis antigens encoded by cancer-germ line genes. Cancer-testis (CT) antigens constitute a unique group of genes which are predominantly expressed in human germ line cells such as placenta and testis but become reactivated in various malignancies (Simpson et al., Nature Rev (2005) 5, 615-625). Most of these genes are located as multigene families on the X-chromosome and are also referred to as CT-X antigens (Simpson et al., Nature Rev (2005) 5, 615-625). Analogies have been drawn between their expression pattern during germ maturation and neoplastic transformation, thus suggesting their involvement in several steps of tumorigenesis (Simpson et al., Nature Rev (2005) 5, 615-625). The CT-X antigens are broadly expressed in a wide variety of cancer types including for example bladder cancer, lung cancer, ovarian cancer, breast cancer, prostate cancer, Brain cancer, glioma, glioblastoma, hepatocellular carcinoma and melanoma. Moreover, their expression pattern is closely associated with advanced disease and poor outcome and might thus be of diagnostic and/or prognostic relevance (Gure et al., Clin Cancer Res (2005) 11, 8055-8062; Velazquez et al., Cancer Immun. (2007) 7(1):11; Andrade et al., Cancer Immun (2008) 8(1):2; Tinguely et al., Cancer Science (2008) 99(4):720-25; Napoletano et al., Am. J. of Obstet. Gyn. (2008) 198:99 e91-97). Due to their highly restricted expression in malignant tissues, their tumour associated peptide epitopes provide promising targets for anticancer immunotherapy (Scanlan et al., Immunol Rev (2002) 188, 22-32). Indeed, clinical trials evaluating the role of CT antigens, namely MAGE-A3, Prame and NY-ESO-I, as targets for specific immunotherapy have already been initiated in a number of different malignancies (Bender et al., Cancer Immunol (2007) 7, 16; Atanackovic et al., PNAS (2008) 105, 1650-1655; Jager et al., PNAS (2006) 103, 14453-14458; van Baren et al., J Clin Oncol (2005) 23, 9008-9021; Valmori et al., PNAS (2007) 104, 8947-8952; Odunsi et al., PNAS (2007) 104, 12837-12842; Davis et al., PNAS (2004) 101, 10697-10702 (9-15). Tumor antigens which may be comprised of the full-length polypeptide sequence of the tumor antigen or an immunogenic fragment, or epitope derived from the full-length polypeptide sequence of the tumor antigen. Tumor antigens include the corresponding nucleotide sequence encoding for the full-length polypeptide, immunogenic fragment, or epitope derived from the full-length polypeptide sequence of the tumor antigen.

A fragment of a cancer antigen is a contiguous stretch of amino acid residues from the sequence of the antigen which is shorter than the full-length antigen (i.e., it consists of fewer amino acid residues). For example, a fragment may comprise less than 500, less than 400, less than 300, less than 200, less than 100 amino acids, or less than 50 amino acids. A fragment will generally consist of at least 5 amino acids, for example, at least 10 amino acids, at least 15 amino acids, at least 20 amino acids, at least 25 amino acids, at least 30 amino acids or at least 35 amino acids. Fragments of cancer antigens may include immunogenic regions or epitopes that bind to MHC class I or class II molecules and are recognized by TCR's of T lymphocytes. Many such epitopes of cancer antigens are known in the art, such as listed on the webpage www.cancerimmunity.org/peptidedatabase/Tcellepitopes.

The cancer antigen can be a tumor associated peptide, or protein that induces or enhances immune response and is derived from tumor associated genes and encoded proteins including, for example, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2(HOM-MEL-40), SSX-1, SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomeras, GnTV, Herv-K-mel, Lage-1, Mage-C2, NA-88, /Lage-2, SP17, and TRP2-Int2, (MART-I), gp100 (Pmel 17), TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens Eand E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, .beta.-Catenin, CDK4, Mum-1, p16, TAGE, PSMA, PSCA, CT7, telomerase, 43-9F, 5T4, 791Tgp72, .alpha.-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS. For example, antigenic peptides characteristic of tumors include those listed in International Patent Application Publication No. WO 2000/020581 and U.S. Patent Application Publication No. 2010/0284965, which are each incorporated herein by reference. In some embodiments, the antigen is a tumor antigen selected from the group consisting of MUC1. MAGE, BAGE, RAGE, CAGE, SSX-2, NY-ESO-1, PRAME, PSMA, tyrosinase, melan-A, and mixtures thereof. In some variations, the cancer antigen is a mammalian protein. In some variations, the cancer antigen is a human protein. In some variations, the full-length protein is employed as the antigen. In some variations, peptides comprising an antigenic fragment of these proteins is used as the tumor antigen.

Other suitable antigens include cancer antigens in the following classes: cancer testis antigens (e.g., HOM-MEL-40), differentiation antigens (e.g., HOM-MEL-55), overexpressed gene products (HOM-MD-21), mutated gene products (NY-COL-2), splice variants (HOM-MD-397), gene amplification products (HOM-NSCLC-11) and cancer related autoantigens (HOM-MEL-2.4) as reviewed in Cancer Vaccines and Immunotherapy (2000) Eds Stern, Beverley and Carroll, Cambridge University Press, Cambridge. Further examples include, MART-1 (Melanoma Antigen Recognized by T-cells-1) MAGE-A (MAGE-A1, MAGE-A2, MAGE-A3, MAGE-A4, MAGE-A6, MAGE-A8, MAGE-A10, MAGE-A12), MAGE B (MAGE-B1-MAGE-B24), MAGE-C (MAGE-C1/CT7, CT10), GAGE (GAGE-1, GAGE-8, PAGE-1, PAGE-4, XAGE-1, XAGE-3), LAGE (LAGE-1a(1S), -1b(1L), NY-ESO-1), SSX (SSX1-SSX-5), BAGE, SCP-1, PRAME (MAPE), SART-1, SART-3, CTp11, TSP50, CT9/BRDT, gp100, MART-1, TRP-1, TRP-2, MELAN-A/MART-1, Carcinoembryonic antigen (CEA), prostate-specific antigen (PSA), MUCIN (MUC-1) and Tyrosinase. TAAs are reviewed in Cancer Immunology (2001) Kluwer Academic Publishers, The Netherlands. Additional cancer associated antigens include Her 2, survivin and TERT. In some embodiments, the tumor antigen comprises NY-ESO-1 or an antigenic fragment thereof.

The term “antigen” refers to protein or peptide to be introduced into a subject. As described herein, an antigen may be provided through delivering a peptide or protein or through delivering a nucleic acid encoding a peptide or protein.

By “antigen” in the context of the present disclosure it is also meant to incorporate an antigenic peptide derived from an antigen. In particular, “cancer associated antigen” is intended to encompass a peptide derived from a cancer associated antigen.

An antigen such as a cancer-associated antigen can be provided for use as a medicament in several different ways. It can be administered as part of a vector.

Any suitable vector may be used to introduce a polynucleotide that encodes a polypeptide of the invention encoding one of the tumor antigen proteins into the host. Additional vectors that have been described in the literature include replication deficient retroviral vectors, including but not limited to lentivirus vectors [Kim et al., J. Virol., 72(1): 811-81(1998); Kingsman & Johnson, Scrip Magazine, October, 1998, pp. 43 46.]; adeno-associated viral (AAV) vectors [U.S. Pat. Nos. 5,474,935; 5,139,941; 5,622,856; 5,658,776; 5,773,289; 5,789,390; 5,834,441; 5,863,541; 5,851,521; 5,252,479; Gnatenko et al., J. Invest. Med., 45: 87 98 (1997)]; adenoviral (AV) vectors [See, e.g., U.S. Pat. Nos. 5,792,453; 5,824,544; 5,707,618; 5,693,509; 5,670,488; 5,585,362; Quantin et al., Proc. Natl. Acad. Sci. USA, 89: 2581 2584 (1992); Stratford Perricadet et al., J. Clin. Invest., 90: 62630 (1992); and Rosenfeld et al., Cell, 68: 143 155 (1992)]; an adenoviral adenoassociated viral chimeric (see for example, U.S. Pat. No. 5,856,152) or a vaccinia viral or a herpesviral (see for example, U.S. Pat. Nos. 5,879,934; 5,849,571; 5,830,727; 5,661,033; 5,328,688; Lipofectin mediated gene transfer (BRL); liposomal vectors [See, e.g., U.S. Pat. No. 5,631,237 (Liposomes comprising Sendai virus proteins)]; and combinations thereof.

Suitable cancer vaccines are known in the art and may be produced by any convenient technique.

For example, a cancer vaccine may be generated wholly or partly by chemical synthesis. For example, a peptide-based vaccine or immunogen may be synthesised using liquid or solid-phase synthesis methods; in solution; or by any combination of solid-phase, liquid phase and solution chemistry, e.g., by first completing the respective peptide portion and then, if desired and appropriate, after removal of any protecting groups being present, by introduction of the residue X by reaction of the respective carbonic or sulfonic acid or a reactive derivative thereof. Chemical synthesis of peptides is well-known in the art (J. M. Stewart and J. D. Young, Solid Phase Peptide Synthesis, 2nd edition, Pierce Chemical Company, Rockford, Illinois (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A Users Manual, ABI Inc., Foster City, California; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

Alternatively, peptide-based cancer vaccines may be generated wholly or partly by recombinant techniques. For example, a nucleic acid encoding a cancer antigen may be expressed in a host cell and the expressed antigen isolated and/or purified from the cell culture. For example, antigen may be expressed in E. coli either in soluble form or in inclusion bodies, which may be solubilized and refolded. After expression, the antigen may be isolated and/or purified. Cancer antigen may be analyzed by standard techniques, such as mass spectrometry and western blot analysis.

The use of tumor antigens to generate immune responses is well-established in the art (see for example: Kakimi K, et al. Int J Cancer. 2011 Feb. 3; Kawada J, Int J Cancer. 2011 Mar. 16; Gnjatic S, et al. Clin Cancer Res. 2009 Mar. 15; 15(6):2130-9; Yuan J, et al. Proc Natl Acad Sci USA. 2008 Dec. 23; 105(51):20410-5; Sharma P, et al. J Immunother. 2008 Nov.-Dec. 31(9):849-57; Wada H, et al. Int J Cancer. 2008 Nov. 15; 123(10):2362-9; Diefenbach C S, et al. Clin Cancer Res. 2008 May 1; 14(9):2740-8; Bender A, et al. Cancer Immun. 2007 Oct. 19; 7:16; Odunsi K, et al. Proc Natl Acad Sci USA. 2007 Jul. 31; 104(31):12837-42; Valmori D, et al. Proc Natl Acad Sci USA. 2007 May 22; 104(21):8947-52; Uenaka A, et al. Cancer Immun. 2007 Apr. 19; 7:9; Kawabata R, et al. Int J Cancer. 2007 May 15; 120(10):2178-84; Jager E, et al. Proc Natl Acad Sci USA. 200 September 26; 103(39):14453-8; Davis ID Proc Natl Acad Sci USA. 2005 Jul. 5; 102(27):9734; Chen Q, Proc Natl Acad Sci USA. 2004 Jun. 22; 101(25):9363-8; Jager E, Proc Natl Acad Sci USA. 2000 Oct. 24; 97(22):12198-203; Carrasco J, et al. J Immunol. 2008 Mar. 1; 180(5):3585-93; van Baren N, et al. J Clin Oncol. 2005 Dec. 10; 23(35):9008-21; Kruit W H, et al. Int J Cancer. 2005 Nov. 20; 117(4):596-604; Marchand M, et al. Eur J Cancer. 2003 January; 39(1):70-7; Marchand M et al. Int J Cancer. 1999 Jan. 18; 80(2):219-30; Atanackovic D, et al. Proc Natl Acad Sci USA. 2008 Feb. 5; 105(5):1650-5).

Typically, an immunotherapeutic agent, such as a cancer vaccine, is administered to the individual whose cancer expresses the said antigen. Cancer cells from the individual may be analyzed to identify the cancer antigen and patients are then identified for administration of the appropriate immunotherapeutic agent or cancer vaccine. For example, a method as described herein may comprise the step of identifying a cancer antigen which is displayed by one or more cancer cells in a sample obtained from the individual.

A biological sample may be obtained from the subject such as a biopsy, blood or bone marrow sample and tested for the presents of cancer cells which may be identified as displaying the cancer antigen using any standard techniques including but not limited to immunological techniques, such as immunocytochemistry and immunohistochemistry may be employed. Additional techniques include immunological analysis such as serologically determining an autologous immune response to said cancer antigen, see WO2001/007917. Analysis of gene expression can be performed using methods known in the art such as polymerase chain reaction or microarray analysis.

The cancer vaccine may be administered in conjunction with an adjuvant, including adjuvants described above. Additional suitable adjuvants include aluminum salts, such as alum (aluminium potassium sulphate dodecahydrate), aluminum hydroxide and aluminum phosphate and organic compounds, such as squalene.

In addition to a cancer antigen, an immunotherapeutic, immunogenic or vaccine formulation may comprise an adjuvant. For example, a formulation may comprise 1-500 μg, preferably 1-50 μg, of cancer antigen and 0.5 to 20 mg, preferably 1-10 mg, of adjuvant in a pharmaceutically acceptable carrier or diluent as mentioned above.

A vaccine formulation may comprise a Toll-like Receptor (TLR) ligand. Suitable TLR ligands include polyinosinicpolycytidylic acid (poly I:C), lipopolysaccharide (LPS), CpG oligodeoxynucleotide, poly LC, poly ICLC, MPL (Corixa Corp) and imidazoquinolines, such as imiquimod and R848. The use of TLR ligands to modulate immune responses is well-known in the art (see for example, Weiner et al (1997) PNAS USA 94 10833-10837; Vabulas et al J. Immunol. (2000) 164 2372-2378; Gunzer et al (2005) Blood 102424-2432).

Formulations of immunotherapeutic agents, such as cancer vaccines, are well-known in the art and include MAGE-A3 ASCI, NY-ESO-1 ASCI and PRAME ASCI (GSK Bio); Provenge (Dendreon), Abogovomab (Meranini), M-Vax (Avax), Allovectin-7 (Vial) for metastatic melanoma, GSK1572932A (GSK Bio) Belagenpumatucel-L (Novarex) BMP-25 (Merck Serono), BiovaxID (Biovest/Accentia), MDX-1379 (Medarex/BMS), Ipilimumab (BMS) Trovax (Oxford Biomedical) Oncophage (Antigenics) and PR1 leukemia peptide (The Vaccine company).

### Antibody Co-Therapeutics

Embodiments that involve use of antibodies as co-therapeutics may include all varieties of antibodies that have been developed for such purposes, including but not limited to monoclonal antibodies, chimeric antibodies, and antibody fragments.

“Monoclonal antibody” refers to an antibody obtained from a population of substantially homogeneous antibodies. Monoclonal antibodies are generally highly specific, and may be directed against a single antigenic site, in contrast to polyclonal antibody preparations that typically include different antibodies directed against the same or different determinants (epitopes). In addition to their specificity, monoclonal antibodies are advantageous in that they are synthesized by the homogeneous culture, uncontaminated by other immunoglobulins with different specificities and characteristics.

Monoclonal antibodies may be made by the hybridoma method first described by Kohler et al. (Nature, 256:495-7, 1975) (Harlow & Lane; Antibodies: A Laboratory Manual, Cold Spring Harbor Laboratory Press: Cold Spring Harbor, New York (1988); Goding, Monoclonal Antibodies: Principles and Practice, pp. 59-103 (Academic Press, 1986), or may be made by recombinant DNA methods (see, e.g., U.S. Pat. No. 4,816,567). The monoclonal antibodies may also be isolated from phage antibody libraries using the techniques described in, for example, Clackson et al., (Nature 352:624-628, 1991) and Marks et al., (J. Mol. Biol. 222:581-597, 1991). Additional methods for producing monoclonal antibodies are well-known to a person of ordinary skill in the art.

Monoclonal antibodies, such as those produced by the above methods, are suitably separated from culture medium, ascites fluid, or serum by conventional immunoglobulin purification procedures such as, for example, protein A-Sepharose, hydrophobic interaction chromatography (HIC), ion exchange chromatography, hydroxyapatite chromatography, gel electrophoresis, dialysis, and/or affinity chromatography.

It is further contemplated that antibodies of the present disclosure may be used as smaller antigen binding fragments of the antibody that are well-known in the art and described herein.

Because chimeric or humanized antibodies are less immunogenic in humans than the parental non-human (e.g., mouse) monoclonal antibodies, they can be used for the treatment of humans with far less risk of anaphylaxis.

Chimeric monoclonal antibodies, in which the variable Ig domains of a non-human (e.g., mouse) monoclonal antibody are fused to human constant Ig domains, can be generated using standard procedures known in the art (See Morrison et al., Proc. Natl. Acad. Sci. USA 81, 6841-6855 (1984); and Boulianne et al, Nature 312, 643-646, (1984)).

Humanized antibodies may be achieved by a variety of methods including, for example: (1) grafting the non-human complementarity determining regions (CDRs) onto a human framework and constant region (a process referred to in the art as humanizing through “CDR grafting”), (2) transplanting the entire non-human variable domains, but “cloaking” them with a human-like surface by replacement of surface residues (a process referred to in the art as “veneering”), or, alternatively, (3) substituting human amino acids at positions determined to be unlikely to adversely affect either antigen binding or protein folding, but likely to reduce immunogenicity in a human environment (e.g., HUMAN ENGINEERING™). In the present disclosure, humanized antibodies will include both “humanized,” “veneered” and “HUMAN ENGINEERED™” antibodies. These methods are disclosed in, e.g., Jones et al., Nature 321:522 525 (1986); Morrison et al., Proc. Natl. Acad. Sci., U.S.A., 81:6851-6855 (1984); Morrison and Oi, Adv. Immunol., 44:65-92 (1988); Verhoeyer et al., Science 239:1534-153(1988); Padlan, Molec. Immun. 28:489-498 (1991); Padlan, Molec. Immunol. 31:169-217 (1994); Studnicka et al. U.S. Pat. No. 5,766,886; Studnicka et al., (Protein Engineering 7: 805-814, 1994; Co et al., J. Immunol. 152, 2968-297(1994); Riechmann, et al., Nature 332:323-27 (1988); and Kettleborough et al., Protein Eng. 4:773-783 (1991) each of which is incorporated herein by reference. CDR grafting techniques are known in the field, see for example, Riechmann, et al. (Nature 332:323-27 (1988)).

Human antibodies to target protein can also be produced using transgenic animals that have no endogenous immunoglobulin production and are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/0090 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 and U.S. Pat. No. 6,091,001 disclose the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. See also, U.S. Pat. Nos. 6,114,598 6,657,103 and 6,833,268; Green L L, Curr Drug Discovery Technol., 11(1), 74-84, 2014; Lee E C et al., Nature Biotechnology, 32:356-363, 2014; Lee E C and Owen M, Methods Mol Biol., 901:137-48, 2012).

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

IMM60 is a highly lipophilic molecule (necessary for its tight binding to CD1d) and is practically insoluble in aqueous solutions. Thus, a liposomal formulation was selected so that IMM60 can be delivered in a physically stable and soluble form necessary for the IV route of administration. A related physiochemical consideration is that IMM60 has a high calculated log of the partition coefficient (cLogP) value (17.6), indicating its lipophilicity. Structurally, IMM60 contains 2 straight chain aliphatic tails. IMM60 is more lipophilic based on the cLogP than DSPC. Therefore, IMM60—with its long lipid chain—is intercalated within the liposomal bilayer and solubilized as a component of the phospholipid bilayer membrane. Liposomal formulations can specifically target the tumor site through the enhanced permeability and retention (EPR) effect. Micelles and liposomes containing anticancer drugs are some examples of nanoparticle formulations in which small, colloidal nanoparticles, in the range of 100 to 200 nm, are able to extravasate across tumour vasculature, and penetrate and accumulate in tumor tissue via the EPR effect. Additionally, the drug concentration in the tumor compared to the blood can be as high as 10 to 30 times when administered in liposomes versus solubilized formulations, and the EPR effect can be evident within 10 minutes after IV injection. Given these physicochemical properties of IMM60 and these practical advantages of liposomes to target tumors, it is envisioned to be an ideal vehicle for a glycolipid such as IMM60 for use in the treatment of cancer.

### Example 1—Preparation of Compositions

### Materials and Methods

API/lipid solvent solution (alcohol solution): The API was weighed and dissolved in water miscible organic solvent system at temperature, typically 15 minutes of mixing was required to fully dissolve the API at 60° C. The dissolved API is added to the lipids. To complete dissolution of the lipids in solvent, water may be added to ensure complete dissolution of all the lipids. Placebo batches did not include the API in the solvent solution.

Lipid Hydration (MLV formation): The aqueous phase is composed of 145 mM sodium chloride and 10 mM sodium phosphate at pH 6.5 or an alternative buffer. Lipids are hydrated and multilamellar vesicles (MLVs) are formed upon dilution of the organic phase containing the API and lipid into the aqueous phase. Hydration is allowed to proceed for a few minutes at temperature with mixing.

Extrusion: The Lipex extrusion system is set up, connected to a heated water bath to maintain the process temperature, and prepared with several layers of polycarbonate filters. MLVs are put through a LIPEX extruder with polycarbonate filters to perform size reduction to large unilamellar vessicles (LUVs) with a target vesicle size in the range between 90 and 100 nm, in diameter. Nitrogen gas is used to pressurize the system up to 600 psi and to extrude the MLVs through the polycarbonate membrane filters. Several extrusion passes may be required to attain the target particle size range.

Diafiltration: Upon size reduction the LUVs are cooled to near room temperature (<30° C.) and is loaded onto diafiltration cartridges. In this process the liposomes are essentially washed using tangential flow with buffer exchange using 145 mM sodium chloride and 10 mM sodium phosphate at pH 6.5 to remove organic solvent from the formulation. Tangential flow filtration is also used to concentrate the product to adjust the lipid and API concentration to the target specification (to 1 mg/mL API and 9 mg/ml lipids).

Clarifying and Sterile Filtration: Upon completion of the diafiltration process the product is filtered through a sterilizing grade 0.2 μm filter to reduce bioburden. Ideally, the concentration of the product is slightly at higher value than label claim. Following clarifying filtration, the product is stored overnight during in process determination for lipid and API content. The final concentration of the product is diluted to label claim and sterile filtered aseptically by redundant filtration 2×0.2 μm, mPES membrane filters (Sartopore, Sartorius).

Particle Size Measurements by Dynamic Light Scattering: During manufacturing the particle size was monitored using dynamic light scattering measurements at two angles, 900 and 12.8°. The product was diluted to a lipid concentration range between 0.5 mg/mL and 1 mg/mL in saline (0.9% sodium chloride) as diluent, pre-equilibrated for 2 minutes at 23° C. in disposable sizing cuvettes and read using the Malvern Zetasizer instrument with the following settings: dispersant refractive index (RI) of 1.332, material RI of 1.50.

Preparing liposomes as disclosed herein. An alcohol solution (e.g., 4.5 mL ethanol and 0.5 mL water) including the lipids and API (e.g., 50 mg/mL lipid; 5.2 mg/mL API) was prepared and heated to 65° C. while stirring. For example, 4.5 mL of ethanol/0.5 mL of water were charged with 0.1238 g DSPC, 0.1004 g DSPG, and 0.0272 g IMM60 (API required: 0.0259 g corrected for 95.3% purity). The alcohol solution was added to a buffer solution (e.g., 250 mM sucrose and 145 mM NaCl). For example, 5 mL of the alcohol solution was added to 45 mL of buffer solution heated at 65° C. while stirring for a minimum of 5 minutes ([lipid]=4.5 mg/mL, [API]=0.5 mg/mL, solvent=9%) to form 50 mL of a solution of multi-lamellar vesicles. The multi-lamellar vesicles solution was extruded, e.g., through 2×80 nm membranes at 65° C., to form the liposomes. The liposomes were immediately diluted with an equal volume of room temperature buffer and ultrafiltered until concentrated to a volume of 50 mL ([API]=0.5 mg/mL). The ultrafiltered solution was diafiltered (e.g., Krosflow) with 50 mL reservoir (e.g., 115 cm²) against 10 working volumes (500 mL). The solution was concentrated down to about 1.3 mg/mL of API. The solution was filtered using a clarifying filter at ambient temperature (filter 0.2 m Sartopore (mPES membrane). The filtered solution is then diluted to the label claim of 1 mg/mL of API with buffer. Then the solution was sterile filtered through redundant 0.2 m Sartopore (mPES membrane) sterile filters. The estimated yield was about 85%.

The alcohol solution above can contain two lipids selected from the group of:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); and,
(f) 1,2-dimyristoyl-sn-glycero-3-phosphoglycerol (DMPG) and cholest-5-en-30-ol (CHOL).
(g) 1,2-distearoyl-sn-glycero-3-phosphocholine (DSPC) and 1,2-dipalmitoyl-sn-glycero-[phosphor-rac-(1-glycerol)](DPPG); and
(h) 1,2-distearoyl-sn-glycero-3-phospho-rac-glycerol (DSPG) and 1,2-dipalmitoyl-sn-glycero-[phosphor-rac-(1-glycerol)](DPPG).

In some embodiments, the alcohol solution may comprise at least two (e.g., 3) phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG). In some embodiments, the alcohol solution consist essentially of DSPC and DSPG.Development of Formulations: The first liposomal IMM60 prototypes were prepared in studies A, B, C and D according to Table 1. Table 2 and Table 3 summarize their particle size and API and lipid content after sterile filtration. Initially, a liposomal IMM60 prototype was prepared to assess feasibility of the manufacturing process by incorporating IMM60 into EPC/EPG liposomes (A). The particle size of the MLV from batch A was particularly small with ~5 nm and remained stable throughout the process. API content was below the 1 mg/mL target (refer to Table 3). The low API content in the final product was attributed to dilution of product and not loss of API during diafiltration.

Subsequently, a second batch in which IMM60 was incorporated into DSPC/DSPG liposomes was manufactured (B). In contrast to A, the particle size of the MLVs from batch B was large with ~178 nm. Although extrusion resulted in size reduction, the particle size of the LUVs post-ultrafiltration/diafiltration was slightly large (>100 nm). Sterile filtration resulted in removal of some particles as evidenced by the decrease in particle size (~101 nm). Correspondingly, the recovery of API—and lipid— was low (refer to Table 3).

Seven additional liposomal IMM60 prototypes were prepared by incorporating IMM60 into various formulations (C01 to C07), as outlined in Table 1. Of the seven different formulations manufactured in C, batches 01-03 and 05-0 were to be successfully manufactured. Prototype C01 resulted in larger particle size (68 nm) and better API incorporation (0.957 mg/mL) relative to the first prototype (A) with the same API and lipid composition (EPC/EPG/IMM60; weight ratio 2.25/6.75/1). Prototype C02 (POPC, DDAB, IMM60) and C03 (POPC, DC-Chol, IMM60) showed >70% API incorporation and performed well during manufacturing albeit long DF process time for prototype C02. Batch 05 (DSPC/DSPG) did exhibit some particle size growth and was difficult to sterile-filter, resulting in a loss of 50% of the API and lipids. Batches 04 and 07 contained DOTAP which appeared to significantly degrade during the solubilisation process, as the DOTAP content was well below expectations in the MLV's (roughly 0.1-0.2 mg/mL). The degradation may have been due to the high temperature (60° C.) that was required in the manufacturing process, as the API solubility is dependent on this temperature. Batch 04 contained POPC/DOTAP and exhibited slight particle size growth. Batch 07 contained only cholesterol as the second lipid and significantly increased in size. Batch 07 also showed flocculation, which lead to the difficulty in filtration and the significant loss of lipids and API.

The DOTAP:POPC batch was manufactured a second time under protocol D with a shorter temperature exposure time and a sucrose buffer instead of saline. This repeated batch showed improvements in particle size, as well as increased stability of lipids during each stage of the manufacturing process.

TABLE 1
API and Lipid Composition

| Batch | Composition (wt/wt/wt) |
|---|---|
| A | EPC, EPG, IMM60 (2.25/6.75/1) |
| B | DSPC, DSPG, IMM60 (4.78/3.88/1) |
| C01 | EPC, EPG, IMM60 (2.25/6.75/1) |
| C02 | POPC, DDAB, IMM60 (4.58/3.04/1) |
| C03 | POPC, DC-Chol, IMM60 (4.60/2.60/1) |
| C04 | POPC, DOTAP, IMM60 (4.61/3.40/1) |
| C05 | DSPC, DSPG, IMM60 (4.76/3.86/1) |
| C06 | DMPG, Chol, IMM60 (4.60/1.64/1) |
| C07 | DOTAP, Chol, IMM60 (4.66/1.64/1) |
| D01 | POPC, DOTAP, IMM60 (4.61/3.40/1) |

TABLE 2
Particle Size Data

| | |
|---|---|
| 90° | 12.8° |

| Batch | Size (nm) | Pdl | Size (nm) | Pdl |
|---|---|---|---|---|
| A | 52.15 | 0.082 | 48.47 | 0.049 |
| B | 101.80 | 0.122 | 127.60 | 0.222 |
| C01 | 68.67 | 0.098 | 73.27 | 0.162 |
| C02 | 83.17 | 0.059 | 93.88 | 0.174 |
| C03 | 92.30 | 0.109 | 132.9 | 0.320 |
| C04 | 107.8 | 0.061 | 116.4 | 0.203 |
| C05 | 101.6 | 0.107 | 138.9 | 0.229 |
| C06 | 101.5 | 0.144 | 116.5 | 0.152 |
| C07 | 234.2 | 0.218 | 602.8 | 0.858 |
| D01 | 51.04 | 0.252 | 83.93 | 0.659 |

TABLE 3

| | | | Drug to Lipid |
|---|---|---|---|
| Batch | Total Lipid (mg/mL) | IMM60 (mg/mL) | Ratio (w/w) |
| A | 5.20 | 0.706 | 0.136 |
| B | 5.97 | 0.641 | 0.115 |
| C01 | Present | 0.957 | NApp |
| C02 | 6.77 | 0.713 | 0.105 |
| C03 | 8.25 | 0.886 | 0.107 |
| C04 | 5.84 | 0.981 | 0.168 |
| C05 | 5.72 | 0.473 | 0.083 |
| C06 | 6.20 | 0.924 | 0.149 |
| C07 | 0.81 | 0.340 | 0.420 |
| D01 | 7.52 | 0.905 | 0.120 |

From all of the prototypes prepared, the lead formulation (C05) was chosen based on an efficacy study, despite its suboptimal sterile filtration. The manufacturing process for this prototype was optimized in study E, as final sterile filtration of the product was found to be challenging. The filtration step resulted in filter clogging and subsequent decrease in both lipid and API content of the filtered product.

The lead formulation (DSPC, DSPG, IMM60 (weight ratio 5/4/1)) was prepared again at a 5 mL scale (reference lot E) to identify the limitations of the original manufacturing process. During manufacture, a single pass extrusion led to efficient size reduction to achieve vesicle size of −8 nm. However, the particle size increased upon processing through the diafiltration cartridge. This increase in size likely contributed to poor product filterability.

In-process determination of lipid and API content for lot E was monitored throughout manufacturing. Recovery of lipid and API was 95% after extrusion, but decreased to 55% after diafiltration and ultrafiltration to half the initial volume. The subsequent filtration step, using cellulose acetate membrane (0.2 m sterilizing grade filter), also resulted in extensive loss of product with only 18% of initial amount being recovered. Preliminary filtration tests with polyether sulphone (PES) instead of cellulose acetate (CA) membranes showed promising results that were further confirmed during process optimization.

The manufacturing process was optimized through Studies F, G, I, J and L. Particle size instability was first addressed in study F to optimize LUV particle size post-extrusion (cooling rate and solvent concentration). Additionally, to reduce particle size increase during ultrafiltration due to high solvent concentration, diafiltration (DF) was performed prior to ultrafiltration (UF). However, LUV size increase was still observed throughout the process, so study G further included immediate dilution of the LUV solution by extruding into either cold or hot buffer. Temperature of the buffer appears to have had no immediate effect on particle size, but dilution of the LUV solution prior to DF did improve particle size stability. Further stabilization of particle size was achieved by increasing the surface area of the cartridge from 20 cm² to 115 cm², thereby increasing the rate of diafiltration.

Study I confirmed a stable particle size upon extrusion into room temperature buffer (1:1); and improved lipid recovery by~9% when a cartridge with smaller pore size, 100 kD versus 500 kD, was used. A 50 mL batch (Lot J) using IMM60 was prepared using the modified procedures including a post-extrusion dilution (1:1) with RT buffer and diafiltration prior to ultrafiltration. The use of 100 KDa pore size cartridge for DF/UF was problematic and significantly increased the processing time. For this reason, the DF/UF was completed with a 115 cm², 500 kDa cartridge instead.

Production at ~7 mL scale was conducted in Study L to test a new lot of API with the new manufacturing process. No difficulties were faced with the API and particle size, lipid and API content were close to targets.

Aggregation of the liposomes was observed in early stability testing. Adding sucrose to the buffer and including an annealing step during manufacturing resulted in smaller increase in particle size and appeared more stable over time, compared to the original formulation prepared in buffer containing saline only. The results obtained with the DSPC/DSPG/CHOL/IMM60 formulation also indicate that lowering the DSPG content (compensated by increasing cholesterol at 20 mol %) may result in slight improvement to particle size stability after cold storage at 4° (13 days post DOM), in buffer containing sucrose (with or without annealing step).

The processes and resulting products described in this example and subsequent examples are contemplated as aspects of the invention.

### Example 2—Stability Testing

Two compositions as disclosed herein were formulated and the stability was tested. The first composition (Lot 1) was prepared according to Example 1 and formulated according to Table 4. Initial stability testing of the first composition included the testing of particle size of multi-lamellar vesicles (MLV), after addition of the liposomes into the buffer in a 1:1 dilution (Pass 1), after diafiltration (post DF), after ultrafiltration (post UF), and after clarifying filtration (post CF) shown in Table 5. The mean diameter (900 Z average) was 102. initially (T=0) shown in Table 5. The first composition was then stored in a buffer (250 mM Sucrose, 145 mM NaCl, 10 mM Na-Phosphate, pH 6.5) at 25° C., 5° C., and −20° C. Table shows the concentrations of the various components in the composition after 9 days and after 1 month. Table 7 shows the mean diameter and polydispersity index (PdI) at T=7, 14, 21, 28, 42, 57, 206, 20 when heat treated, 231, and 231 days when heat treated (Heated). The heat treatment includes heating the liposomes to 55° C. for 10 minutes after storage. Table 7 shows the formulation when stored at −20° C., 5° C., and 25° C. Exemplary tools for heating include heat blocks, hot plates, ovens, and water baths.

TABLE 4
DSPC/DSPG/IMM60

| | [Measured | | Molar |
|---|---|---|---|
| Lot 1 | Values] mg/mL | wt ratio | ratio |
| DSPC | 4.80 | 49.7% | 50 |
| DSPG | 3.85 | 39.8% | 40 |
| CHOL | 0.00 | 0.0% | 0 |
| IMM60 | 1.02 | 10.5% | 10 |
| Total Lipid | 9.67 | 100% | |

TABLE 5

| | Lipid | | | |
|---|---|---|---|---|
| | DSPC/DSPG/IMM60 (Lot 1) | | | |
| | Buffer composition | | | |
| | 250 mM Sucrose, 145 mM NaCl, 10 mM | | | |
| | Na-Phosphate, pH 6.5 | | | |
| Sample | 90° Z average | (PdI) | 12.8° Z Average | (PdI) |
| MLV | 194.4 | 0.216 | 372.8 | 0.189 |
| Pass 1 | 101.1 | 0.092 | 105.4 | 0.222 |
| (1:1 dilution) | | | | |
| Post DF | 101.3 | 0.123 | 99.48 | 0.25 |
| Post UF | 102 | 0.79 | 94 | 0.251 |
| Post CF | 102.6 | 0.084 | 91.48 | 0.301 |

| | |
|---|---|
| Monitoring Particle Size | Storage Condition |

| (Z average 90°) | 25° C. | 5° C. | −20° C. |
|---|---|---|---|
| DOM | 102.6 | NAPP | NAPP |
| 1 week | 100.1 | 104.6 | 123.7 |

TABLE 6

| | (T = 9 days) | | (T = 1 month) | | | | | |
|---|---|---|---|---|---|---|---|---|
| | Storage: 5° C. | | Storage: −20° C. | | Storage: 5° C. | | Storage: 25° C. | |
| | [Measured] | wt | [Measured] | wt | [Measured] | weight | [Measured] | wt |
| Lot 1 | mg/mL | ratio | mg/mL | ratio | mg/mL | % | mg/mL | ratio |
| DSPC | 4.80 | 50% | 4.92 | 49% | 4.86 | 48% | 4.69 | 48% |
| DSPG | 3.85 | 40% | 4.14 | 41% | 4.10 | 41% | 3.99 | 41% |
| IMM60 | 1.02 | 11% | 1.04 | 10% | 1.12 | 11% | 1.02 | 10% |
| Total Lipid | 9.67 | 100% | 10.10 | 100% | 10.08 | 100% | 9.70 | 100% |

TABLE 7
Particle Size (Storage for 7.5 months)

| | | Z ave 90°, | |
|---|---|---|---|
| | # Days | nm | PdI |
| DSPC:DSPG:IMM60 | DOM | 102.6 | 0.084 |
| storage 5 °C | 7 | 104.6 | 0.108 |
| | 14 | 106.5 | 0.139 |
| | 21 | 111 | 0.182 |
| | 28 | 128.4 | 0.338 |
| | 42 | 126.8 | 0.241 |
| | 57 | 144.1 | 0.486 |
| | 206 | 222 | 0.466 |
| Heated | 206 | 138 | 0.198 |
| | 231 | 181.5 | 0.395 |
| Heated | 231 | 142.6 | 0.238 |
| storage −20 °C | 7 | 123.7 | 0.106 |
| | 14 | 125.5 | 0.137 |
| | 21 | 161.5 | 0.173 |
| | 28 | 161.6 | 0.212 |
| | 42 | 120.1 | 0.083 |
| | 57 | 164 | 0.212 |
| | 206 | 188 | 0.241 |
| Heated | 206 | 103.2 | 0.096 |
| | 231 | 185.1 | 0.246 |
| Heated | 231 | 105.3 | 0.061 |
| storage 25 °C | 7 | 100.1 | 0.106 |
| | 14 | 99.62 | 0.145 |
| | 21 | 100.4 | 0.103 |
| | 28 | 100.7 | 0.113 |
| | 57 | 101.3 | 0.094 |

The second composition (Lot 2) was prepared according to Example 1 and formulated according to Table 8. Initial stability testing of the second composition included the testing of particle size after MLV, after addition of the liposomes into the buffer in a 1:1 dilution (Pass 1), after diafiltration (post DF), after ultrafiltration (post UF), and after clarifying filtration (post CF) shown in Table 9. The mean diameter (900 Z average) was 92.8 initially (T=0). The second composition was then stored in a buffer (250 mM Sucrose, 145 mM NaCl, 10 mM Na-Phosphate, pH 6.5) at 25° C., 5° C., and −20° C. Table 10 shows the concentrations of the various components in the composition after 8 days and after 1 month. Table 11 shows the mean diameter and polydispersity index (PdI) at T=7, 14, 21, 28, 42, 57, 206, and 20 heat treated. The heat treatment includes heating the liposomes to 55° C. for 10 minutes after storage. Table 11 shows the formulation when stored at −20° C., 5° C., and 25° C.

TABLE 8

| | [Measured | | Molar |
|---|---|---|---|
| Lot 2 | values] mg/mL | wt ratio | ratio |
| DSPC | 4.20 | 44.6% | 41 |
| DSPG | 3.18 | 33.8% | 31 |
| CHOL | 0.91 | 9.7% | 18 |
| IMM60 | 1.12 | 11.9% | 10 |
| Total Lipid | 9.41 | 100.0% | |

TABLE 9

| Lot | 2 | | | |
|---|---|---|---|---|
| Lipid | DSPC/DSPG/CHOLESTEROL/IMM60 | | | |
| | 250 mM Sucrose, 145 mM NaCl, | | | |
| | 10 mM Na-P, pH 6.5 | | | |
| | 90° Z | | 13° Z | |
| Sample | average | (PdI) | Average | (PdI) |
| MLV | 156.4 | 0.213 | 425.7 | 0.596 |
| Pass 1 (1:1 dilution) | 94.97 | 0.109 | 151.5 | 0.83 |
| Post DF | 92.36 | 0.115 | 117.9 | 0.102 |
| Post UF | 93.04 | 0.100 | 94.05 | 0.167 |
| Post CF | 92.8 | 0.127 | 77.44 | 0.363 |

| Monitoring | | | |
|---|---|---|---|
| Particle Size | Storage Condition | | |
| (Z average 90°) | 25 °C | 5 °C | −20 °C |
| DOM | 92.8 | NAPP | NAPP |
| 1 week | 93.2 | 103.1 | 156.9 |

TABLE 10

| | | | T = 1 month | | | | | |
|---|---|---|---|---|---|---|---|---|
| T = 8 | | | Storage: −20° C. | | Storage: 5° C. | | Storage: 25° C. | |
| | [Measured] | wt | [Measured] | wt | [Measured] | weight | [Measured] | wt |
| Lot 2 | mg/mL | ratio | mg/mL | ratio | mg/mL | % | mg/mL | ratio |
| DSPC | 4.2 | 45% | 4.16 | 44% | 4.03 | 43% | 3.99 | 43% |
| DSPG | 3.18 | 34% | 3.07 | 33% | 3.21 | 34% | 3.22 | 35% |
| CHOL | 0.91 | 10% | 1.05 | 11% | 0.98 | 11% | 0.92 | 10% |
| IMM60 | 1.12 | 12% | 1.09 | 12% | 1.13 | 12% | 1.05 | 11% |
| Total Lipid | 9.41 | 100% | 9.37 | 100% | 9.35 | 100% | 9.17 | 100% |

TABLE 11
Particle Size (7 months Storage)

| | | Z ave 90°, | |
|---|---|---|---|
| | # Days | nm | PdI |
| DSPC:DSPG:CHOL:IMM60 | DOM | 92.8 | 0.127 |
| storage 5 °C | 7 | 103.1 | 0.128 |
| | 14 | 101.9 | 0.071 |
| | 21 | 107.9 | 0.093 |
| | 28 | 110.2 | 0.104 |
| | 42 | 108.6 | 0.094 |
| | 57 | 109.6 | 0.14 |
| | 206 | 118.8 | 0.17 |
| Heated | 206 | 98.44 | 0.085 |
| storage −20 °C | 7 | 156.9 | 0.242 |
| | 14 | 142.3 | 0.185 |
| | 21 | 153.8 | 0.174 |
| | 28 | 164.9 | 0.265 |
| | 42 | 176.9 | 0.359 |
| | 57 | 166.8 | 0.317 |
| | 206 | 164.3 | 0.261 |
| Heated | 206 | 102.7 | 0.112 |
| storage 25 °C | 7 | 93.2 | 0.099 |
| | 14 | 90.04 | 0.11 |
| | 21 | 92.86 | 0.092 |
| | 28 | 93.65 | 0.11 |
| | 57 | 91.94 | 0.078 |

These data demonstrate that liposome preparations described herein are amenable to storage, especially at lower temperatures. The data further demonstrate that a heat treatment of the liposomes, especially stored liposomes, can reduce particle size nd PdI. Without intending to be limited to any particular theory, the heat treatment is contemplated to reduce liposome agglomeration.

Compositions comprising heat-treated liposomes are contemplated as an aspect of the invention. Methods of manufacture that include a heat treatment step are intended as an aspect of the invention. Methods of preparing or standarding liposomal compositions that include a heat treatment are intended as an aspect of the invention, especially for a liposomal composition that has been stored under freezing or sub-zero conditions or freeze-dried and reconstituted in an aqueous buffer. Methods of treatment that include a heat treatment step are contemplated as an aspect of the invention. In some embodiments, the heat treatment is for a time and at a temperature sufficient to reduce average particle size and/or reduce PdI of the composition. In exemplary embodiments, the heat treatment is at a temperature in the range of about 55° C. to about 65° C. In some embodiments, the heat treatment is for about 5 minutes to about 15 minutes, e.g., for about ten minutes.

Biology Studies of Liposomes that include IMM60 Example 3—Liposomal IMM60 Compared to Soluble IMM60

Liposomal IMM60 (DSPC:DSPG:IMM60) was compared to soluble IMM60 for its ability to generate an ovalbumin-specific immune response as well as its ability to mature dendritic cells and to induce accumulation of IFN-γ in the serum. The two IMM60 formulations (soluble or liposomal) were each co-injected with the ovalbumin antigen into C57BL/mice. When Liposomal IMM60 and soluble IMM60 were injected at 0.1 ng/mouse (approx. 4 ng/Kg), they induced similar levels of serum IFN-γ accumulation with no statistically significant different between them. However, liposomal IMM60 was significantly more potent at maturing splenic dendritic cells 18 hours after administration, and also resulted in significantly more CD8+ T cells that were specific for the ovalbumin peptide epitope SIINFEKL 7 days after administration.

The early stimulation capacity of these formulations was tested by measuring serum IFN-γ 18 hours after injection. High levels of IFN-γ in the serum at this time point is a well-established standard assay for iNKT cell activation, and it is used as a surrogate assay for the early activation of iNKT cell, which then cascades into amplified production of IFN-γ by NK cells. A second assay assessed the maturation level of splenic dendritic cells 18 hours after administration of the IMM60 formulations. Cognate interaction between iNKT cells and dendritic cells has been shown, in previous studies, to result in maturation of the dendritic cells in a CD40:CD40-ligand dependent manner. Two animals from each group were culled 18 hours after the injection of IMM60, and the splenic CD11c⁺ cells were examined for surface levels of CD86, a marker of maturation of dendritic cells. The remaining animals in each group were used, 7 days after the injection, to assess the number of T cell that specifically recognize ovalbumin-derived peptides (SIINFEKL). This was done by sampling the blood and staining blood lymphocytes with fluorescent tetrameric MHC class I monomer carrying the SIINFEKL peptide.

Mice are a chosen animal model of in vivo studies described herein since they are the lowest vertebrate group in the evolutionary tree for which suitable models of the immune responses in general, and iNKT biology in particular, are available, including mutant strains that lack either iNKT cells or CD1d molecules.

### Storage and Preparation Before Use:

Liposomal IMM60—Vials were thawed at room temperature (21° C.-22° C.). Vials were then heated to 55° C. for 10 minutes using a heated water bath, mixed by inversion times, cooled to room temperature, and diluted to the required concentration using tissue culture grade PBSA.

Soluble IMM60—Lyophilized material was resuspended in chloroform:methanol:water (10:10:3) to 10 mg/ml (vials of 100 μg in 10 μl) and swirled around to capture all the material, and then Vehicle solution (150 mM NaCl, 0.5% Tween20 in distilled water) was added quickly to a final concentration of 100 μg/ml (for 100 μg in 10 μl, added 990 μl of Vehicle). This composition is then heated to 80-90° C. for 5 minutes and sonicated with a waterbath sonicator for 5 minutes.

54 C57BL/6JOlaHsd female mice, 7-8 weeks old, were used in this study. Mice were purchased from Envigo and weighed 17-19 grams.
Maxisorb plates 9flat bottom half size wells (Nunc)
Capture antibody: Clone R4-6A2 (eBioscience)
Detection antibody: Clone XMG1.2-biotin (eBioscience)
Streptavidin-HRP (Thermo Fisher Scientific)
TMB substrate (Sigma)
Sulphuric acid 0.1N (Sigma)
3 Splenocyte processing
PBS, RPMI, Feotal Bovine Serum (FBS), Penicillin/Streptomycin (Pen/Strep), Glutanime, (all
Tissue Culture grade products from Gibco)
Red Blood Cell Lysis Solution (Qiagen)
All antibodies were purchased form eBioscience or Biolegend
CD11b-FITC, CD11b-PEcy7, CD105-BV421, CD45.2-APC, CD11c-APC-cy7, CD86-PE, PD-L1-PE, CD40-PEcy7, CD11c-APC, and MHC classII-APCcy7 (from Biolegend). B220-eFluor450 (from eBioscience). Live/Dead fixable stain (ThermoFisher scientific)
Fc blocking antibody anti CD16/CD32 (eBioscience)

### Preparation of Reagents:

Ovalbumin 400 μg/100 μl: 20 mgs of ovalbumin reconstituted in 5 ml with PBS (TC grade.
Liposomal IMM60 (stock 0.47 mg/ml):
   1st dilution: 4.7 ml PBS+10 μl of stock DSPC:DSPG:IMM60
   2nd dilution 10 ml of PBS+10 μl of 1st dilution
   3rd dilution 9 ml of PBS+1 ml of 2nd dilution
Soluble IMM60 (stock 100 μg/ml):
   1st dilution 5 ml PBS+5 μl stock
   2nd dilution 4.95 ml PBS+50 μl of 1st dilution

Mice were then warmed up for 20-30 minutes in cages place in a 37° C. heating chamber. For Group 1: 200 μl PBS was injected intravenously. For Group 2: 200 μl of a mix of 1:1 ovalbumin:PBS. For Group 3: 200 μl of a mix of 1:1 ovalbumin:soluble IMM60 (2^{nd} dilution). For Group 4: 200 μl of a mix of 1:1 ovalbumin:Liposomal IMM60 (2^{nd} dilution). For Group 5: 200 μl of a mix of 1:1 ovalbumin:Liposomal IMM60 (3^{rd} dilution).

Animals were injected intravenously in the lateral tail vein. Cages containing 3-mice were warmed in a heating chamber (37° C.) for 20-30 minutes. Individual mice were placed in a restrainer that allowed the tail to protrude out. Using 27G insulin syringe needles, mice were injected with 200 μl of the recorded dose over approximately 4 seconds. Group size is detailed in Table 12 below, and mice were housed in individually ventilated cages in groups of 4-6:

TABLE 12

| Group | Injected material | C57BL/6 | C57BL/6 |
|---|---|---|---|
| 1 | Naïve | 5 | 2 |
| 2 | ovalbumin+ vehicle | 6 | 2 |
| 3 | ovalbumin+ soluble | 11 | 2 |
| | IMM60 0.1 ng/mouse | | |
| 4 | ovalbumin+ liposomal | 11 | 2 |
| | IMM60 0.1 ng/mouse | | |
| 5 | ovalbumin+ liposomal | 11 | 2 |
| | IMM60 0.01 ng/mouse | | |
| | | 44 | 10 |
| | | | culled at 18 hrs |

18 hours after injection of the doses, blood samples were taken for analysis of serum and mice were euthanized and splenocytes were recovered for analysis of CD11c⁺ cells' maturation.

Blood sampling—Serum: Cages containing 3-mice were warmed in a 37° C. heating chamber for 20-30 minutes after which individual mice were placed in a restrainer leaving the tail exposed. Using a scalpel, a nick in the lateral tail vein was made and blood was collected into serum separation tubes. Blood flow was stopped by applying pressure and the mice returned to their normal housing cages. Blood was allowed to coagulate for 30 min. at room temperature and then serum was separated by centrifugation in a benchtop microcentrifuge for 2 minutes at 13000 g. Serum transferred to wells in a 9U bottomed well dish and frozen at −20° C. Thawed samples were later used without dilution in a standard Sandwich ELISA.

Blood Sampling—cells: Blood was collected from the lateral tail vein as above but instead of serum separation tubes, drops of blood (about 100 μl) were collected into a 1.5 ml eppendoff tube containing 200 μl of 10 mM EDTA/PBS solution and mixed by inverting times. Cells were kept on ice until a full cage of mice were bled. 1 ml of Red Blood Cell Lysis Solution (Qiagen) was added to the blood and mixed by inversion. Samples were kept on ice until they turned translucent. Tubes were then centrifuged on a microcentrifuge 4 KRPM for 4 minutes at room temperature. Supernatant was removed along with palettes leaving a buffy-coloured pellet. The cells in the pellet were washed with 200 μl of cold PBS and transferred into U-bottomed wells in a 96-well plate and pelleted for 2 minutes at 577 g (1800 rpm). Supernatant was removed and cells were treated with Fc Block (anti CD16/CD32 antibody). Pellet was resuspended in 10 μl of 1:10 dilution of tetrameric pMHC K^{b}/SIINFEKL, and the plate was covered in aluminum foil and incubated at 37° C. for 30 minutes. Cells were washed once in cold PBS and resuspended in a mixture of fluorescent antibodys and left on ice for 15 minutes, then washed twice in PBS+0.1% FBS before being diluted with 200 μl of the same solution and analysed using BD LSR Fortessa FACS machine.

Splenocytes: Mice were euthanised by a Schedule 1 procedure (cervical dislocation) and spleens were collected and kept in 1.5 ml RPMI on ice. Spleens were mashed and squeezed through a 70 μm sieve into a 50 ml Falcon tube using the flat end of a 1 ml syringe plunger, and the cells were pelleted by centrifugation at 1500 rpm for 5 minutes in an underbench centrifuge (484 g). Splenocytes were resuspended in 2 ml Red Cell Lysis Solution (Quiagen) and after 2 minutes at room temperature were diluted with 8 ml of full media (RPMI, 10% FBS, Pen/strep, Glut). Splenocytes were mixed and then pelleted by centrifugation (484 g) at room temperature. Splenocytes were then resuspended in 3mls of full media and counted. 2×1ealiquotes of the splenocytes were placed in a well of a plate of 9U bottom wells. Cell were pelleted by centrifugation 2 minutes at 1800 rpm (577 g) and the supernatant discarded. Each pellet was resuspended in a mixture of fluorescent antibodys and left on ice for 15 minutes then washed twice in PBS+0.1% FBS before being diluted with 200 μl of the same solution and analysed using BD LSR Fortessa FACS machine.

High protein binding (Maxisorb), 96-well flat bottom plates were coated with rat anti-mouse IFN-γ capture antibody clone R4-6A2 (5 μg/ml) diluted in sodium bicarbonate solution (0.05M pH=9.5) (Sigma). Plates were left overnight at 4° C. the night before, washed 3 times in PBS-0.005% Tween-20, and blocked with 2.5% FBS in PBS for 2 hours. After an additional three washes in PBS-0.005% Tween-20, serum samples were added to the plate as well as a duplicate set of serial dilution of standards (recombinant mouse IFN-γ (Peprotech)) ranging from 500 ng/ml down to 0.488 ng/ml and a duplicate blank (PBS). The plates were left overnight at 4° C.

Samples were then removed and washed times after which the detection antibody solution (biotinylated clone XMG1.2 (0.5 μg/ml)) was added and left in room temperature for 2 hours.

After a further 3 washes, a solution of streptavidin-HRP (×1000 dilution of Invitrogen stock solution) was added and left at room temperature for 1 hour. Plates were washed again in PBS-0.005% Tween-20 six times and twice in PBS. TMB (sigma) was added, covered in silver foil, and left at room temperature and inspected regularly. Colorimetric reaction was stopped by addition of an equal volume of 0.05 N sulfuric acid as soon at the 250 ng/ml standard reached saturation (about 10-15 minutes). Absorbance at 450 nm of the wells were analysed by SPECTROstar Nano plate reader. The SPECTROstar Nano plate reader's software converted the absorbance to ng/ml using the calibration curve generated by the standard dilutions.

Injection of either of the IMM60 formulations (co-injected with ovalbumin) resulted in high levels of IFN-γ accumulation in the serum of treated mice after 18 hours (FIG. 6). There was no statistically significant difference in the level of IFN-γ induction in these two groups. As has been discussed previously (Jukes J P et al., J. Eur. Immunol. 20146:1224-1234), non-glycosidic compounds can stimulate both human and mouse iNKT cells); the source of the IFN-γ is NK cells that had been transactivated into producing IFN-γ by iNKT cells which produce the same cytokine at an earlier stage following recognition of the iNKT cell agonist. In contrast, no induction of IFN-γ was observed in the animals that were untreated or treated with Vehicle (and ovalbumin) alone. Injection of liposomal IMM60 (co-injected with ovalbumin) produce intermediate amounts of serum IFN-γ.

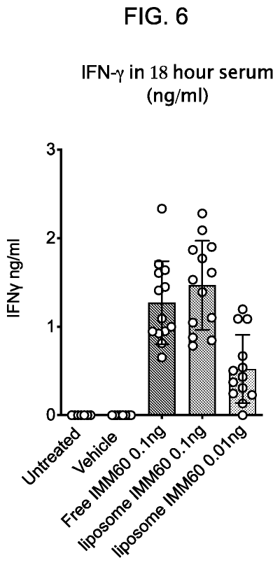

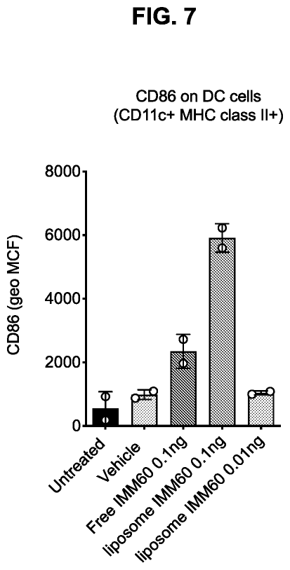

Maturation of CD11c⁺ dendritic cells in mice treated with liposomal IMM60 versus soluble IMM60: Maturation of dendritic cells was determined by measuring the levels of the costimulatory molecule CD8 on the surface of CD11c⁺ MHC class II+ cells. In this assay, a clear difference in the potency of IMM60 was observed when it was delivered as a soluble molecule compared to when it was delivered as a liposomal formulation (FIG. 7). Both treatments contained the same amount of IMM60 (0.1 ng/mouse), yet the liposomal formulation induced CD8levels dendritic cells that were 2.5 times higher. The experiment also shows that a 10-fold reduced dose of liposomal IMM60 did not mature dendritic cells beyond background levels at this time point. (FIG. 7.) This result contrasts with the observed stimulatory effect of systemic IFN-γ production using this same dose (FIG. 6).

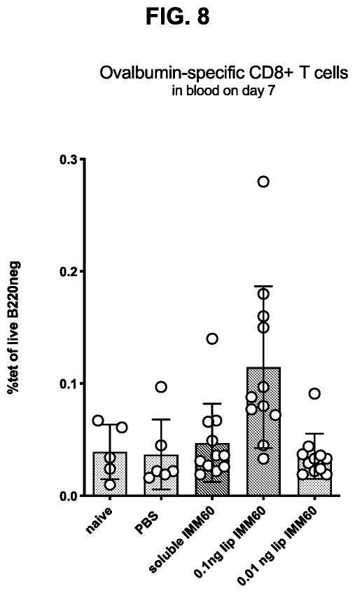

Seven days after injection of the mice, blood samples were taken, and blood cells were stained with fluorescent antibodies specific for lymphocyte markers as well as tetrameric pMHC: H-2K^{b} encompassing the ovalbumin peptide SIINFEKL. The percentage of T cells that bound the tetrameric pMHC is shown in FIG. 8; mice that received ovalbumin co-injected with liposomal IMM60 (0.1 ng/mouse) produced on average, significantly more antigen-specific T cells. This is in line with the data presented in FIG. 7 showing that CD11c+ MHC class II+ dendritic cells were highly mature in this group of mice and would have made much better antigen presenting cells compared to dendritic cells in the mice from any of the other groups.

Conclusions: The results of this experiment showed that the liposomal formulation of IMM60 enhanced the capacity of IMM60 to prime an antigen-specific immune response that would be vital for mounting an antitumour immune response. IFN-γ production had perhaps reached a maximum level in these conditions, but it was the dendritic cell maturation levels that correlated with the increased antigen specific T cell response, rather than the IFN-γ production. The results also showed that the maturation of dendritic cells, which was central to the translation of an early innate immune response into an adaptive immune response, was triggered efficiently by liposomal formulated IMM60.

### Example 4: Intravenous Injection of Six Different Liposomal Formulations of IMM60 (POPC/DDAB, DSPC/DSPG, and POPC/DC-Chol) into Mice

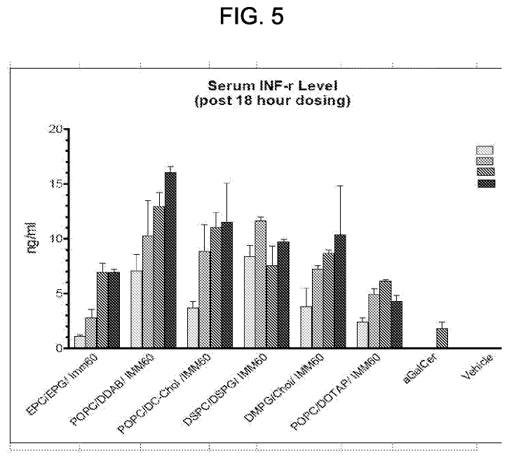

FIG. 5 shows results from an experiment to measure INF-gamma 18 hours after administration of six different prototype liposomal formulations of IMM60, compared to soluble αGalCer. This experiment showed that a DSPC/DSPG prototype liposome had the highest dynamic range (flat and elevated response across the doses) and the response at the lowest dose. The doses are (from left to right) 0.01, 0.1, 1, 5 ng/mL with a 100 uL injection. The positive control used for this experiment was soluble alpha-GalCer at 1.0 ng/mL. The data also showed that, for the second through fifth prototype liposomes, IFNγ measurements for the higher two doses are substantially elevated compared to soluble alpha-GalCer at 1.0 ng/mL.

### Example 5: Intravenous Injection of Three Different Liposomal Formulations of IMM60 (POPC/DDAB, DSPC/DSPG, and POPC/DC-Chol) into Mice

Three different IMM60 liposomal formulations (POPC/DDAB, DSPC/DSPG, and POPC/DC-Chol) were tested by IV injection in mice. The effect of the different liposomal preparations on IMM60's ability to induce serum levels of IFN-7 at 18 hours was determined, as well as the degree of iNKT cell-mediated maturation of splenic dendritic cells (DCs) and compensatory upregulation of PD-L1 (FIG. 1A-1C). CD1d knockout mice were used as the control, because CD1 knockout mice do not produce iNKT cells (due to lack of CD1 expression).

Three IMM60 liposome formulations, POPC/DDAB, DSPC/DSPG, and POPC/DC-Chol, were injected intravenously into either wild type C57BL/mice of CD1d KO mice, which do not have the presenting molecule DC1d nor invariant natural killer T cells. Eighteen hours later, serum levels of IFN-7 were measured and splenic DCs were tested for surface expression of CD86, CD40, and PD-L1.

The effect of IMM60 treatment on IFN-7 induction and DC maturation was shown to be target-dependent (CD1d), regardless of the liposome formulation, as there was no effect observed in the CD1d knockout mice (FIG. 1A-1C). A dose-dependent induction of serum IFN-7 at 18 hours was observed for all 3 liposome formulations. However, the POPC/DC-Chol formulation was significantly less potent at inducing IFN-7 than either POPC/DDAB or the DSPC/DSPG (liposomal IMM60) formulations. (See FIG. 1A.) Liposomal IMM60 resulted in both the broadest dose-response as well as the greatest increase in IFN-7 at the highest tested dose. All three liposome formulations produced comparable potent effects on both DC maturation, which were already maximal at the lowest tested dose. A compensatory increase of the expression of the immunosuppressive PD-L1 ligand on the surface of DCs was observed (FIG. 1C).

### Example 6: Intravenous Injection of Three Different Liposomal Formulations of IMM60 (POPC/DDAB, DSPC/DSPG, and POPC/DC-Chol) into Mice

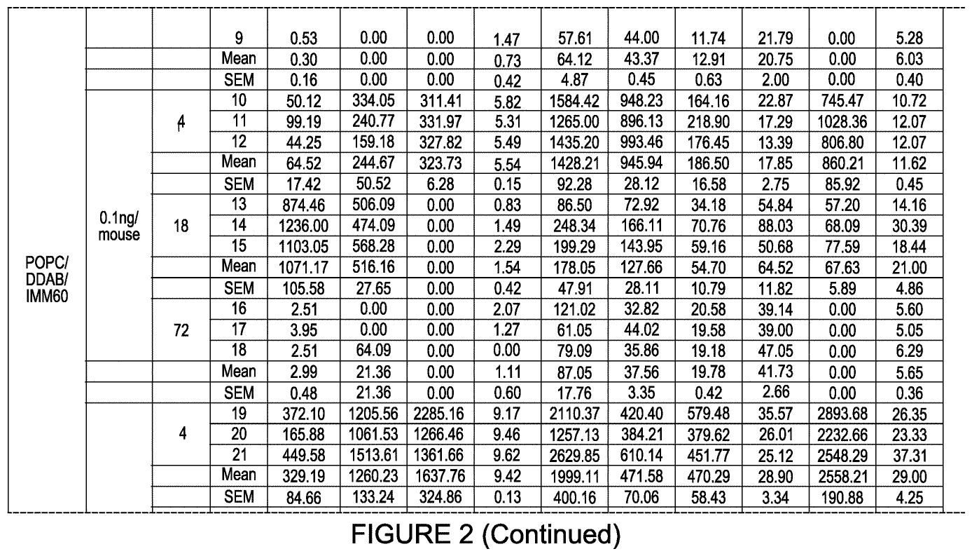

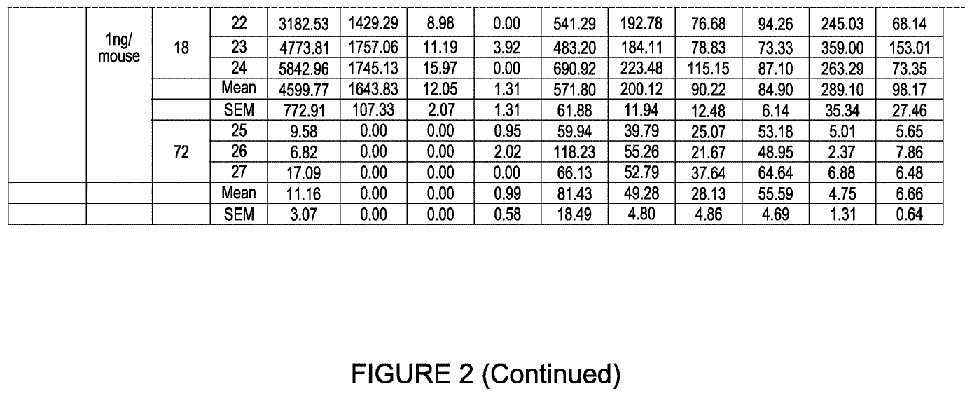

FIGS. 2-4 shows the results of an experiment in which 0.01 ng, 0.1 ng, and 1 ng of POPC/DDAB, DSPC/DSPG, or POPC/DC-Chol IMM60 formulated in liposomal compositions disclosed herein was intravenously injected into wildtype C57 BL/mice. The levels of IFNγ,IL-12p70, IL-2, IL6, and IL-4, were measured at 4 hours, 18 hours, and 72 hours post-injection. This experiment explored a lower dose range than Example 4 and added additional cytokine outcomes. These liposomal measurements were compared to measurements of the same markers when solubilized IMM60 (soluble IMM60, not formulated in liposomes) was injected as a positive comparator control and with a vehicle negative control.

### Example 7: Stability of Liposomal IMM60 In Vivo Following Intravenous Injection of Mice

### Synopsis

To estimate the biological half-life of IMM60 following an intravenous injection of liposomal IMM60, levels of IMM60 in the plasma were measured in two strains of mice, injected with two doses (0.5 and 50 μg/Kg), 10 minutes or 180 minutes after injection of Liposomal IMM60. The two strains of mice that were compared were CD1 mice (not to be confused with CD1d-KO mice) and C57BL/mice. The half-life, volume of distribution, clearance, AUC, and Cmax were calculated using the one-compartmental model with first-order kinetics. The group mean plasma concentration indicated a dose proportional response between the low (0.5 g/kg) and high (50 g/kg) doses in both mouse strains. Comparable values were obtained in both strains of mice for half-life, volume of distribution, and clearance. Values for soluble IMM60 injected mice were also obtained in which a longer half-life and larger volume of distribution were observed for IMM60.

The estimated half-life for the C57BL/and CD1 mice, 50 mg/kg dose, were 47.3 and 52.4 minutes respectively. Due to the exposures being lower than the limit of quantification for the low dose animals at 180 minutes, half-life could not be estimated for the 0.5 mg/kg dose for either strain of mice.

Lipid formulation: DSPG:DSPC:IMM60 (3.44:4.37:1 mg/ml) in a 250 mM sucrose, 145 mM NaCl, 10 mM sodium phosphate, pH 6.5 buffer. Before use, Vials of liposomal IMM60 were thawed at room temperature (21° C.-22° C.), then heated to 55° C. for 10 minutes using a heated water bath. Vials were mixed by inversion six times, cooled to room temperature, and diluted to either 100 ng/ml or 10,000 ng/ml using injection grade saline (0.9% NaCl).

Soluble IMM60: Lyophilized material was resuspended in Chloroform:methanol:water (10:10:3) to 10 mg/ml (vials of 100 μg in 10 μl) swirled around to capture all the material and then Vehicle solution (150 mM NaCl, 0.5% Tween20 in distilled water) added quickly to a final concentration of 100 μg/ml (for 100 μg in 10 μl, added 990 μl of Vehicle), then then heated to 80-90° C. for 5 minutes, and sonicated with a waterbath sonicator for 5 minutes. Samples were diluted to 10,000 ng/ml using injection grade saline.

Mice were warmed up for 20-30 minutes in cages place in a 37° C. heating chamber. Mice within each group were injected intravenously, using 27G insulin syringe needles, with 100 μl (C57BL/mice) or 150 μl (CD-1 mice) of the recorded dose over approximately 4 seconds.

Plasma samples were analyzed by LC/MS/MS at Frontage Laboratories. The assay used to measure IMM60 in the plasma after administration of liposomal IMM60 injection did not distinguish liposome-bound IMM60 from the soluble (unbound) form of IMM60. The method uses alpha GalCer (analog to IMM60) as an internal standard and the mass spectroctoscopic ionization is electrospray in the positive ionization mode. The range of calibration standards is between 1 ng/mL to 1000 ng/mL IMM60 in mouse plasma.

Plasma concentrations were measured 10 and 180 minutes after IV administration. The half-life, clearance, and AUC_{(0-∞)} were calculated using the one-compartmental model with first-order kinetics since only two time points were samples along the PK profile. Using these assumptions allow for the calculation of PK parameters. Other assumption is that the T_{10 minutes} is substituted for T₀ and C₁₀ minutes is substituted for C₀ and Cₘₐₓ to solve for PK parameters of liposomal IMM60.

$$Calculations of Half life of IMM 60 {t}_{1 / 2} = 0.693 / {k}_{e} where {k}_{e} = ( ln {C}_{10 min} - ln {C}_{180 min )} / ( {180}_{min} - {10}_{min} ) . \qquad ( 1 )$$

$${V}_{d} = Dose / {C}_{10 min} where dose is expressed as is or dose / body weight \qquad ( 2 )$$

$$Cl = {k}_{e} \times {V}_{d} = ( ln {C}_{10 min} - ln {C}_{180 min )} / ( {180}_{min} - {10}_{min} ) \times {V}_{d} \qquad ( 3 )$$

$$Calculation of AUC of IMM 60 {AUC}_{( 0 \to ∞ )} = {C}_{10 min} / {k}_{e} \qquad ( 4 )$$

Statistical Calculations: the mean, standard deviation (SD), standard error of the mean (SE), p-value from the t-test (equal or unequal variances) was calculated using Microsoft Excel 201 (Microsoft Corporation). Equal or unequal variances were chosen based on use of the F-test function. All t-test comparisons were performed with the 1000 ng IMM60 in liposome form as the control. Half-life T(1/2) in Mouse of IMM60: The half-life for mice of IMM60 is calculated using Equation 1 using the one-compartment model with 1^{st} order kinetics.

TABLE 13
Half-life in Mouse of IMM60

| half | 1000 ng | 1000 ng | 1500 ng | 1500 ng |
|---|---|---|---|---|
| life | IMM60 | IMM60 | IMM60 | IMM60 |
| (min) | in liposome | soluble | in liposome | soluble |
| | C57BL/6 | C57BL/6 | CD1 | CD1 |
| | 49.00 | 90.93 | 63.56 | 159.41 |
| | 50.97 | 98.13 | 62.36 | 118.64 |
| | 42.94 | | 37.80 | |
| | 46.25 | | 46.02 | |
| Average | 47.29 | 94.53 | 52.43 | 139.03 |
| STD | 3.49 | 5.09 | 12.62 | 28.82 |
| SE | 1.74 | 3.60 | 6.31 | 20.38 |
| N | 4 | 2 | 4 | 2 |
| p-value | | 0.00 | 0.46 | 0.14 |

Volume of distribution: The volume of distribution in mice of IMM60 is calculated using Equation 2 using the one-compartment model with 1^{st} order kinetics.

TABLE 14
Volume of distribution V(D) in Mouse of IMM60

| | 1000 ng | 1000 ng | 10 ng | 1500 ng | 1500 ng | 15 ng |
|---|---|---|---|---|---|---|
| | IMM60 in | IMM60 | IMM60 in | IMM60 in | IMM60 | IMM60 in |
| | liposome | soluble | liposome | liposome | soluble | liposome |
| | C57BL/6 | C57BL/6 | C57BL/6 | CD1 | CD1 | CD1 |
| Vd (mL) | 8.77 | 10.91 | 3.41 | 10.00 | 24.04 | 10.14 |
| | 8.85 | 11.40 | 7.41 | 4.49 | 14.29 | 5.66 |
| | 6.45 | | 4.88 | 9.93 | | |
| | 6.58 | | | 11.90 | | |
| Average | 7.66 | 11.15 | 5.23 | 9.08 | 19.16 | 7.90 |
| STD | 1.33 | 0.35 | 2.02 | 3.19 | 6.90 | 3.16 |
| SE | 0.66 | 0.25 | 1.17 | 1.60 | 4.88 | 2.24 |
| N | 4 | 2 | 3 | 4 | 2 | 2 |
| p-value | | 0.01 | 0.16 | 0.44 | 0.25 | 0.93 |

Clearance of IMM60: The clearance in mice of IMM60 is calculated using Equation 3 using the one-compartment model with 1^{st} order kinetics.

TABLE 15
Clearance in Mouse of IMM60

| | 1000 ng | 1000 ng | 1500 ng | 1500 ng |
|---|---|---|---|---|
| Clearance | IMM60 | IMM60 | IMM60 | IMM60 |
| (mL/min) | in liposome | soluble | in liposome | soluble |
| | C57BL/6 | C57BL/6 | CD1 | CD1 |
| | 0.12 | 0.08 | 0.11 | 0.10 |
| | 0.12 | 0.08 | 0.05 | 0.08 |
| | 0.10 | | 0.18 | |
| | 0.10 | | 0.18 | |
| Average | 0.11 | 0.08 | 0.13 | 0.09 |
| STD | 0.01 | 0.00 | 0.06 | 0.01 |
| SE | 0.01 | 0.00 | 0.03 | 0.01 |
| N | 4 | 2 | 4 | 2 |
| p-value | | 0.03 | 0.61 | 0.19 |

Exposure (AUC_{(0-∞)}) of IMM60: The exposure (AUC_{(0-∞)}) in mice of IMM60 is calculated using Equation 4 using the one-compartment model with 1^{st} order kinetics.

TABLE 16
Exposure (AUC(0-∞)) in Mouse of IMM60

| | 1000 ng | 1000 ng | 1500 ng | 1500 ng |
|---|---|---|---|---|
| AUC(0-∞)) | IMM60 | IMM60 | IMM60 | IMM60 |
| (ng/ml/min) | in liposome | soluble | in liposome | soluble |
| | C57BL/6 | C57BL/6 | CD1 | CD1 |
| | 8,061 | 12,032 | 13,757 | 14,353 |
| | 8,311 | 12,418 | 30,056 | 17,976 |
| | 9,603 | | 8,236 | |
| | 10,144 | | 8,367 | |
| Average | 9,030 | 12,225 | 15,104 | 16,165 |
| STD | 1,004 | 273 | 10,294 | 2,562 |
| SE | 502 | 193 | 5,147 | 1,812 |
| N | 4 | 2 | 4 | 2 |
| p-value | | 0.01 | 0.32 | 0.01 |

Cₘₐₓ of IMM60: The Cₘₐₓ in mice of IMM60 is calculated using the T_{10Min} time point concentration.

TABLE 17
Cmax in Mouse of IMM60

| | 1000 ng | 1000 ng | 10 ng | 1500 ng | 1500 ng | 15 ng |
|---|---|---|---|---|---|---|
| | IMM60 in | IMM60 | IMM60 in | IMM60 in | IMM60 | IMM60 in |
| | liposome | soluble | liposome | liposome | soluble | liposome |
| | C57BL/6 | C57BL/6 | C57BL/6 | CD1 | CD1 | CD1 |
| Cₘₐₓ (ng/mL) | 114.0 | 91.7 | 2.9 | 150.0 | 62.4 | 1.5 |
| | 113.0 | 87.7 | 1.4 | 334.0 | 105.0 | 2.7 |
| | 155.0 | | 2.1 | 151.0 | | |
| | 152.0 | | | 126.0 | | |
| Average | 133.5 | 89.7 | 2.1 | 190.3 | 83.7 | 2.1 |
| STD | 23.1 | 2.8 | 0.8 | 96.5 | 30.1 | 0.8 |
| SE | 11.6 | 2.0 | 0.5 | 48.3 | 21.3 | 0.6 |
| N | 4 | 2 | 3 | 4 | 2 | 2 |
| p-value | | 0.07 | 0.00 | 0.33 | 0.08 | 0.00 |

Pharmacokinetic Parameters for the High Dose (50 g/kg) Liposomal and Soluble IMM60 Injection in Mice.

The PK summary in mice of IMM60 is calculated using the equations above using the one-compartment model with 1^{st} order kinetics.

TABLE 18
Pharmacokinetic parameters for High Dose Liposomal
IMM60 injection in Mice

| | 1000 ng | 1000 ng | 1500 ng | 1500 ng |
|---|---|---|---|---|
| | IMM60 in | IMM60 | IMM60 in | IMM60 |
| | liposome | soluble | liposome | soluble |
| Dose (ng) | 1000 | 1000 | 1500 | 1500 |
| Weight (g) | 20 | 20 | 30 | 30 |
| Dosage | 50 | 50 | 50 | 50 |
| Level | | | | |
| (μg/kg | | | | |
| body | | | | |
| weight) | | | | |

PK Parameters, Mean ± SE

| | | | | |
|---|---|---|---|---|
| Cmax | 134 ± 12 | 90 ± 2 | 190 ± 48 | 84 ± 21 |
| (ng/ml) | | | | |
| t_{1/2} (min) | 47.3 ± 1.7 | 94.5 ± 3.6 | 52.4 ± 6.3 | 139.0 ± 20.4 |
| V_{d} | 383 ± 66 | 558 ± 12 | 303 ± 53 | 639 ± 163 |
| (mL/kg) | | | | |
| Cl | 0.11 ± 0.01 | 0.08 ± 0.00 | 0.13 ± 0.03 | 0.09 ± 0.01 |
| (ml/min) | | | | |
| Cl | 5.59 ± 0.31 | 4.09 ± 0.06 | 4.34 ± 1.05 | 3.13 ± 0.35 |
| (ml/min/ | | | | |
| kg) | | | | |
| AUC(0-∞) | 9,030 ± 502 | 12,225 ± 193 | 15,104 ± 5,147 | 16,165 ± 1,812 |
| (ng/ml/ | | | | |
| min) | | | | |

### Analysis

All comparisons are made assuming the high dose C57BL/strain as the reference for comparison.

Half-Life: The half life of the liposomal IMM60 formulation in both the C57BL/and CD1 strains were considered equivalent with T₁,2between 47 to 52 minutes for the high dose. The low dose could not be calculated due to the 3-hour plasma samples being less than the assay LOQ. The half life of the soluble formulation appears longer at the high dose with 95 to 139 minutes.

Volume of Distribution: The volume of distribution of the liposomal IMM60 formulation in both the C57BL/and CD1 strains were considered equivalent with V_{d}between 303 to 383 to ml/kg for the high dose. The low dose for each strain was also considered equivalent with V_{d} between 262 to 263 ml/kg. The volume of distribution of the soluble formulation appears larger with V_{d} between 558 to 639 ml/kg.

Clearance: The clearance of the liposomal IMM60 formulation in both the C57BL/and CD1 strains were considered equivalent with the clearance between 4.3-5.0 ml/kg/min for the high dose. The low dose could not be calculated due to the 3 hour plasma samples being less than the assay LOQ. The clearance of the soluble formulation appears lower with the values between 3.1 to 4.1 ml/kg/min.

Exposure: The exposure of the liposomal IMM60 formulation in both the C57BL/and CD1 strains were considered equivalent with AUC_{(0-∞)} between 9,030 to 15,104 ng/mL/min for the high dose. The low dose could not be calculated due to the 3-hour plasma samples being less than the assay LOQ. The exposure of the soluble formulation appears larger at the high dose with AUC_{(0-∞)} between 12,225 to 16,165 ng/ml/min.

Cₘₐₓ: The Cₘₐₓ of the liposomal IMM60 formulation in both the C57BL/and CD1 strains were considered equivalent with concentration between 134 to 190 to ng/ml for the high dose. The low dose for each strain was also considered equivalent, when scaled for the dose proportionality using the IMM60 dose in μg/kg for the low and high dose animals (low dose is 100-fold lower than the high dose). The Cₘₐₓ for the low dose was approximately 2.1 ng/mL for both mice strains. The Cₘₐₓ of the soluble formulation (high dose) appears lower with concentration between 84 to 90 ng/mL. The greater Cₘₐₓ for the liposomal formulation is considered favorable and an indicia of improved therapeutic efficacy for the liposomal formulation. In some embodiments the invention includes liposomal formulations of the API that, when administered to C57BL/or CD1 mice, provide a Cₘₐₓ at least 20%, or at least 30%, or at least 40%, or at least 50%, or at least 75%, or at least 100% greater than the Cₘₐₓ observed with an equivalent size dose of soluble API.

There were no pharmacokinetic differences detected between the C57BL/and CD1 mouse strains. There was no PK difference detected between either the high dose or low dose for volume of distribution and dose proportional Cₘₐₓ. The half life, clearance and exposure could not be calculated due to the 3-hour plasma samples being less than the assay LOQ. There were pharmacokinetic differences in T_{1/2}, Vd, clearance, exposure, and Cₘₐₓ detected between the liposomal and soluble formulation at the high dose.

The lower Volume of Distribution (V_{d}) of the liposomal formulation is favorable, indicating that the IMM60 of the liposomal formulation was 32% to 53% more concentrated in the plasma right after the injection compared to soluble formulation. The lower Vd indicates less of the IMM60 partitions outside of the plasma (blood) compartment. Since more IMM60 is in blood immediately after injection, more IMM60 can extravasate across tumor vasculature to penetrate tumors. Due to having a lower Vd, the liposomes were more efficacious and less toxic than the soluble IMM60. In some embodiments the invention includes liposomal formulations of the API that, when administered to C57BL/or CD1 mice, provide a Vd (measured in (mL/kg) that is at least 20%, or at least 30%, or at least 40%, or at least 50% less than the V_{d} observed with an equivalent size dose of soluble API.

### Example 8: Mouse Melanoma Injections of Lipsomal IMM60

To evaluate anti-cancer therapeutic properties of liposomal IMM60, the potential of liposomal IMM60 to prevent lung metastatic lesions in a mouse melanoma model was tested in both a prophylactic and therapeutic setting. Three doses of liposomal IMM60 were injected IV either 3 days before (prophylactic) or 3 days after (therapeutic) tumor cell injection. Mice were injected IV with B16-F10 melanoma cells and 15 days later metastatic nodules in the lung were counted.

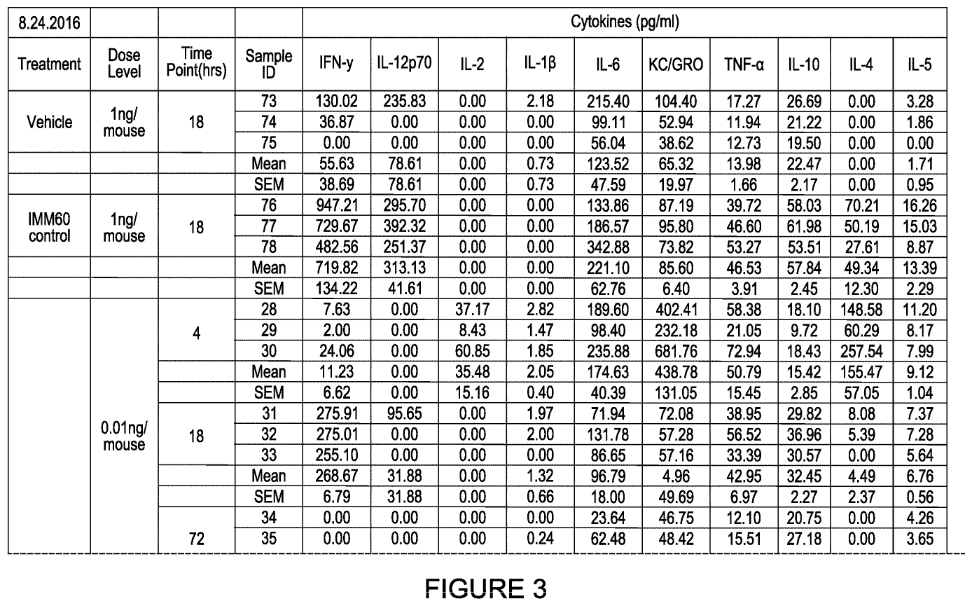

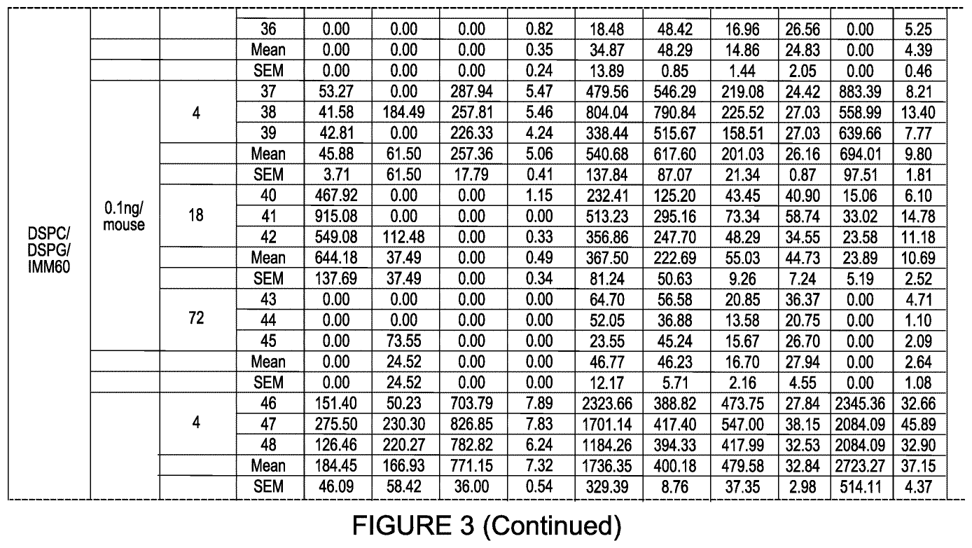

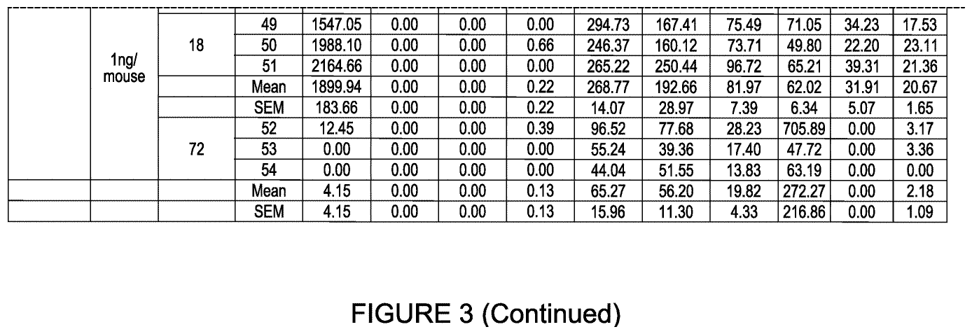

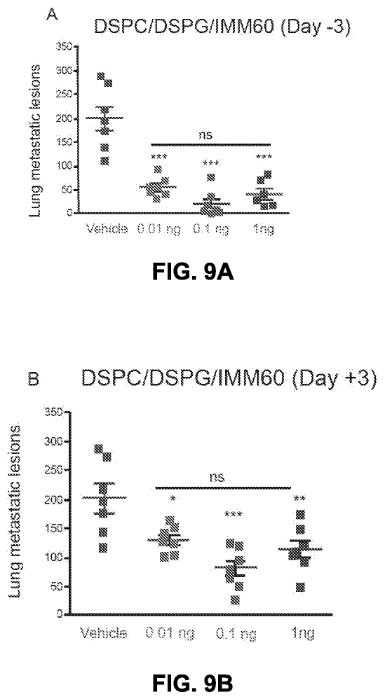

Prophylactic treatment was more effective than therapeutic treatment at preventing tumour formation in the lungs; nevertheless, with both prophylactic and therapeutic treatments, there was significant tumor reduction compared with mock-treated mice even at the lowest liposomal IMM60 dose (0.01 ng/mouse) (FIGS. 9A and 9B). In fact, maximal effect was found at 0.01 ng liposomal IMM60/mouse, with no significant additional benefit found at higher liposomal IMM60 doses. Treatment of mice with 1 of 3 doses (0.01, 0.1, or 1 ng per mouse) of liposomal IMM60 (DSPC/DSPG-IMM60) either 3 days before the tumour administration (prophylactic setting; FIG. 9A) or 3 days after the tumours were administered (therapeutic setting; FIG. 9B) resulted in the reduction of the number of metastatic nodules in the lungs of the mice.

### Example 9: Combination Therapy with Anti-PD-1 Blocking Antigen—B1Tumor model

Treatment with soluble IMM60 can upregulate the levels of PD-1 on iNKT cells. The same treatment also upregulates expression of PD-L1, the ligand for PD-1, on a variety of immune cells. (See, e.g., FIGS. 1C and 1F [liposomal IMM60]). The possibility that a combination treatment, with both liposomal IMM60 and anti-PD-1 blocking antibodies, could be used to enhance the inhibition of tumour growth, was examined.

A nonresponsive B16-F10 mouse melanoma SC model (“cold tumour model”) was utilized to assess the combination treatment in a therapeutic setting where the treatments were administered on Days 3, 6, and 9 after subcutaneous tumour implantations. Two dose levels of liposomal IMM60 were tested together with the immune checkpoint inhibitor anti-PD-1 antibody (10 mg/kg), and compared with either liposomal IMM60 alone, anti-PD-1 antibody alone, or an isotype control antibody.

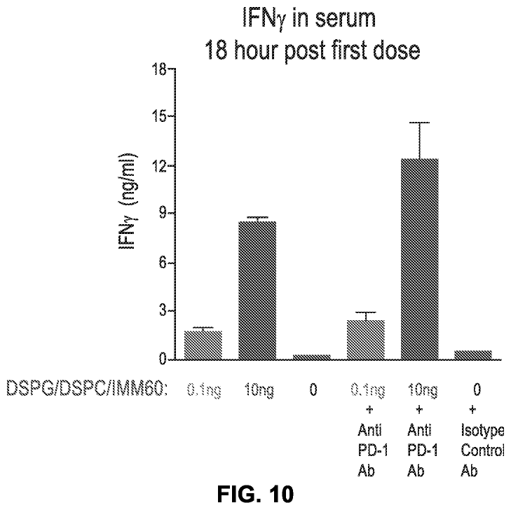

Eighteen hours after liposomal IMM60 treatment, blood samples were tested for IFN-γ. As shown in FIG. 10, the liposomal IMM60 activity was dose-proportional, with the higher dose (10 ng/mouse) producing more IFN-γ than the lower dose (0.1 ng/mouse). The treatment with the anti-PD-1 antibody alone had no effect on serum IFN-γ. Importantly, the combination of the anti-PD-1 antibody with the 10-ng dose/mouse of liposomal IMM60 produced higher levels of IFN-γ compared with liposomal IMM60 treatment alone.

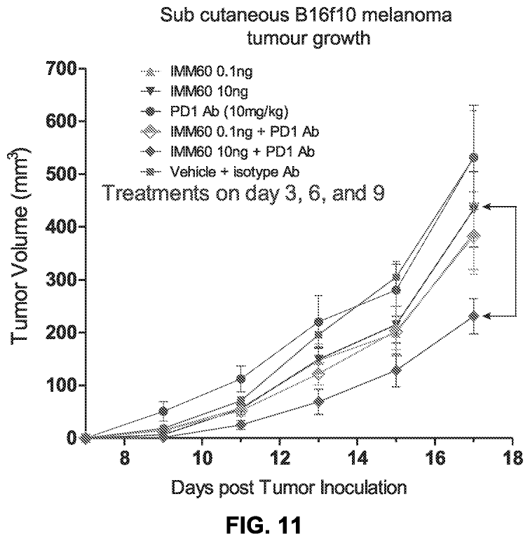

As expected, anti-PD-1 treatment alone had no effect on B16-F10 tumour growth, and the single-agent, liposomal IMM60, at either a low (0.1 ng/mouse) or high (10 ng/mouse) dose, had a modest antitumour effect. However, the combination of anti-PD-1 plus high dose liposomal IMM60 (10 ng/mouse) produced a significant decrease in tumour growth (as indicated by the arrows in FIG. 11).

### Example 10: Combination Therapy with Anti-PD-1 Blocking Antigen—CT-2Tumor Model

### Overview

The effects of IMM60 liposomes as a stand-alone treatment or in combination with anti-mouse PD-1 on tumor growth and survival in the CT-2tumor model in BALB/c mice was assessed, and IFN-γ levels in the serum of experimental mice were measured 18 hours after the first treatment.

Tumor volumes in the mice were measured over 14 days. In the fold-change over time, the interaction showed a trend towards statistical significance and the effect of treatment approached a trend towards significance and the post-hoc analysis detected a significant difference between the IMM60/anti-PD1 and vehicle control groups on Days 11 and 14.

Survival curves were not significantly different due to treatment.

Serum levels of IFN-7 were highly elevated in the IMM60 and IMM60/anti-PD1 treatment groups relative to the anti-PD1 group or vehicle controls. IMM60 in combination with anti-PD1 resulted in IFN-7 levels that were approximately two times higher than treatment with IMM60 alone.

### Experimental Details and Results

Body Weights and Tumor Measurements: Body weights of animals were taken on Days −1, 0, 2, 4, 7, 9, 11, 14, land 18. Tumors were measured on study Days 0, 2, 4, 7, 9, 11, 14, land 18. Tumors were measured in two dimensions using calipers. Tumor volumes were calculated using the formula: Volume (mm³)=(length×width²)/2.

Sixty-four mice received a subcutaneous inoculation of 0.3×10CT-2cells. Grouping and treatments for 32 mice were initiated when the mean tumor volume reached approximately 100 mm³ on Day 0, ten days after CT-2cells were inoculated. The four treatment groups received vehicle, IMM60, anti-PD1, or IMM60 in combination with anti-PD1 as outlined in the following Table.

### Experimental Design of the Main Study

| | | | | | Treatment | # Mice/ | |
|---|---|---|---|---|---|---|---|
| Group # | Treatment | Dose | | ROA | Schedule | Group | Study End Point |
| 1 | IMM60 liposome | 10 | ng/mouse | IV | Day 0 | 8 | Pseudo-survival |
| 2 | Anti-mouse PD-1 | 10 | mg/kg | IP | Days 0, 2, 5 | 8 | to 2,000 mm³ |
| 3 | IMM60 liposome | 10 | ng/mouse | IV | Day 0 | 8 | Levels of serum |
| | Anti-mouse PD-1 | 10 | mg/kg | IP | Days 0, 2, 5 | | IFN-γ 18 hours |
| 4 | Vehicle | NA | | IV | Day 0 | 8 | after administration |
| | Isotype control | 10 | mg/kg | IP | Days 0, 2, 5 | | of exosomes |

IV-intravenous
IP-intraperitoneal

Serum for IFN-γ Levels: Mice from each group were bled via the retro-orbital sinus under isoflurane anesthesia on Day 1, eighteen hours after the first treatment. Serum was separated from whole blood by clotting for 30′ at room temperature and centrifuging for 15′ at 1000×g, frozen at −80° C. until analysis. IFN-γ levels were determined using Meso Scale Discovery (MSD), according to the manufacturer's protocol.

Body weights of the animals in different groups closely tracked each other over the study period.

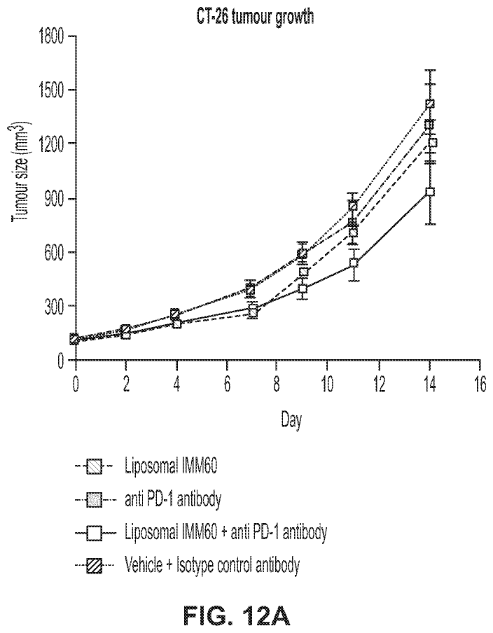

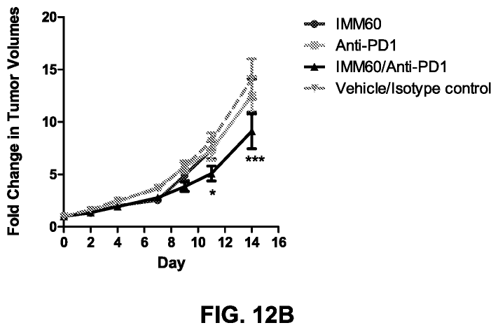

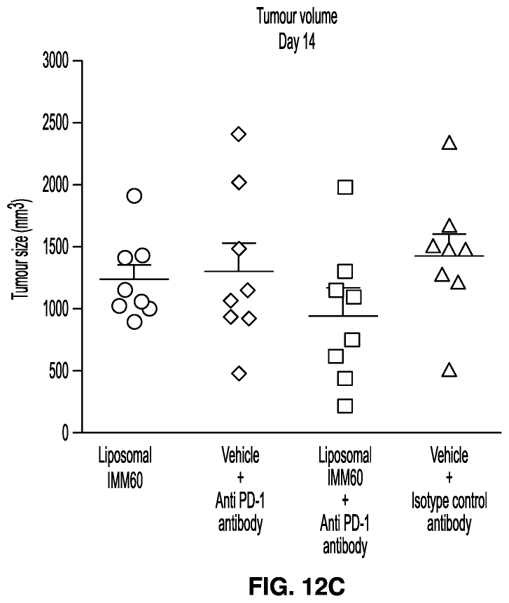

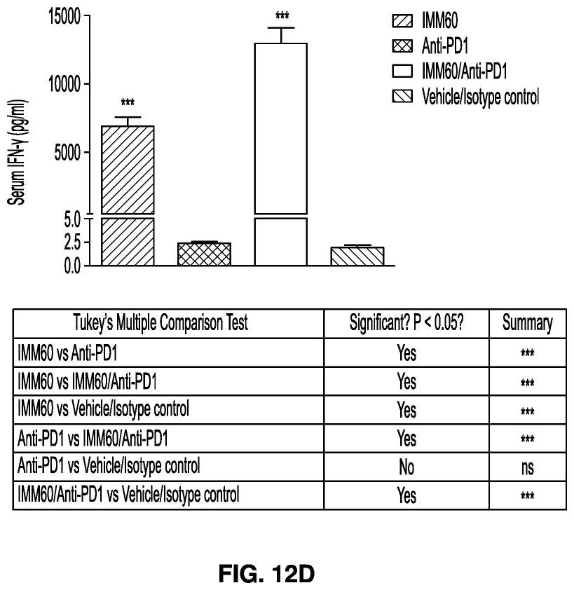

Tumor volume measurement data is depicted in FIG. 12A. When these data were analyzed by a two-way repeated measures ANOVA, the interaction showed a trend towards statistical significance (P=0.0698), and the effect of treatment approached a trend towards significance (P=0.1142). Bonferroni's post-hoc analysis is shown in FIG. 12B and detected a significant difference between the IMM60/anti-PD1 and vehicle control groups on Days 11 and 14.

Tumor volumes for individual animals are shown in FIG. 12C. The one-way ANOVA of these data did not show a significant difference due to treatment (P=0.3423).

IFN-γ levels measured at 18 hours are plotted in FIG. 12D. A significant effect of treatment was detected in the one-way ANOVA (P<0.0001). The levels of IFN-γ were highly elevated in the IMM60 and IMM60/anti-PD1 treatment groups relative to the anti-PD1 group or vehicle controls. The statistical differences in treatment groups versus the vehicle controls are show in the graph. Tukey's post-hoc analysis comparing all groups is shown below the graph. IMM60 in combination with anti-PD1 resulted in IFN-γ levels that were approximately two times higher than treatment with IMM60 alone.

These data from Examples 9 and 10 demonstrate that in circumstances in which the IFN-γ response to iNKT cell stimulation was limited, the addition of a complimentary immune checkpoint inhibition treatment increased the response to tumors that do not respond to either treatment alone.

### Example 11—IMM60 Induces Expression of PD-L1 on Melanoma Cells Co Cultured with Mouse Splenocytes

### Overview

Using an in vitro mouse cell model, we measured the effect of IMM60 on levels of PD-L1 expressed on the surface of tumour cells that are co-cultured with splenocytes. More specifically, we examined the PD-L1 expression on mouse melanoma tumor cell line B16f10 co-cultured with splenocytes (as a source of iNKT cells and antigen-presenting cells (APC) such as CD1⁺ B cells and CD11c⁺ dendritic cells that are present in the ex vivo splenocytes at about 1% of live cells).

Splenocytes from wild type mice were treated with either IMM60, IMM47 or vehicle and with anti-PD-1 antibody or an isotype control antibody. After 48 hours, PD-L1 levels on the surface of B16f10 cells were determined by antibody labeling and flow cytometry.

The addition of IMM60 resulted in up-regulation of surface PD-L1 on mouse melanoma cell line, B16f10, whereas addition of a less potent iNKT agonist, IMM47, did not upregulate surface PD-L1.

We also assessed the effect of check point inhibition on this up-regulation of PD-L1 expression. The co-administration of an anti-PD-1 antibody together with the IMM60, increased the level of PD-L1 on the melanoma cells; this enhancement was not observed on T cells.

### Materials and Methods

### Reagents and Preparation of Materials:

IMM60 and IMM47 are stored lyophilized at −20° C.

Soluble IMM60 or IMM47: Lyophilized material was resuspended in Chloroform:methanol:water (10:10:3) to 10 mg/ml (vials of 100 μg in 10 μl), swirled around to capture all the material, and then Vehicle solution (150 mM NaCl, 0.5% Tween20 in distilled water) added quickly to a final concentration of 100 μg/ml (for 100 μg in 10 μl, added 990 μl of Vehicle). This solution is then heated to 80° C. for 5 minutes and sonicated using a water-bath sonicator for 5 minutes. After reconstitution in glass vials, both IMM60 and IMM47 are stored at 4° C., and before each use they are sonicated in a water bath sonicator for 5 minutes.

The antibodies used were an anti-PD-1 antibody (rat anti-mouse PD-1) clone RMP1-14 (IgG2a, k) from BioXCell InVivoPlus and an Isotype control antibody (rat IgG2a,k) clone 2A3 from BioXCell InVivoPlus. Antibodies were diluted to the required concentration using complete medium (RPMI, 10% FBS, Pen/Strep and Glutamine) before use.

Two C57BL/6JOlaHsd female, 8-week-old mice, were used in this study. Mice were purchased from Envigo and weighed 19-20 gr. One male mutant mouse (Ja18 KO mutation that prevents expression of the invariant natural killer T cell T cell receptor (NKT TCR)) was used from a colony that is maintained in the Oxford University Biomedical animal facility.

For Fluorescence Activated Cell Sorting (FACS), the following were used: Phosphate-buffered saline (PBS), RPMI, Full medium: (RPMI with 10% Feotal Bovine Serum (FBS), Penicillin (25,000U) Streptomycin (25 mg/L), glutamine (2.1 mM), (all Tissue Culture grade products from Gibco); Red Blood Cell Lysis Solution (Qiagen). All antibodies were purchased form eBioscience or Biolegend: CD11b-FITC, CD105-BV421, CD45.2-APC, TCRpanBeta-APC-cy7, PD-L1-PE, CD69-PEcy7, (from Biolegend), and Live/Dead fixable stain (ThermoFisher scientific). Fc blocking antibody anti-CD16/CD32 (eBioscience)

Both anti-PD-1 (RMPI-14) and isotype control (2A3) were diluted from the stock down to 400 μg/ml. The antibodies were then further diluted 1:4 to 100 μg/ml which was the final concentration in the well.

To obtain splenocytes, mice were euthanized by cervical dislocation, and spleens were collected and kept in 1.5 ml RPMI on ice. Spleens were mashed and squeezed through a 70 μm sieve into a 50 ml Falcon tube using the flat end of a 1 ml syringe plunger, and the cells were pelleted by centrifugation at 1500 rpm for 5 minutes in an under-bench centrifuge (484 g). Splenocytes were resuspended in 2 ml Red Cell Lysis Solution (Qiagen), and after 2 minutes at room temperature were diluted with 8 ml of full media (RPMI, 10% FBS, Pen/strep, Glut). Splenocytes were washed and then pelleted by centrifugation (484 g) at room temperature. Splenocyets were then resuspended in 3mls of full media and counted. Cells were resuspended at 5×1e7/ml and 100 μl (5×1e6) aliquots of the splenocytes were placed in a well of a plate of 9U bottom wells.

### Assay

100 μl of splenocytes were placed in wells of a 9U-bottom well plate. Antibodies RMPI-14 and 2A3 were diluted to 4-fold of the final concentration (400 μg/ml) and added as 50 μl to each well to achieve a final concentration of 100 μg/ml.

Cells were incubated for 10 minutes at room temperature before adding the iNKT cell agonists, to allow the antibodies to bind.

An additional 50 μl of full media containing ×4 concentration of final concentration of IMM60, IMM47 or Vehicle were added. Final effective concentrations of IMM60 or IMM47 of 0.0, 0.0001, 0.001, 0.01, 0.1, 1.1, 11, and 110 ng/ml were assayed.

Plates were moved to a 37° C., 5% CO₂ incubator for 48 hours. Plates were then removed, washed, and treated with purified anti-mouse CD16/CD32 (1:50 dilution Biolegend) and then washed and stained with fluorescent antibodies and live stain as summarized in the following table:

FACS analysis of splenocytes.

| | PANEL FOR | | |
|---|---|---|---|
| | DENDRITIC | | |
| FLUORESCENCE | CELLS | MARKER | DILUTION |
| FITC | CD11b | Myeloid cell | 1:200 |
| | | population | |
| PE | PD-L1 | molecule up | 1:200 |
| | | regulated in response | |
| | | to INF-gamma and | |
| | | also involved in | |
| | | inhibition of PD-1 + | |
| | | T cells | |
| BV421 | CD105 | Distinguishes | 1:200 |
| | | B16f10 cells from | |
| | | splenocytes | |
| APC | CD45.2 | Immune cell | 1:400 |
| APC-Cy7 | TCR pan Beta | T cell | 1:200 |
| PE-Cy7 | CD69 | T cell activation | 1:200 |
| Aqua(510 nm) | Live/Dead | Viability | 1:200 |

Cell were pelleted by centrifugation for 2 minutes at 1800 rpm (577 g), and the supernatant was discarded. Each pellet was resuspended in a mixture of fluorescent antibodies and left on Ice for 20 minutes, and then washed twice in PBS+0.1% FBS before being diluted with 200 μl of the same solution and analyzed using BD LSR Fortessa FACS machine. Finally, data was analyzed using FlowJo software (CricketGraph).

For B16f10, live cells were gated followed by CD45.2-negative (non-immune cells) CD105-positive (aB16f10 marker). A final gate was set to focus on the main B16f10 population based on forward and side scatter (indicators of size and granularity, respectively). Finally, PD-L1 levels were assessed on that final gated population. Data from FACS analysis of splenocytes was captured using BD LSR Fortessa and was then analyzed using FlowJo software. Finally, GraphPad's Prism statistical analysis software was used to plot and analyze the data.

### Results

PD-L1 Levels on B16f10 Cells Increase with Treatment with Higher Concentrations of IMM60.

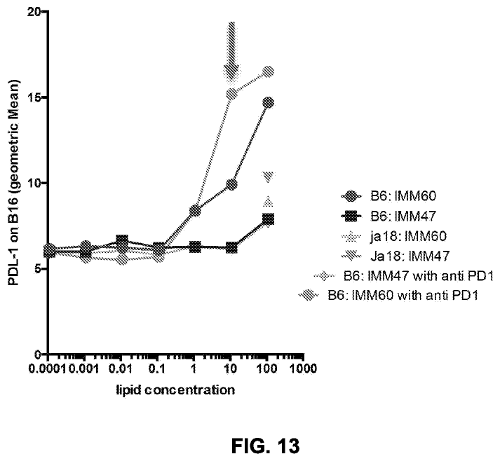

Increasing the concentrations of IMM60 in co-culture of B16f10 with splenocytes from C57BL/mice resulted in increased expression of PD-L1 on B16f10 cells. This increase was not observed when using vehicle alone or when the IMM60 was added to of B16f10 co-cultured with splenocytes from Ja18 KO mice, which do not have any iNKT cells. (FIG. 13) Treatment with a combination of IMM60 and anti-PD-1 increases the level of surface PD-L1 further.

In a parallel set of experiments, co-cultured B16f10 cells and splenocytes were cultured with varying concentrations of IMM60 and IMM47, also supplemented with antibodies (100 μg/ml), either anti-mouse PD-1 (clone RMPI-14) or an isotype control antibody (clone 2A3). FIG. 13 shows a slight increase of PD-L1 expression when anti-PD-1 antibodies where used compared with isotype control antibodies. This increase was only seen in co-cultures at and above 11 ng/ml IMM60 (indicated by the arrow).

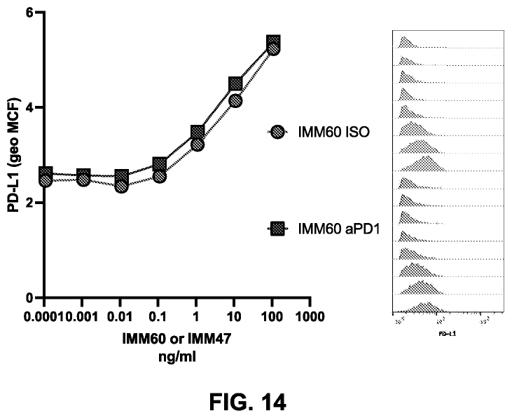

T cells (identified using a TCRpanBeta antibody) in the same B16f10 cells:splenocytes co-culture also upregulate surface PD-L1 when IMM60 was added. However, unlike in the B16f10 cells, this upregulation was not affected by the presence of an anti-PD-1 antibody (FIG. 14).

### Conclusions

The results of this experiment show that in vitro co-cultured splenocytes contain a sufficient number of iNKT cells to exert an agonist-specific effect on B16f10 melanoma cells that were co-cultured with them. This effect is likely somewhat muted compared with the effect that can be seen in vivo. In vivo, we have observed an IFN-gamma response using as little as 0.01 ng/mouse when injected intravenously. See Jukes J P et al. J. Eur. Immunol. (2016) 46:1224-1234.

Non-glycosidic compounds can stimulate both human and mouse iNKT cells. It is quite possible that such sensitivity, compared with the in vitro assay, is due to efficiency of capture of the IMM60 in the live mouse and the efficient delivery to the iNKT cells. Such efficiency of capture and delivery has been demonstrated in a paper discussing the capture of blood-borne antigens by CD169+ marginal zone macrophages and dendritic cells which can present lipids to iNKT cells. See Barral et al EMBO J. (2012). 31:2378-2390. The location of splenic NKT cells favours their rapid activation by blood-borne antigen.

The results of this study also show that the IMM60-induced PD-L1 upregulation on the surface of B16f10 cells is further enhanced by treatment with an anti-PD-1 antibody.

Surface PD-L1 is known to be upregulated by the presence of IFN-gamma and therefore may be a phenotypic surrogate for the presence of IFN-gamma. However, the involvement of PD-L1 in inhibition of PD-1+ T cells, and the effect of blocking such interaction that is observed in this study, shows that a negative feedback loop may be disrupted by the anti-PD1 antibody, although the data of this study cannot determine if it is the PD-L1 on the B16f10 cells that causes this negative feedback.

On T cells, the up regulation of surface PD-L1, which was evident when IMM60 was added, was not enhanced by anti-PD1 antibody treatment. It is therefore likely that the disruption of the PD-1:PD-L1 axis and its effect on further upregulation of PD-L1 is very local, and perhaps cognate and bystander cells such as conventional T cells are not affected.

### Example 12: Clinical Trial Protocol

Following is a clinical trial protocol for administration if IMM60 alone or as part of a combination therapy. The dose and dosing regimens described herein are contemplated as specific embodiments. The protocol described can be repeated/modified with different liposomal formulations of IMM60; and can be repeated/modified with an alternative secondary therapy, such as a different anti-PD-1 antibody or a different immune checkpoint inhibitor.

### IMM60 and Anti-PD1

The combination of IMM60 plus anti-PD1 treatment in mice results in enhanced IFNγ secretion in a dose dependent manner over IMM60 alone. This results in further activation of NK cells and greater production of IFN-γ secretion. In addition, anti-PD1 antibodies block PD1 expressed on the surface of iNKT cells, resulting in enhanced maturation of dendritic cells and B-cells and promotion of priming/boosting of antigen-specific CD8+ T-cells. In animals that are resistant to anti-PD1 therapy alone, the combination of IMM60 plus anti-PD1 antibody restores sensitivity.

### Drug Supplies and Dispensing

Pembrolizumab is an anti-PD-1 antibody marketed by Merck under the trade name Keytruda®. Pembrolizumab Solution for Infusion is a sterile, non-pyrogenic aqueous solution supplied in a single-use Type I glass vial containing 100 mg/4 ml of pembrolizumab. Pembrolizumab infusion solutions should be prepared by site pharmacy in 0.9% Sodium Chloride (normal saline) and the final concentration of pembrolizumab in the infusion solution should be between 1 mg/ml and 10 mg/ml. For intravesical infusions the final volume of infusion instilled will be 40 ml.

In some embodiments, IMM60 is formulated in liposomes as described herein. IMM60 should be stored frozen, then thawed and diluted by the site pharmacy prior to dispensing to the ward. The recommended dose of liposomal IMM60 is either 1, 3, or 9 mg/m² administered by intravenous infusion over 60 minutes in a 250 ml Macopharma saline diluent, every 3 weeks. IMM60 is supplied as a 1 mg/ml solution, with 2 ml in each vial and should be stored at −20° C. and will need to be thawed at pharmacy prior to dispensing. Prior to use, vials are allowed to thaw for 1 to 2 hours until ambient temperature is reached. Heat vials in a 55° C.±2° C. water bath for 10 minutes. Invert and swirl the vials several times by hand and allow to cool to room temperature prior to use.

### Rationale for the Study

This clinical trial provides a protocol to demonstrate a synergistic effect of IMM60 and pembrolizumab, compared to pembrolizumab alone in patients with melanoma and non-small cell lung cancer, for which pembrolizumab was already licensed as a standard of care. The trial evaluates if IMM60 can restore sensitivity to patients with melanoma resistant to pembrolizumab as well as ascertain if IMM60 can promote PDL1 expression in PDL1 non-expressing small cell lung cancer. The clinical trial includes two phases, phase 1 and phase 2. The clinical trial is executed following the protocol in Table CT-1.

Initial safety will be assessed in a multiple ascending dose cohort for IMM60, then for IMM60+pembrolizumab.

TABLE CT-1

| Full Title | A phase 1 first-in-human dose finding/ | |
|---|---|---|
| of study: | randomised phase 2 study of IMM60 | |
| | and pembrolizumab in advanced melanoma | |
| | and Non-small cell lung cancer | |
| | (NSCLC)NSCLC | |
| Study Design: | Phase 1: 3 + 3 dose escalation; Phase 2: Randomised 1:2 | |
| | Objectives | Endpoints |
| Primary | Phase 1: To characterize | Frequency of grade |
| Endpoint: | the safety and confirm the | 3 or higher treatment |
| | maximum tolerated | related adverse events |
| | dose (MTD) of IMM60 | (AEs). |
| | alone and in combination | Maximum |
| | with pembrolizumab, in | tolerated dose |
| | patients with advanced | |
| | malignant melanoma | |
| | or PDL1 +ve (PDL1 | |
| | ≥50%) NSCLC | |
| | Phase 2: To report the | Progression free |
| | progression free survival | survival (PFS) at 12 |
| | rate at 12 months for | months |
| | pembrolizumab alone | |
| | versus IMM60 + | |
| | pembrolizumab in | |
| | patients with | |
| | advanced PDL1 +ve | |
| | (PDL1 ≥50%) | |
| | NSCLC or Melanoma | |
| Secondary | To characterize the | Frequency of |
| Endpoints: | safety of IMM60 | grade 3 or higher |
| | in combination | treatment related AEs. |
| | with pembrolizumab, | |
| | PDL1 +ve (PDL1 | |
| | ≥50%) NSCLC | |
| | Pharmacokinetics | IMM60 Cmax and AUC |
| | of IMM60 (Phase | IFN-γ in the blood |
| | 1 and Phase 2, in IMM60 | |
| | arms only) | |
| | To determine if IMM60 | Objective response |
| | can restore sensitivity | rate (ORR) in |
| | in PD1 inhibitor resistant | melanoma patients |
| | melanoma (Phase 2) | who have progressed |
| | | on PD1, and have added |
| | | IMM60 |
| | To determine if IMM60 | ORR by RECIST 1.1 |
| | can sensitize patients with | in PDL1-ve NSCLC |
| | PDL1-ve (PDL1 <50%) | patients who receive |
| | NSCLC to PD1 inhibition | IMM60 + Pembrolizumab |
| | (Phase 2) | |
| | Objective response | ORR by RECIST |
| | rate (ORR − CR | 1.1 (Best Overall |
| | & PR by RECIST | Response is defined |
| | 1.1) in all groups | as RECIST 1.1 response |
| | (Phase 1 and Phase 2) | rate including confirmed |
| | | CR and PR) |
| Planned | Phase 1 (patients with advanced NSCLC or Melanoma): | |
| enrollment: | 1. IMM60 dose escalation arm: 12 | |
| | 2. IMM60 + pembrolizumab safety arm: 6 | |
| | Upon review of the Phase 1 data the | |
| | following cohorts may be activated by | |
| | the Trial Management Group (TMG) | |
| | Phase 2 randomized 1:2 in melanoma, | |
| | “Melanoma Cohort 1”: | |
| | 1. Pembrolizumab alone: 7 | |
| | 2. IMM60 + pembrolizumab: 14 | |
| | Additional melanoma cohort “Melanoma Cohort 2”: | |
| | 3. IMM60 alone: 10 | |
| | Phase 2 randomized 1:2 in NSCLC, “NSCLC Cohort 1”: | |
| | 1. pembrolizumab alone in PDL1 + NSCLC: 12 | |
| | 2. pembrolizumab + IMM60 in PDL1 + NSCLC: 24 | |
| | Potential Additional NSCLC cohort pending | |
| | phase 1 data review, “NSCLC | |
| | Cohort 2”: | |
| | 3. IMM60 followed by IMM60 + | |
| | Pembrolizumab in PDL1-ve | |
| | NSCLC:15 | |
| | Total: 100 | |
| Target | Phase 1: | |
| Population: | IMM60 dose escalation arm: | |
| | NSCLC | |
| | Progression through systemic therapy | |
| | consisting of at least platinum based | |
| | chemotherapy and immunotherapy (either | |
| | sequentially or in combination) | |
| | Melanoma | |
| | Patients progressing through at least one line of | |
| | immunotherapy. For those patients with BRAF mutant | |
| | tumours, progression through BRAF/MEK inhibitor | |
| | treatment and at least one line of immunotherapy | |
| | IMM60 + pembrolizumab dose safety arm: | |
| | NSCLC | |
| | First line | |
| | PDL1 ≥50% | |
| | Meeting all CDF funding criteria for pembrolizumab | |
| | prescribing apart from administration as monotherapy | |
| | Melanoma | |
| | Not previously exposed to PD-1 inhibitors | |
| | Meeting all CDF funding criteria for pembrolizumab | |
| | prescribing apart from administration as monotherapy | |
| | Phase 2: | |
| | Melanoma arms: | |
| | Melanoma Cohort 1: Patients with metastatic | |
| | disease not previously exposed | |
| | a PD-1 inhibitor | |
| | Melanoma Cohort 2: Patients with metastatic | |
| | disease previously exposed to | |
| | a PD-1 inhibitor | |
| | Patients in Melanoma Cohorts 1 must | |
| | meet CDF criteria for funding for | |
| | pembrolizumab. Melanoma Cohort 2 | |
| | patients do not need to meet this | |
| | criteria (IMM60 administration as monotherapy) | |
| | Non-small cell lung cancer (NSCLC) arms: | |
| | NSCLC Cohort 1: Metastatic NSLC, | |
| | PDL1 +ve (PDL1 ≥50%) not | |
| | previously exposed to a PD-1 inhibitor | |
| | NSCLC Cohort 2: Metastatic NSLC, | |
| | PDL1 −ve (PDL1 <50%) not | |
| | previously exposed to a PD-1 inhibitor | |
| | but pre-treated with at least one line | |
| | of systemic treatment | |
| | Patients in NSCLC Cohorts 1 must meet | |
| | CDF criteria for funding for | |
| | pembrolizumab, with the exception of | |
| | pembrolizumab administration as | |
| | monotherapy | |
| | Name of drug | Formulation, dose, route |
| | | of administration |

| | |
|---|---|
| Investigational | IMM60 & pembrolizumab (doses |
| Medicinal | and schedule as per institution standard of |
| Product(s) | care) |
| Treatment | IMM60 is given in 3-weekly cycles |
| Duration | with a maximum of cycles, |
| | pembrolizumab as per its licensed indication |
| Follow-up | Up to 1 year from first dose of treatment or |
| duration | until disease progression or death, |
| | or, whichever is sooner. Beyond this |
| | point, any patients continuing |
| | pembrolizumab under the National |
| | Health Science (NHS) license will be |
| | monitored for 1 year or until disease |
| | progression or death, whichever is |
| | sooner. |
| End of study | Once last patient has completed follow-up |
| | period (including further 1 year |
| | period if continuing on pembrolizumab |
| | under NHS license), or until disease |
| | progression or death, whichever is sooner. |

TABLE CT-2
Summary of schedule of events in the trial

| | Visit | | | | | | | | | | | | |
|---|---|---|---|---|---|---|---|---|---|---|---|---|---|
| | 1 | 2 | 3 | 4 | 5 | 6 | 7 | 8 | 9 | 10 | Off Treatment Visits | | |
| | Cycle | | | | | | | | | | | | |
| | 0 | 1 | 1 | 1 | 2 | 2 | 3 | 4 | 5 | 6 | N/A | N/A | N/A |
| | Day | | | | | | | | | | | | |
| | | | | | | | | | | | | 30 days | Every 3 |
| | −14 to −1 | 1 | 8 | 15 | 22 | 29 | 43 | 64 | 85 | 106 | N/A | post Tx | months |
| | Visit description | | | | | | | | | | | | |
| | | | | | | | | | | | Stop | Post- | |
| | Main study | | | | | | | | | | Treatment | Treatment | Follow |
| | screening | Baseline | Treatment | | | | | | | | Visit | Visit | up |
| | Visit window(days) | | | | | | | | | | | | |
| | | N/A | +/−2 | +/−2 | +/−2 | +/−2 | +/−2 | +/−7 | +/−7 | +/−7 | | | |
| Main study consent | X | | | | | | | | | | | | |
| Demographics | X | | | | | | | | | | | | |
| Medical History | X | | | | | | | | | | | | |
| Concomitant Treatments | X | X | X | | X | | X | X | X | X | X | X | |
| ECOG⁹ PS | X | X | X | | X | | X | X | X | X | X | X | |
| Physical Exam | X | X | X | X | X | X | X | X | X | X | X | X | |
| Vital Signs | X | X | X | X | X | X | X | X | X | X | X | X | |
| Height | X | | | | | | | | | | | | |
| Weight | X | | | | X | | X | X | X | X | X | X | |
| Haematology and | X | X | X | X | X | X | X | X | X | X | X | X | |
| Biochemistry | | | | | | | | | | | | | |
| PDL1 status (NSCLC | X | | | | | | | | | | | | |
| patients only) | | | | | | | | | | | | | |
| ECG | X | X | | | | | | | | | | | |
| Pregnancy test | X | X | | | X | | X | X | X | X | | X | |
| Pembrolizumab¹ | | X | | | X | | X | X | X | X | | | |
| IMM60⁴ | | X | | | X | | X | X | X | X | | | |
| Urinalysis | X | X | X | X | X | X | X | X | X | X | X | X | |
| Adverse Events | X | X | X | X | X | X | X | X | X | X | X | X | |
| CT scan | Baseline | | | | | | | | X | | X | | X |
| MRI Brain Scan | Baseline | | | | | | | | X | | | | X |
| PK blood sample | | X | | | | | | | | | | | |
| Research Blood sample | | X | X | | | | | | | | | | |
| Biopsy | X | | | | X | | | | | | | | |

¹IMM60 or pembrolizumab or IMM60 and pembrolizumab administered depending on cohort.

### Justification for Dosing

In accordance with ICH S9, Nonclinical Evaluation for Anticancer Pharmaceuticals, the clinical starting dose is to be set as human equivalent dose (HED) the lower of either 1/10 of the STD10 in rodents or 1/the highest non-severely toxic dose (HNSTD) in non-rodents. For liposomal IMM60, the STD10 in mice was >5 mg/kg (15 mg/m²), as there were no deaths, clinical signs, or decreases in body weight at this dose. Humans are expected to be much less sensitive than mice to the toxicological effects of liposomal IMM60 since these are likely mediated through exaggerated pharmacological effects (excessive IFN-γ production). Therefore, the estimated clinical starting dose conservatively based upon nonclinical safety studies in the mouse would be 1/10 the STD10 in mice or 1.5 mg/m². The actual planned clinical dose is set at 1.0 mg/m², a dose that is lower than 1/10 the STD10 in mice (1.5 mg/m²).

### Trial Design

This will be a randomized phase 1/2 trial. Initial safety will be assessed in a multiple ascending dose cohort for IMM60, then for IMM60+pembrolizumab. The Trial Management Group (TMG) will review the Phase 1 data, the current standard of care and feasibility to make a recommendation on Phase 2 cohorts. Planned arms in the Phase 2 component include: pembrolizumab alone & IMM60+pembrolizumab for NCSLC patients; and pembrolizumab alone, IMM60+pembrolizumab, and additional IMM60 alone cohort for melanoma patients.

The Phase 1 trial is a modified 3+3 trial design. In the IMM60 ascending dose safety arm, three dose levels of IMM60 were assessed (1/3/9 mg/m²). If one of the first 3 patients experiences a pre-defined dose limiting toxicity (DLT), that cohort is expanded to a total of patients. If a further patient in this cohort experiences a DLT, this dose level is defined as the maximum tolerated dose (MTD). If more than 2 DLTs occur in the patients in the expanded cohort, then MTD will be defined as the dose level below that cohort. Should more than 2 DLTs occur in the lowest IMM60 dose level cohort, then de-escalating this dose level is contemplated. If no patient in the highest dose cohort (9 mg/m²) experiences a DLT, this cohort will still be expanded to patients. To decide if the next cohort can be opened at a higher dose level in Phase 1 of the study, the Trial Management Group (TMG) will review available data (e.g., safety profile) once all participants in the preceding cohort have completed the first cycle through to Day 21. Subsequent safety reviews by the TMG will be conducted prior to enrolment of patients in a new cohort.

For the IMM60+pembrolizumab safety arm, pembrolizumab will be administered in combination with IMM60. The dose of pembrolizumab will be that administered as per standard of care; the IMM60 dose will be dependent on the IMM60 dose escalation arm. This combination safety arm will also use the modified 3+3 design—the IMM60 starting dose level will be one below IMM60 MTD (referred to as MTD-1), and will dose escalate to a cohort with IMM60 at MTD. For example, if the IMM60 MTD is 9 mg/m², the first combination cohort will be with IMM60 at 3 mg/m² and will escalate to a combination cohort with IMM60 at 9 mg/m². Should the IMM60 MTD be 1 mg/m² (lowest dose level in the ascending dose cohort), then the TMG will consider whether to begin the combination safety arm at MTD, or a de-escalated dose. The DLT rules applied to the IMM60 dose escalation arms will also apply to the combination safety arms.

For Phase 2 of the trial, patients with melanoma will be randomised 1:2 between treatment arms: either pembrolizumab as per its licensed indication or the combination of pembrolizumab+IMM60. Another additional contemplated cohort is IMM60 at the MTD given every 3 weeks for patients who have failed pembrolizumab. Potential patients with PDL1-positive NSCLC will be randomised 1:2 to either pembrolizumab as per its licensed indication or the combination of pembrolizumab+IMM60. Depending on review of IMM60 monotherapy activity, both melanoma and NSCLC patients in the pembrolizumab alone cohort who show no response at their 3-month CT scan can then receive pembrolizumab+IMM60.

An additional comteplated cohort of PDL1—NSCLC is treated with one cycle of IMM60, with a tumour biopsy before and after, to determine any changes in PDL1 expression. After this one cycle, the patients will receive the combination of IMM60+pembrolizumab and a second biopsy will be taken. Depending on review IMM60 monotherapy activity, a potential group of patients who are receiving pembrolizumab monotherapy will be allowed to receive IMM60 at progression in combination with the pembrolizumab to see if sensitivity can be restored.

### Dose Escalation Procedure

An evaluable patient is defined as a patient who has received the Cycle 1 dose and has completed the minimum safety evaluation requirements in the 21-day period, or experienced a DLT in the 21-day DLT evaluation period. Dose escalation decisions will be made when 3 evaluable patients in a cohort have completed the 21-day DLT evaluation period; should a cohort need to be expanded to patients due to a DLT, the dose-escalation decision will be made once evaluable patients have completed their 21-day DLT period. Dose escalation will occur based on the toxicity information obtained from the evaluable patients in a cohort during the initial 21-day treatment cycle. Dose escalation guidelines for the Phase 1 cohorts are indicated in Table CT-3 below.

TABLE CT-3
IMM60 Dose Escalation Safety Arm

| | | | No additional | | |
|---|---|---|---|---|---|
| Dose/Arm | No DLT | 1x DLT | DLT | 2x DLT | 3+ x DLT |
| 1 mg/m² | Dose escalate | Expand to | Dose escalate to | 1 mg/m² is | TMG to |
| IMM60 | to 3 mg/m² | patients | 3 mg/m² | IMM60 MTD | consider dose |
| | | | | | de-escalation |
| 3 mg/m² | Dose escalate | Expand to | Dose escalate to | 3mg/m² is | 1 mg/m² is |
| IMM60 | to 9 mg/m² | patients | 9 mg/m² | IMM60 MTD | IMM60 MTD |
| 9 mg/m² | Expand to | Expand to | IMM60 MTD | 9/mg/m² is | 3 mg/m² is |
| IMM60 | patients | patients | | IMM60 | IMM60 MTD |
| | | | | MTD | |

IMM60 + pembrolizumab Combination Safety Arm

| | | | No additional | | |
|---|---|---|---|---|---|
| Dose/Arm | No DLT | 1x DLT | DLT | 2x DLT | 3+ x DLT |
| IMM60 MTD-1 | Dose escalate to | Expand to | Dose escalate to | MTD-1 is | TMG to |
| + | IMM60 | patients | MTD + | dose for | consider dose |
| pembrolizumab | MTD | | pembrolizumab | phase 2 | de-escalation* |
| IMM60 MTD | IMM60 dose | Expand to | IMM60 dose for | MTD is | MTD-1 is dose |
| + | for Phase 2 | patients | Phase 2 | dose for | for phase 2 |
| pembrolizumab | | | | phase 2 | |

### Procedure for Opening Phase 2

Interim analysis will be conducted on the Phase 1 cohorts and reviewed by the TMG once all patients in the Phase 1 cohorts have completed their DLT period to determine whether the trial will proceed to Phase 2 and to also determine which cohorts will be opened. The analysis will be based solely on safety data, and a review conducted for the most severe toxicities. The following stopping rules will be applied; should either of the below occur then the trial will not proceed to Phase 2: If the rate of DLTs exceed 33% or if the rate of treatment-related serious adverse events (SAEs) exceeds 50%. The TMG will review pharmacodynamic data with the tissue and blood biomarkers and also review the data for efficacy, specifically, disease stabilization and evidence of immune activation.

Duration of patient participation: Patients receiving IMM60 can receive a maximum of cycles, given at 3-weekly intervals, and patients are to attend a post-treatment visit 1 month after their final dose. Patients receiving pembrolizumab will be in the study for a maximum of 1 year and 1 month from first trial dose to last protocol visit. Patient duration on trial is dependent on their treatment.

Post-trial care and follow-up: Following the Post-Treatment visit, patients will receive standard care. Patients will be followed up for progression-free survival and overall survival data via the medical care team for the duration of the follow-up period (up to 1 year) or until disease progression or death, whichever is sooner.

### Objectives and Endpoints for the Clinical Trial are Laid Out in Table CT-4.

TABLE CT-4

| | | Time point(s) of |
|---|---|---|
| Primary Objective | Endpoints/ | evaluation of this |
| (Phase 1) | Outcome measures | end point |
| To characterize the | Frequency of grade | During dose |
| safety and confirm the | 3 or higher | escalation period |
| MTD of IMM60 alone | treatment related | End of phase 1 |
| and in combination with | AEs. | dose escalation |
| pembrolizumab, in patients | Maximum tolerated | Completion of |
| with advanced malignant | dose | phase 1 safety |
| melanoma or PDL1 + ve | | cohort |
| (PDL1 ≥50%) NSCLC | | |
| | | Time point(s) of |
| Primary Objective | Endpoints/ | evaluation of this |
| (Phase 2) | Outcome measures | end point |
| To report the progression | Progression free | 12 months |
| free survival | survival at 12 | |
| rate at 12 months for | months (PFS) | |
| pembrolizumab alone | | |
| with IMM60 + | | |
| pembrolizumab in patients | | |
| with advanced | | |
| PDL1 + ve NSCLC or | | |
| Melanoma | | |
| Secondary Objectives | | |
| (Phase 1 & 2) | Endpoints | |
| Pharmacokinetics of IMM60 | IMM60 Cmax | 1, 2, 4, 6, 24 hours |
| (Phase 1 and Phase 2, | and AUC | post first dose |
| in IMM60 arms only) | IFN-γ in the blood | |
| To characterize the | Frequency of | months |
| safety of IMM60 | grade 3 or higher | |
| in combination with | treatment related | |
| pembrolizumab, | AEs. | |
| PDL1 + ve (PDL1 | | |
| ≥50%) NSCLC | | |
| (Phase 2) | | |
| To determine if | ORR in melanoma | months |
| IMM60 can restore | patients who | |
| sensitivity in PD1 | have progressed | |
| inhibitor resistant | on PD1, and have | |
| melanomas (Phase 2) | added IMM60 | |
| RECIST 1.1 Objective | ORR by RECIST 1.1 | 12 months |
| response rate | (Best Overall | |
| (ORR) in all groups | Response is defined | |
| (Phase 1 & 2) | as RECIST 1.1 | |
| | response rate | |
| | including confirmed | |
| | CR and PR) | |
| To determine if IMM60 | ORR in PDL1-ve | months |
| can sensitize | NSCLC patients | |
| patients with PDL1-ve | who receive | |
| NSCLC to PD1 | IMM60 + | |
| inhibition (if this | Pembrolizumab | |
| cohort is added to | | |
| the Phase 2) | | |
| Tertiary/Exploratory | | |
| Objectives | Endpoints | |
| To assess | Immuno- | Blood and tumour |
| pharmacodynamics and | histochemistry | samples at |
| the impact of | Flow cytometry | baseline, |
| tumour characteristics | T-cell repertoire | at 3 weeks |
| on response to IMM60 | CYTOF analysis | and at weeks |
| therapy with or | | |
| without pembrolizumab | | |
| To assess the ability | % of PDL1-ve | Biopsy Samples at |
| of IMM60 to | NSCLC tumours | baseline and at 3 |
| convert PDL1-ve | that develop | weeks |
| patients to | PDL1 expression of | |
| PDL1+ve | ≥50% following | |
| | treatment with | |
| | IMM60 | |

### Evaluation of Response

Response Evaluation Criteria in Solid Tumours (RECIST): All patients must have at least 1 lesion that is measurable using RECIST 1.1 in a field that has not previously been treated by radiotherapy.

Tumour Assessment: Radiological evaluation of malignancy, in line with the protocol, will be performed before starting the study treatment. The same methods that detect lesions at baseline will be used to follow these lesions throughout the study. To ensure compatibility, the radiological assessments used to assess response will be performed using identical techniques. Imaging based evaluation is preferred to evaluation by clinical examination when both methods have been used to assess the anti-tumour effect of a treatment.

Baseline evaluations: These will include radiological measurements of the extent of disease by CT scan, or MRI scan where applicable. All areas of disease present must be mentioned (even if specific lesions are not going to be followed for response) and the measurements of all measurable lesions must be recorded on the scan reports. Any non-measurable lesions must be stated as being present.

Evaluations during treatment and at off-study: Tumour assessment will be repeated as per the schedule given herein or more frequently if clinically indicated. All lesions measured at baseline must be measured at subsequent disease assessments, and recorded on the scan reports. All non-measurable lesions noted at baseline must be reported as present or absent.

Radiological evaluation of malignancy, in line with the protocol, must be performed before starting the study treatment. The same methods that detect lesions at baseline will be used to follow these lesions throughout the study. To ensure compatibility, the radiological assessments used to assess response must be performed using identical techniques. Imaging based evaluation is preferred to evaluation by clinical examination when both methods have been used to assess the anti-tumour effect of a treatment.

The Principal Investigator must ensure that the radiologists are aware of the requirement to follow up and measure every target lesion mentioned at baseline and comment on the non-target lesions in accordance with RECIST criteria. Patients continuing pembrolizumab under the NHS license will be monitored for 1 year or until disease progression or death, whichever is sooner. During this period, disease assessments will not be required to use RECIST, and routine reporting will be sufficient.

Tumour Response: To be assigned a status of complete response (CR) or partial response (PR), changes in tumour measurements must be confirmed by two consecutive observations, at least 4 weeks apart. To be assigned a status of stable disease (SD), follow-up measurements must have met the SD criteria at least once and at least six weeks after study treatment is started. Should rapid tumour progression occur before the completion of 9 weeks treatment, the patient will be classified as having early progression (EP).

Tumour response should be classified as “not evaluable” (NE), only when it is not possible to classify it under another response category, e.g., when baseline and/or follow-up assessment is not performed or not performed appropriately.

The applicable overall response category for each visit that includes disease assessment must be recorded in the medical record for inclusion in the appropriate CRF in OpenClinica.

Best Overall Response is defined as RECIST 1.1 response rate including confirmed CR and PR.

Preliminary results from the IMM60 ascending dose safety arm are shown in Tables CT5 and CT6, wherein nine patient received infusions of IMM60 at three doses (1, 3, or 9 mg/m²) via infusion over a period of about 1 hour.

TABLE CT5
Pharmacokinetic Properties

| | | | Plasma concentrations | | | | | | | MRT with | |
|---|---|---|---|---|---|---|---|---|---|---|---|
| | Dose | Actual | 1 h | 2 h | 4 h | 6 h | 24 h | | t_{1/2} | infusion | Clearance |
| Patient | (mg/m²) | Dose | (ng/mL) | (ng/mL) | (ng/mL) | (ng/mL) | (ng/ml) | k^{b} | (h) | correction (h) | (mL/h) |
| 1^{a} | 1 | 2.1 | 25 | 37 | 34 | 19 | 2 | 0.117 | 6 | 4.7 | 5569 |
| 2 | 1 | 1.5 | 260 | 133 | 180 | 346 | 74 | 0.086 | 8 | 7.9 | 258 |
| 3 | 1 | 2.1 | 142 | 122 | 121 | 180 | 21 | 0.119 | 6 | 6.2 | 767 |
| 4 | 3 | 5.4 | 426/142 | 405/135 | 528/176 | 361/120 | 115/38 | 0.064 | 11 | 9.0 | 632 |
| 5^{a} | 3 | 5.7 | 181/60 | 295/98 | 254/85 | 179/60 | 35/12 | 0.090 | 8 | 6.0 | 1571 |
| 6 | 3 | 6.3 | 660/220 | 638/213 | 887/296 | 849/283 | 191/64 | 0.083 | 8 | 7.5 | 396 |
| 7 | 9 | 18.5 | 955/106 | 1,970/219 | 1,800/200 | 1,990/221 | 673/75 | 0.060 | 12 | 9.8 | 414 |
| 8 | 3 | 6.0 | 776/259 | 554/185 | 864/288 | 960/320 | 185/62 | 0.091 | 8 | 7.3 | 361 |
| 9 | 9 | 17.0 | 3,390/377 | 1,740/193 | 960/107 | 457/51 | 128/14 | 0.071 | 10 | 5.8 | 1100 |
| | | | | | | | | avg | 8 | | |
| | | | | | | | | CV | 0.238 | | |

^{a}infused over a period of about 2 hours due to administration complications, whole dose may not have been given.
^{b}terminal elimination rate constant calculated using t(6 h) and t(24 h) plasma values.

TABLE CT6
Pharmacokinetic Properties

| | | | | | | | | | AUC Multiple | Cmax Multiple |
|---|---|---|---|---|---|---|---|---|---|---|
| | | | AUC_{inf}/ | | | | Cₘₐₓ/ | | over 50 | 50 ug/kg |
| | AUC_{24 h} | AUC_{inf} | dose | AUC_{inf}/ | Vdₜₑᵣₘ^{b} | Vdₛₛ^{c} | dose | Cₘₐₓ/ | ug/kg mid | mid tox |
| Patient | (ng/mL/h) | (ng/mL/h) | group | dose | (mL) | (mL) | group | dose | tox group | group |
| 1^{a} | 358 | 377 | 377 | 180 | 47,494 | 32487 | 37 | 17.7 | 2 | 0 |
| 2 | 4,946 | 5,809 | 5809 | 3873 | 3,013 | 2175 | 346 | 230.7 | 23 | 2 |
| 3 | 2,558 | 2,736 | 2736 | 1303 | 6,459 | 5149 | 180 | 85.7 | 11 | 1 |
| 4 | 6,735 | 8,544 | 2848 | 1582 | 9,945 | 5980 | 176 | 97.8 | 34 | 3 |
| 5^{a} | 3,238 | 3,628 | 1209 | 636 | 17,389 | 11153 | 98 | 51.8 | 15 | 2 |
| 6 | 13,600 | 15,905 | 5302 | 2525 | 4,780 | 3230 | 296 | 140.8 | 64 | 5 |
| 7 | 33,467 | 44,641 | 4960 | 2413 | 6,881 | 4378 | 221 | 107.6 | 179 | 10 |
| 8 | 14,600 | 16,622 | 5541 | 2770 | 3,946 | 2802 | 320 | 160.0 | 66 | 5 |
| 9 | 13,642 | 15,452 | 1717 | 909 | 15,560 | 6903 | 377 | 199.4 | 62 | 18 |

^{a}infused over a period of about 2 hours due to administration complications, whole dose may not have been given.
^{b}Volume of Distribution of steady state phase
^{c}Volume of Distribution of terminal elimination phase

As shown in Table CT5, the terminal half-life ranged from about 6-12 hours. The average terminal half-life was 8 h with a linear response with increasing dose. In contrast, the terminal half-life in mice was less than 1 h (e.g., about 47 to about 52 minutes).

The plasma exposure data from the ascending dose arm is summarized in Tables CT5 and CT6 and shows a proportional, linear response to ascending dose. Plasma exposures generally exhibited a plateau from about 1 to about 6 hours. As shown in Table CT6, Patients 2, 3, 4, 6, 7, and 8 exhibited Cₘₐₓ at about 4-6 h post dosing. In contrast, Patients 1, 5, and 9 exhibited Cₘₐₓ earlier (2 h, 2 h, and 1 h, respectively). Without wishing to be bound to any particular theory, it is believed that the variance in plasma exposure may be due to RES/plasma protein interaction.

As shown in Table CT6, the volume distribution during the terminal phase ranged from about 1 to about 3 times the plasma compartment. In mice, this ratio was approximately 3.8 to 5.4 times. Moreover, the exposure by AUC generally was less than 100 times multiple of mid 50 μg/kg dose used in animal toxicology studies. The exception was Patient 7 which exhibited the highest but dose proportion AUC out of the nine patients.

Without wishing to be bound to any particular theory, it is believed that the plasma profile of Patient 10 is qualitatively unique (monotonically decreasing) and exhibited no plateau on plasma concentration between 1 and 6 hours as compared to the other patients. Without wishing to be bound to any particular theory, it is believed that the data herein demonstrates that IMM60 typically exhibits a bimodal plasma profile with most patient with little to no clearance of IMM60 is observed up to about 6 hours while others have a monotonically decreasing plasma profile.

### Example 13: Alternate Clinical Trial Protocol

Referring to Example 12 above, following is another clinical trial protocol for administration if IMM60 alone or as part of a combination therapy. The dose and dosing regimens described herein are contemplated as specific embodiments. The protocol described can be repeated/modified with different liposomal formulations of IMM60; and can be repeated/modified with an alternative secondary therapy, such as a different anti-PD-1 antibody or a different immune checkpoint inhibitor.

PHASE 1

| | Endpoints/Outcome | Time Point(s) |
|---|---|---|
| Primary Objective | Measures | of Evaluation |
| To characterize | Frequency of | During dose |
| the safety | Grade 3 or higher | escalation |
| and confirm | treatment-related | period |
| the maximum | adverse events | End of |
| tolerated dose (MTD) | (AEs) | phase 1 dose |
| of IMM60 alone and | Maximum tolerated | escalation |
| in combination with | dose (MTD) | Completion |
| pembrolizumab, | | of phase 1 |
| in patients with | | safety cohort |
| advanced malignant | | |
| melanoma or | | |
| NSCLC | | |

PHASE 2

| | Endpoints/Outcome | Time Point(s) |
|---|---|---|
| Primary Objective | Measures | of Evaluation |
| To compare the | Progression-free | 12 months |
| progression-free | survival (PFS) at | |
| survival (PFS) | 12 months | |
| rate at 12 months | | |
| in the randomized | | |
| arms comparing | | |
| pembrolizumab | | |
| alone versus IMM60 + | | |
| pembrolizumab | | |
| in patients with | | |
| advanced PDL1 + ve | | |
| NSCLC | | |

PHASE 1 AND 2

| | Endpoints/Outcome | Time Point(s) |
|---|---|---|
| Secondary Objectives | Measures | of Evaluation |
| Pharmacokinetic(s) | IMM60 maximal | 0, 1, 2, 4, 6, |
| (PK) of IMM60 | concentration | 24 hours post |
| (phase 1 and phase | (Cmax) and area | first dose and |
| 2, in IMM60 | under the curve | on Cycle 3 |
| arms only) | (AUC) | Day 1 before |
| | | treatment |
| To characterize | Frequency and | continuous |
| the safety of | severity of | |
| IMM60 alone or in | treatment-related | |
| combination with | AEs | |
| pembrolizumab, | | |
| NSCLC and | | |
| melanoma (phase 2) | | |
| To determine if | Objective response | every 3 months |
| IMM60 can | rate (ORR) in | |
| restore sensitivity | melanoma patients | |
| in PD-1 | who have progressed | |
| inhibitor-resistant | on PD-1, and have | |
| melanoma (phase 2) | added IMM60 | |
| ORR (in all | ORR is to be reported | every 3 months |
| groups [phase 1 | as the proportion of | |
| and phase 2])-ORR by | patients who have a | |
| Response Evaluation | CR or PR | |
| Criteria In Solid | | |
| Tumors (RECIST) | | |
| 1.1 (Best Overall | | |
| Response is defined | | |
| as RECIST 1.1 | | |
| response rate | | |
| including confirmed | | |
| complete response | | |
| [CR] and partial | | |
| response [PR]) | | |
| To determine | Objective response | every 3 months |
| if IMM60 can | rate (ORR) in | |
| sensitize patients | PDL1-ve NSCLC | |
| with PDL1-ve | patients who | |
| NSCLC to PD-1 | receive IMM60 + | |
| inhibition | pembrolizumab | |
| To assess the ability | % of PDL1-ve NSCLC | Biopsy samples at |
| of IMM60 to | tumors that | screening visit |
| convert PDL1-ve | increase PD-L1 gene | and 3 weeks |
| patients to PDL1 + ve | expression following | |
| | treatment with IMM60 | |
| | (additional analysis | |
| | will include 22c3 | |
| | immunohistochemistry | |
| | [IHC] | |
| | expression levels) | |
| Tertiary/Exploratory | Endpoints/Outcome | Time point(s) |
| Objectives | Measures | of Evaluation |
| To assess | Potential analysis | Blood samples at |
| pharmacodynamics | to include: | baseline, at 3 |
| and the | IHC | weeks and |
| impact of tumor | Gene expression | at weeks, and |
| characteristics on | Flow cytometry | tumor at |
| response to IMM60 | T-cell repertoire | baseline and |
| therapy with or | Cytometry by | 3 weeks |
| without | time of flight | |
| pembrolizumab | (CYTOF) analysis | |

### Study Intervention and Intervention Form:

IMM60 liposomes: either 1/3/9 mg/m² administered intravenously (IV) every 3 weeks (Q3W) for cycles; the dose will be calculated for each participant based on body surface area at baseline, and recalculated if a participant's weight changes by >10% during the course of the study. In some variations, a fourth dose level of IMM60 of 24, 27, 30 33, or 3 mg per patient is administered for cycles.

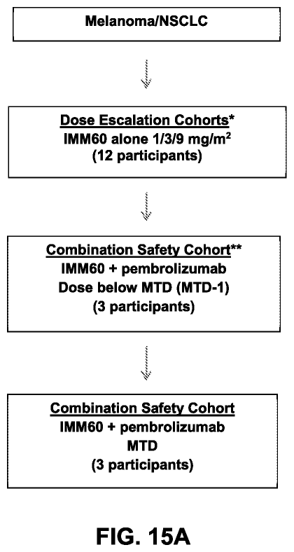

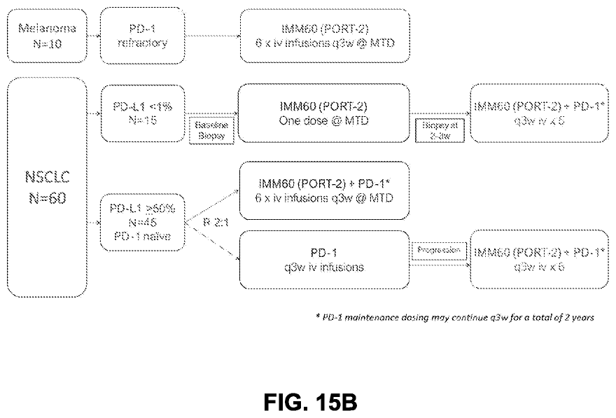

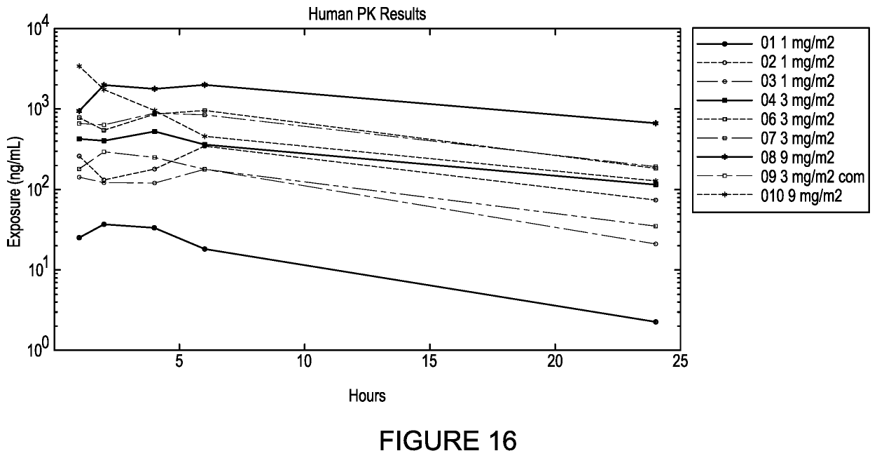

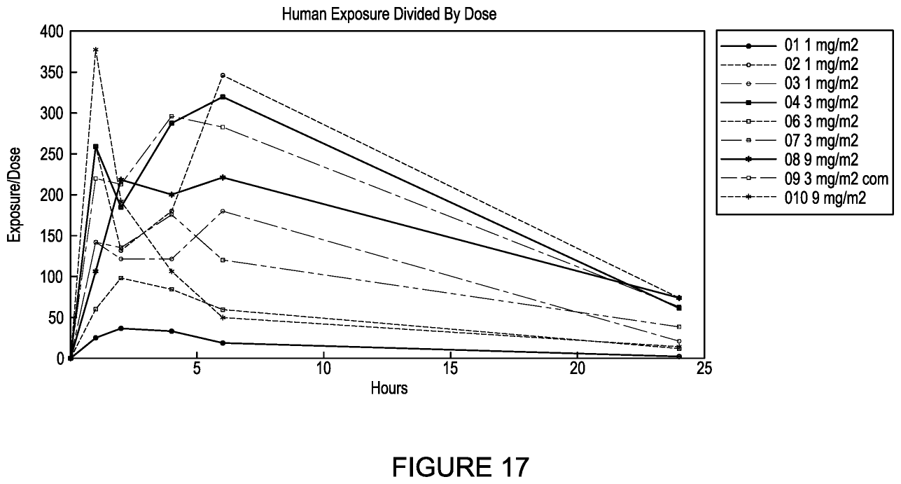

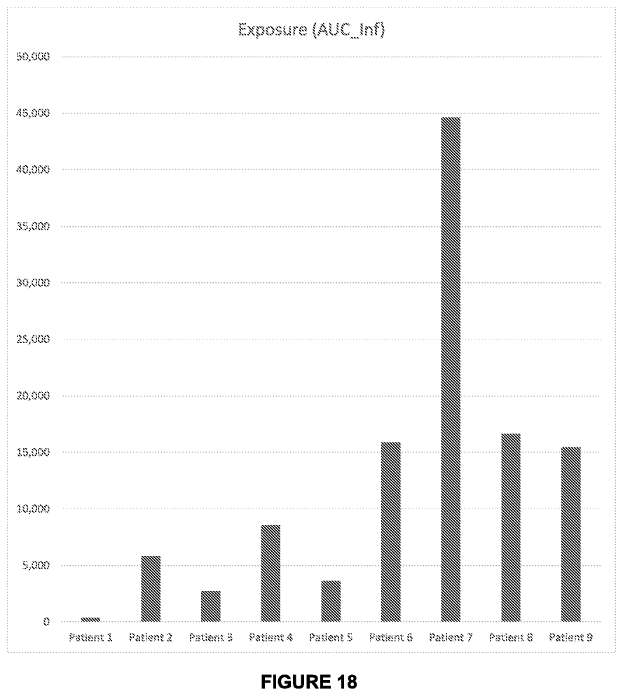

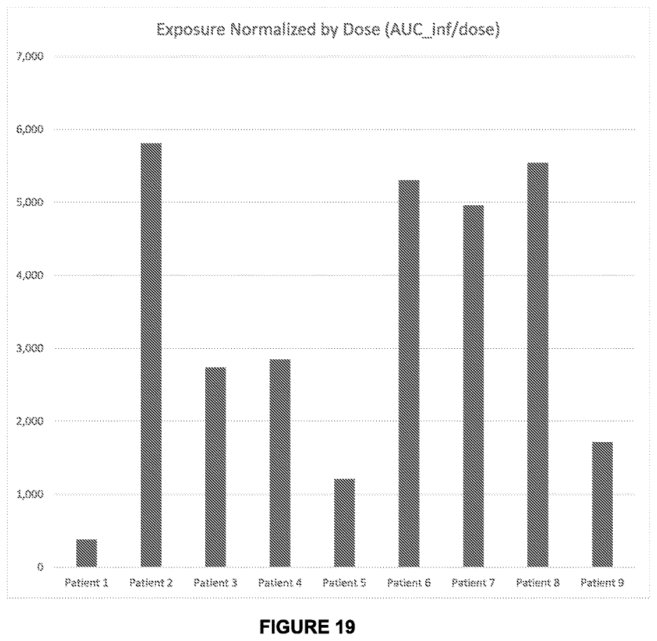

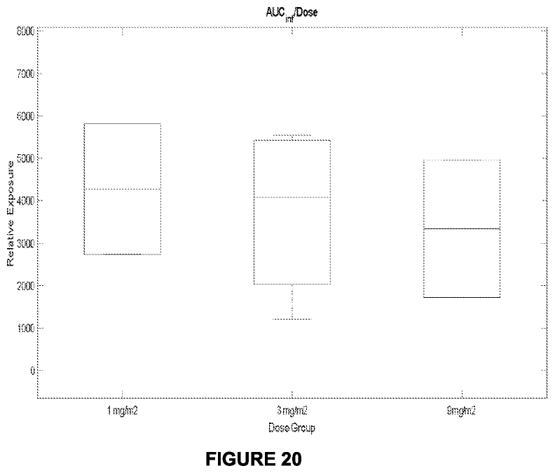

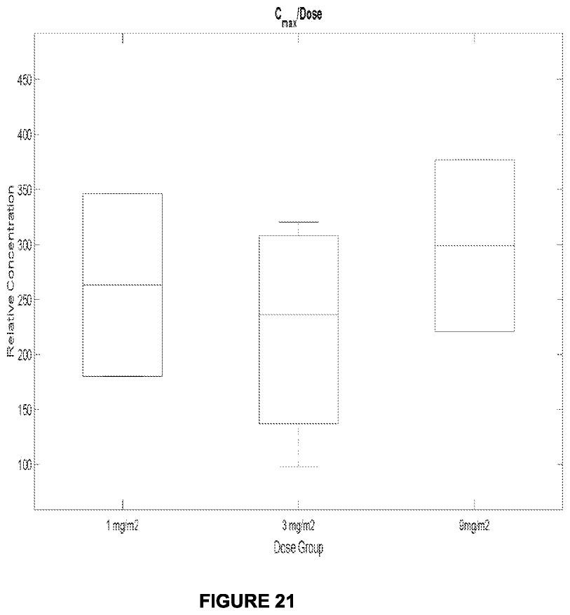

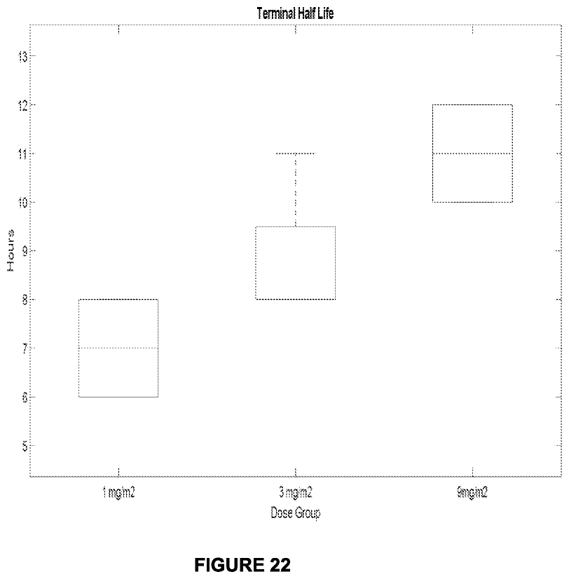

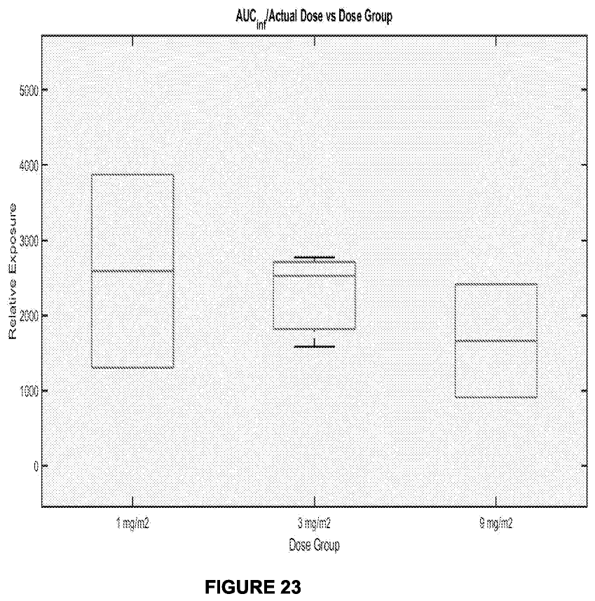

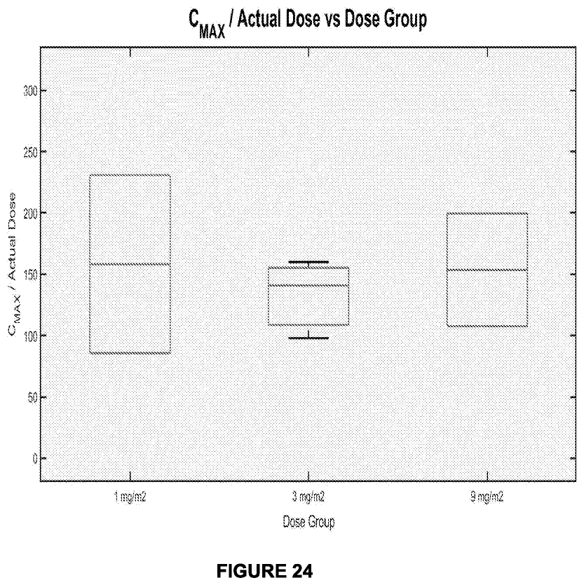

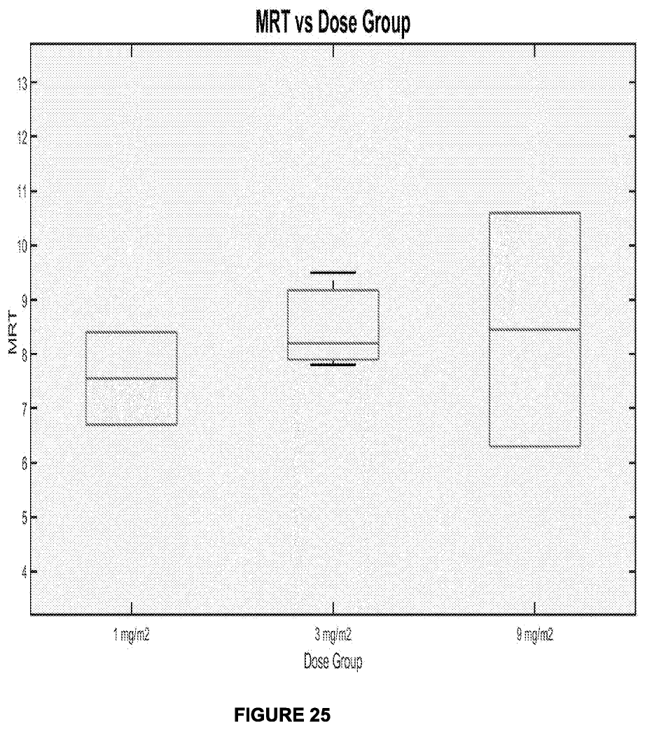

Pembrolizumab (MK-3475): 200 mg Q3W administered IV for up to 35 cycles or approximately 2 years
Condition/Disease/Target Population:
   Phase 1 (See FIG. 15A):

### NSCLC—Stage IV NSCLC

IMM60 dose escalation arm: Progression through systemic therapy consisting of at least platinum-based chemotherapy and immunotherapy (either sequentially or in combination)
IMM60+pembrolizumab dose safety arm: Previous chemotherapy and/or immunotherapy is permissible

Melanoma—unresectable stage III or IV cutaneous or unknown primary melanoma
IMM60 dose escalation arm: Patients progressing through at least one line of immunotherapy. For those patients with BRAF mutant tumors, progression through BRAF/mitogen-activated protein kinase inhibitor treatment and at least one line of immunotherapy
IMM60+pembrolizumab dose safety arm: Prior chemotherapy, targeted therapy, and/or immunotherapy are permitted; for those patients with BRAF mutant tumors, progression through BRAF/mitogen-activated protein kinase inhibitor treatment Phase 2 (See FIG. 15B):

### NSCLC—Stage IV NSCLC

Cohort 1: Metastatic NSCLC, PDL1+ve (PD-L1 Positive—Tumor Proportion Score [TPS]>50%; PD-L1 IHC 22C3 pharmDx); not previously exposed to a PD-1, PD-L1 inhibitor
Cohort 2: Metastatic NSCLC, PDL1−ve (PD-L1 Negative—TPS<1%; PD-L1 IHC 22C3 pharmDx); prior treatment with both chemotherapy and anti-PD-1/PD-L1 in the advanced setting. Previous chemotherapy and PD-1 treatment in the context of adjuvant treatment is permissible as long as the treatments were completed at least months prior to consent.

### Melanoma—Unresectable Stage III or IV Cutaneous or Unknown Primary Melanoma

Melanoma Cohort: Metastatic disease previously exposed to an anti-PD-1 or anti-PD-L1 agent

Study Duration: Participant duration on study is dependent on their treatment. Participants will be followed for overall survival (OS) for up to 3 years from last dose of treatment.

Treatment Duration: IMM60 is given in 3-week cycles with a maximum of cycles, pembrolizumab is given up to 35 cycles or approximately 2 years.

Follow-up Duration: Up to 1 year from first dose of treatment or until disease progression or death, whichever is sooner. Participants will be followed up for PFS and OS data via the medical care team for the duration of the follow-up period or until disease progression, further progression, or death, whichever is sooner.

End of study: Once the last participant has completed their follow-up period or until disease progression or death, whichever is sooner.

### Number of Participants:

85-100 evaluable participants will be enrolled in the study (phase 1: 9 to 18 participants in the initial dose-escalation part of the study and to 12 participants in the following safety part; phase 2: 70 participants [45 PD-L1>50% total NSCLC participants, 15 PD-L1<1% NSCLC participants, 10 PD-1 pretreated melanoma participants]). Should a dose limiting toxicity (DLT) occur in any of the phase 1 cohorts, then an additional 3 participants will be recruited to that dose level cohort.

### Study Arms and Duration:

### Phase 1:

The phase 1 study will be a standard 3+3 trial design in participants with advanced NSCLC or melanoma:

IMM60 dose escalation safety arm: 3 dose levels of IMM60 will be assessed (1/3/9 mg/m² administered IV). If one of the first 3 participants experiences a pre-defined DLT, that cohort will be expanded to a total of participants. If a further participant in this cohort experiences a DLT, then the MTD will be considered to be exceeded at this dose. If more than 1 DLT is observed in the participants in the expanded cohort then MTD will be defined as the dose level below that cohort. Should more than 1 DLT occur in the lowest IMM60 dose level cohort, the TMG will consider de-escalating this dose level. If no participant in the highest dose cohort (9 mg/m²) experiences a DLT, this cohort will still be expanded to participants.

IMM60+pembrolizumab combination safety arm: pembrolizumab will be administered in combination with IMM60. The dose of pembrolizumab will be that administered as per standard of care (200 mg Q3W administered IV); the IMM60 dose (administered IV) will be dependent on the IMM60 dose escalation arm. If no DLTs occur in the 1 mg/m² and 3 mg/m² IMM60 dose escalation cohorts, then the IMM60+pembrolizumab safety arm will open using the 3 mg/m²IMM60 dose level, and the combination safety arm will open in parallel to the 9 mg/m² IMM60 dose escalation cohort. If the combination safety arm opens in parallel to the 9 mg/m² IMM60 dose escalation cohort, then the combination safety arm will dose escalate to 9 mg/m² if <2 DLTs occur in the first combination safety cohort in phase 1. Should any DLT occur in the 1 mg/m² and 3 mg/m² IMM60 dose escalation cohorts, the IMM60 starting dose level for the combination safety arm will be one below IMM60 MTD (referred to as MTD-1) and will open after the completion of the 9 mg/m² dose escalation cohort and will dose escalate to a cohort with IMM60 at MTD.

Dose escalation will occur based on the toxicity information obtained from the evaluable participants in a cohort during the initial 21-day treatment cycle. The TMG will review the phase 1 data, the current standard of care, and feasibility to make a recommendation on phase 2 cohorts.

### Phase 2:

NSCLC Cohort 1: PD-L1 positive (TPS>50% PD-L1 IHC 22C3 pharmDx) NSCLC: Participants will be randomized 1:2 to either pembrolizumab as per its licensed indication (pembrolizumab alone, 200 mg Q3W administered IV) or the combination of IMM60+pembrolizumab 200 mg Q3W administered IV. Depending on review of IMM60 monotherapy activity, NSCLC participants in the pembrolizumab alone cohort who have radiographic progression with clinical deterioration or confirmed radiographic progression at two time points at least 4 weeks apart can then receive pembrolizumab+IMM60.

Additional planned non-randomized phase 2 cohorts include:

NSCLC Cohort 2: PD-L1 negative (TPS<1% PD-L1 IHC 22C3 pharmDx) NSCLC: Participants will be treated with one cycle of IMM60 with a tumor biopsy before and after, to determine any changes in PD-L1 expression. After this one cycle, the participants will receive the combination of IMM60+pembrolizumab 200 mg Q3W administered IV.

Melanoma Cohort: Participants with melanoma for whom treatment with a PD-1 inhibitor has failed will receive IMM60 monotherapy at the MTD given Q3W administered IV.

What is claimed is:

1. A liposome comprised of Compound A: 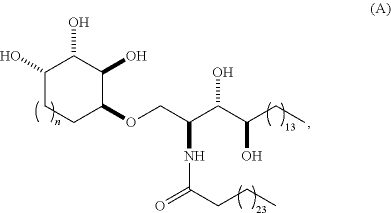
wherein n is 1 (IMM60), 2 (IMM70), or 3 (IMM80), or a salt, ester, solvate, or hydrate thereof, and two or more lipids or salts thereof;
wherein the two or more lipids comprise:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); or,
(f) 1,2-dimyristoyl-sn-glycero-3-phosphoglycerol (DMPG) and cholest-5-en-30-ol (CHOL).

2. The liposome of claim 1, wherein n is 1 (IMM60).

3. The liposome of claim 1 or 2, wherein Compound A is present in an amount of about 0.1 wt. % to about 20 wt. %, based on the total weight of the liposome.

4. The liposome of claim 1 or 2, wherein Compound A is present in an amount of about 0.5 wt. % to about 15 wt. %, or about 1 wt. % to about 12 wt. %, or about 5 wt % to about 12 wt. %, based on the total weight of the liposome.

5. The liposome of any one of claims 1 to 4, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.

6. The liposome of any one of claims 1 to 5, wherein the mass ratio of Compound A to the two or more lipids is about 1:5 to about 1:20, or about 1:7 to about 1:15, or about 1:7 to about 1:12, or about 1:9.

7. The liposome of any one of claims 1 to 6, wherein the two or more lipids comprise 1,2-distearoyl-sn-glycero-3-phosphocholine (DSPC) and 1,2-distearoyl-sn-glycero-3-phospho-rac-glycerol (DSPG).

8. The liposome of claim 7, wherein the DSPC is present in an amount of about 40 wt. % to 80 wt. %, or about 40 wt. % to about 60 wt. %, or about 45 wt. % to about 55 wt. %, based on the total weight of the liposome.

9. The liposome of claim 7 or 8, wherein the DSPG is present in an amount of about 20 wt. % to 60 wt. %, or about 30 wt. % to about 50 wt. %, or about 35 wt. % to about 45 wt. %, based on the total weight of the liposome.

10. The liposome of any one of claims 7-9, wherein the liposome further comprises cholest-5-en-30-ol (CHOL).

11. The liposome of claim 10, wherein the CHOL is present in an amount of about 1 wt % to about 20 wt %, or about 5 wt % to about 15 wt %, or about 8 wt % to about 12 wt %, based on the total weight of the liposome.

12. The liposome of claim 10 or 11, wherein the DSPC is present in an amount of about 40 wt % to about 50 wt %, the DSPG is present in an amount of about 30 wt % to about 40 wt %, and the CHOL is present in an amount of about 5 wt % to about 15 wt %.

13. The liposome of any one of claims 1 to 6, wherein the two or more lipids comprise 1-palmitoyl-2-oleoyl-sn-glycero-3-phosphocholine (POPC) and 3β-[N-(N′,N′-dimethylaminoethane)-carbamoyl]cholesterol hydrochloride (DC-Chol).

14. The liposome of claim 13, wherein the POPC is present in an amount of about 40 wt. % to 80 wt. %, or about 45 wt. % to about 65 wt. %, or about 50 wt. % to about 60 wt. %, based on the total weight of the liposome.

15. The liposome of claim 13 or 14, wherein the DC-Chol is present in an amount of about 15 wt. % to 55 wt. %, or about 20 wt. % to about 50 wt. %, or about 25 wt. % to about 35 wt. %, based on the total weight of the liposome.

16. The liposome of any one of claims 1 to 6, wherein the two or more lipids comprise 1-palmitoyl-2-oleoyl-sn-glycero-3-phosphocholine (POPC) and dimethyldioctadecylammonium bromide (DDAB).

17. The liposome of claim 16, wherein the POPC is present in an amount of about 40 wt. % to 80 wt. %, or about 45 wt. % to about 65 wt. %, or about 50 wt. % to about 60 wt. %, based on the total weight of the liposome.

18. The liposome of claim 1 or 17, wherein the DC-Chol is present in an amount of about 15 wt. % to 55 wt. %, or about 20 wt. % to about 50 wt. %, or about 30 wt. % to about 40 wt. %, based on the total weight of the liposome.

19. The liposome of any one of claims 1 to 6, wherein the two or more lipids comprise L-α-phosphatidylglycerol (EPG) and 1,2-dioleoyl-sn-glycero-3-ethylphosphocholine (EPC).

20. The liposome of claim 19, wherein the EPG is present in an amount of about 40 wt. % to 80 wt. %, or about 55 wt. % to about 75 wt. %, or about 60 wt. % to about 70 wt. %, based on the total weight of the liposome.

21. The liposome of claim 19 or 20, wherein the EPC is present in an amount of about 10 wt. % to 50 wt. %, or about 15 wt. % to about 40 wt. %, or about 15 wt. % to about 30 wt. %, based on the total weight of the liposome.

22. The liposome of any one of claims 1 to 6, wherein the two or more lipids comprise 1-palmitoyl-2-oleoyl-sn-glycero-3-phosphocholine (POPC) and 1,2-dioleoyl-3-trimethylammonium-propane (DOTAP).

23. The liposome of claim 22, wherein the POPC is present in an amount of about 40 wt. % to 80 wt. %, or about 40 wt. % to about 65 wt. %, or about 45 wt. % to about 55 wt. %, based on the total weight of the liposome.

24. The liposome of claim 22 or 23, wherein the DOTAP is present in an amount of about 20 wt. % to 50 wt. %, or about 25 wt. % to about 45 wt. %, or about 35 wt. % to about 45 wt. %, based on the total weight of the liposome.

25. The liposome of any one of claims 1 to 6, wherein the two or more lipids comprise 1,2-dimyristoyl-sn-glycero-3-phosphoglycerol (DMPG) and cholest-5-en-30-ol (CHOL).

26. The liposome of claim 25, wherein the DMPG is present in an amount of about 40 wt. % to 80 wt. %, or about 50 wt. % to about 75 wt. %, or about 60 wt. % to about 70 wt. %, based on the total weight of the liposome.

27. The liposome of claim 25 or 26, wherein the CHOL is present in an amount of about 10 wt. % to 50 wt. %, or about 15 wt. % to about 35 wt. %, or about 15 wt. % to about 30 wt. %, based on the total weight of the liposome.

28. The liposome of any one of claims 1 to 27, wherein the liposome further comprises a liposomal buffer.

29. The liposome of claim 28, wherein the liposomal buffer has a pH of about to 7, or about 6.3 to about 6.7.

30. The liposome of claim 29, wherein the liposomal buffer comprises NaCl and sodium phosphate.

31. The liposome of any one of claims 28-30, wherein the liposomal buffer further comprises sucrose.

32. The liposome of any one of claims 1 to 31, wherein the liposome further comprises at least one antigen.

33. The liposome of claim 32, wherein the at least one antigen comprises a member selected from the group consisting of a viral antigen, a bacterial antigen, a fungal antigen, a tumor antigen, and mixtures thereof.

34. The liposome of claim 32 or 33, wherein the at least one antigen comprises at least one tumor antigen.

35. The liposome of claim 34, wherein the tumor antigen is selected from the group consisting of:
(a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).

36. The liposome of any one of claims 31-35, wherein the antigen is present in the liposome in an amount of about 1 g of antigen to 1-20 g of Compound A.

37. The liposome of any one of claims 1 to 36, further comprising at least one therapeutic agent.

38. A composition comprising liposomes according to any one of claims 1-37 and a pharmaceutically acceptable diluent, excipient, adjuvant, or carrier.

39. The composition of claim 38, wherein the mean diameter of the liposomes is less than 200 nm.

40. The composition of claim 38, wherein the mean diameter of the liposomes is within a range of about 50 nm to 200 nm, or about 50 nm to about 170 nm, or about 75 nm to about 145 nm, or about 90 nm to about 130 nm.

41. The composition according to claim 38, wherein the mean diameter of the liposomes is 110 nm±60 nm or 110 nm±40 nm or 110 nm±20 nm.

42. The composition according to any one of claims 38 to 41, wherein the liposomes have a polydispersity index (PdI) of less than or equal to about 0.40, or less than or equal to about 0.35, or less than or equal to about 0.30, or less than or equal to about 0.25, or less than or equal to about 0.20, or less than or equal to about 0.15.

43. The composition of any one of claims 38 to 42, wherein the composition further comprises at least one antigen.

44. The composition of claim 43, wherein the at least one antigen comprises a member selected from the group consisting of a viral antigen, a bacterial antigen, a fungal antigen, a tumor antigen, and mixtures thereof.

45. The composition of claim 43 or 44, wherein the at least one antigen comprises at least one tumor antigen.

46. The composition of claim 45, wherein the tumor antigen is selected from the group consisting of:
(a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).

47. The composition of claim 45, wherein the tumor antigen comprises NY-ESO-1 or an antigenic fragment thereof.

48. The composition of any one of claims 38 to 47, further comprising at least one therapeutic agent.

49. The composition of claim 48, wherein the at least one therapeutic agent is selected from the group of an immune modulator, a Toll-like receptor agonist, a Nod ligand, an anti-viral agent, an antifungal agent, an antibiotic, an antiviral antibody, a cancer immune therapeutic, a chemotherapy agent, a kinase inhibitor, a cytotoxic agent, an anti-asthmatic agent, an antihistamine agent, an anti-inflammatory agent, a vaccine adjuvant, a second liposome, an artificial antigen presenting cell, a cytokine or chemokine blocking antibody, and combinations thereof.

50. A method of stimulating an immune response in a mammalian subject comprising administering to the subject the composition of any one of claims 38 to 49.

51. The method according to claim 50, further comprising a step, prior to the administering step, of heating the composition for a time and at a temperature sufficient to reduce average particle size or PdI of the liposomal composition.

52. The method according to claim 50, further comprising a step, prior to the administering step, of heating the composition for 5-15 minutes at a temperature of about 55 to about 65° C.

53. The composition according to any one of claims 38 to 49 for use in stimulating an immune response in a mammalian subject.

54. Use of the composition according to any one of claims 38 to 49 for stimulating an immune response in a mammalian subject.

55. The liposome according to any one of claims 1 to 37 or the composition according to any one of claims 38 to 49 for use in the manufacture of a medicament for stimulating an immune response in a mammalian subject.

56. The composition or use according to any one of claims 53 to 55, wherein the composition is subjected to a heating step prior to its use in stimulating the immune response.

57. The method or use of any one of claims 50-56, wherein the mammalian subject is human.

58. The method or use of any one of claims 50 to 57, wherein the mammalian subject has a cancer.

59. A method of treating a mammalian subject with cancer, comprising administering to the subject a therapeutically effective amount of the composition of any one of claims 38 to 49.

60. The method or use according to claim 58 or 59, wherein the composition is administered by subcutaneous, intravenous, or intratumoral injection.

61. The method according to claim 50 or 60, further comprising a step, prior to the administering step, of heating the composition for a time and at a temperature sufficient to reduce average particle size or PdI of the liposomal composition.

62. The liposome according to any one of claims 1 to 37 or the composition according to any one of claims 38 to 49 for use in the treatment of cancer in a mammalian subject.

63. The liposome according to any one of claims 1 to 37 or the composition according to any one of claims 38 to 49 for use in the manufacture of a medicament for treatment of cancer in a mammalian subject.

64. The composition or use according to any one of claims 61 to 62, wherein the composition is subjected to a heating step prior to its use in stimulating the immune response.

65. The method or use of any one of claims 58 to 64, wherein the cancer is selected from the group of basal cell carcinoma, breast cancer leukemia, Burkitt's lymphoma, colon cancer, esophageal cancer, bladder cancer, gastric cancer, head and neck cancer, hepatocellular cancer, Hodgkin's lymphoma, hairy cell leukemia, Wilms' tumor, thyroid cancer, thymoma and thymic carcinoma, testicular cancer, T-cell lymphoma, prostate cancer, non-small cell lung cancer, liver cancer, renal cell cancer, melanoma, and combinations thereof.

66. The method or use of any one of claims 58 to 64, wherein the cancer comprises melanoma.

67. The method or use of any one of claims 58 to 64, wherein the cancer comprises non-small cell lung cancer.

68. The method or use of any one of claims 58 to 67, further comprising administering to the subject or using at least one further therapeutic or therapy selected from the group consisting of a chemotherapeutic agent, a radiotherapeutic agent, and radiation therapy.

69. The method or use of any one of claims 58-68, further comprising administering to the subject or using at least one checkpoint inhibitor.

70. The method or use of claim 69, wherein the immune checkpoint inhibitor comprises a PD-1 inhibitor, a PD-L1 inhibitor, or a CTLA-4 inhibitor.

71. The method or use of claim 70, wherein the immune checkpoint inhibitor comprises at least one PD-1 inhibitor selected from Pembrolizumab, Nivolumab, Cemiplimab, and Spartalizumab.

72. The method or use of claim 70, wherein the immune checkpoint inhibitor comprises at least one PD-L1 inhibitor selected from Atezolizumab, Avelumab, and Durvalumab.

73. The method or use of claim 70, wherein the immune checkpoint inhibitor comprises at least one CTLA-4 inhibitor selected from Ipilimumab and Tremelimumab.

74. The method or use of any one of claims 68-73, wherein the composition and the further therapeutic or therapy are administered concurrently.

75. The method or use of any one of claims 68-73, wherein the composition and the further therapeutic or therapy are administered separately.

76. A method of preparing a liposome comprised of Compound A 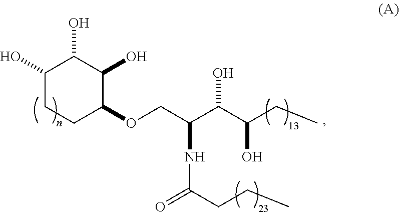
wherein n is 1 (IMM60), 2 (IMM70), or 3 (IMM80), or a salt, ester, solvate, or hydrate thereof, and two or more lipids or salts thereof: the method comprising:
(A) admixing Compound A and the two or more lipids to form multi-lamellar vesicles, wherein the two or more lipids comprise:
(a) 1,2-distearoyl-sn-glycero-3-phosphocholine (DSPC) and 1,2-distearoyl-sn-glycero-3-phospho-rac-glycerol (DSPG);
(b) 1-palmitoyl-2-oleoyl-sn-glycero-3-phosphocholine (POPC) and 3β-[N-(N′,N′-dimethylaminoethane)-carbamoyl]cholesterol hydrochloride (DC-Chol);
(c) 1-palmitoyl-2-oleoyl-sn-glycero-3-phosphocholine (POPC) and dimethyldioctadecylammonium bromide (DDAB);
(d) L-α-phosphatidylglycerol (EPG) and 1,2-dioleoyl-sn-glycero-3-ethylphosphocholine (EPC);
(e) 1-palmitoyl-2-oleoyl-sn-glycero-3-phosphocholine (POPC) and 1,2-dioleoyl-3-trimethylammonium-propane (DOTAP); or
(f) 1,2-dimyristoyl-sn-glycero-3-phosphoglycerol (DMPG) and cholest-5-en-30-ol (CHOL),
and,
(B) extruding the multi-lamellar vesicles through a membrane to form the liposomes.

77. The method according to claim 76, further comprising (c) purifying the liposomes.

78. The method according to claim 77, wherein the purifying comprises filtration.

79. The method according to claim 77, wherein the purifying comprises ultrafiltration, centrifugation, dialysis, diafiltration, or tangential flow filtration.

80. The method according to claim 78 that comprises filtering with a filter that has a pore size of 400 nm, or less than about 350 nm, or less than about 300 nm, or less than about 250 nm, or less than about 220 nm, or less than about 200 nm, or less than about 150 nm.

81. The method according to claim 78 that comprises filtering with a filter that has a pore size of about 50 nm to about 250 nm, or about 50 nm to about 150 nm, or about 60 nm to about 100 nm, or about 80 nm.

82. The method according to claim 78 that comprises filtering with a filter that has a molecular weight cutoff (MWCO) of 1000 kD or 800 kD or 600 kD or 500 kD or 400 kD or 300 kD or 250 kD.

83. The method of any one of claims 7-82, wherein n is 1 (IMM60).

84. The method of any one of claims 7-83, wherein Compound A is present in an amount of about 0.1 wt. % to about 20 wt. %, or about 0.5 wt. % to about 15 wt. %, or about 1 wt. % to about 12 wt. %, or about 5 wt % to about 12 wt. %, based on the total weight of the liposome.

85. The method of any one of claims 7-84, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.

86. The method of any one of claims 7-81, wherein the mass ratio of Compound A to the two or more lipids is in a range of about 1:5 to about 1:20, or about 1:7 to about 1:15, or about 1:7 to about 1:12, or about 1:9.

87. The method of any one of claims 7-86, wherein the two or more lipids comprise 1,2-distearoyl-sn-glycero-3-phosphocholine (DSPC) and 1,2-distearoyl-sn-glycero-3-phospho-rac-glycerol (DSPG).

88. The method of claim 87, wherein the DSPC is present in an amount of about 40 wt. % to 80 wt. %, or about 40 wt. % to about 60 wt. %, or about 45 wt. % to about 55 wt. %, based on the total weight of the liposome.

89. The method of claim 87 or 88, wherein the DSPG is present in an amount of about 20 wt. % to 60 wt. %, or about 30 wt. % to about 50 wt. %, or about 35 wt. % to about 45 wt. %, based on the total weight of the liposome.

90. The method of any one of claims 7-84, wherein the two or more lipids further comprise CHOL.

91. The method of any one of claims 7-90, wherein step (A) further includes admixing at least one antigen with the Compound A and the lipids.

92. The method of claim 91, wherein the at least one antigen comprises a viral antigen, a bacterial antigen, a fungal antigen, and/or a tumor antigen.

93. The method of claim 91 or 92, wherein the at least one antigen comprises at least one tumor antigen.

94. The liposome of claim 93, wherein the tumor antigen is selected from the group consisting of:
(a) P1A, MUC1, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, LAGE-1, SSX-1, SSX-2 (HOM-MEL-40), SSX-3, SSX-4, SSX-5, SCP-i, NY-ESO-1, PRAME, PSMA, tyrosinase, melan-A, XAGE,
(b) antigenic fragments of any of (a), and
(c) mixtures of any of (a) and/or (b).

95. The method of any one of claims 7-94 wherein step (A) further comprises admixing at least one therapeutic agent with the lipids and the Compound A.

96. A liposome prepared by the method of any one of the claims 76-95.

97. A liposome comprised of a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent,
wherein
the phospholipid bilayer is substantially devoid of cholesterol, plant sterols, and polyethylene glycolated lipids (PEG lipids).

98. The liposome of claim 1, wherein the phospholipid bilayer has a phase transition temperature above about 37° C.

99. The liposome of claim 97 or 98, wherein the lipid bilayer comprises DSPC and DSPG.

100. The liposome of claim 97 or 98, wherein the phospholipids in the phospholipid bilayer consist essentially of DSPC and DSPG.

101. The liposome of claim 1 or 2 that is free of phospholipids having a phase transition temperature below about 37° C.

102. The liposome of any one of claims 97-101 that is free of unsaturated lipids or unsaturated phospholipids.

103. The liposome of any one of claims 97-102 that is free of lipids or phospholipids having a chain length shorter than 16 carbon atoms.

104. The liposome of any one of claims 97-103, wherein the at least two phospholipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.

105. The liposome of any one of claims 97-104, further comprising a therapeutic agent.

106. The liposome of any one of claims 97-105, wherein the mass ratio of the at least one therapeutic agent to the at least two phospholipids is about 1:5 to about 1:20, or about 1:7 to about 1:15, or about 1:7 to about 1:12, or about 1:9.

107. The liposome of any one of claims 97-106 that has a mean diameter less than about 500 nm, or less than about 400 nm, or less than about 300 nm, or less than about 200 nm, or less than about 150 nm, or less than 125 nm, wherein the mean diameter is measured as a Z-average at 90°.

108. The liposome of any one of claim 97-107, wherein the at least one therapeutic agent comprises a hydrophobic or amphiphilic agent that intercalates or integrates into the phospholipid bilayer.

109. The liposome of any one of claims 97-108, wherein the at least one therapeutic agent includes a hydrophilic agent contained in the lumen of the liposome.

110. The liposome of any one of claims 97-109, wherein the at least one therapeutic agent is selected from small molecules, nucleic acids, RNA, DNA, aptamers, proteins, peptides, and a mixture thereof.

111. The liposome of any one of claims 97-110, wherein the at least one therapeutic agent comprises a ribonucleic acid (RNA).

112. The liposome of any one of claims 97-111, wherein the at least one therapeutic agent comprises an anti-cancer therapeutic.

113. The liposome of any one of claims 97-112, wherein the at least one therapeutic agent comprises an immunostimulant or encodes an immunostimulant.

114. The liposome of any one of claims 97-113, wherein the at least one therapeutic agent is present in an amount of about 0.1 wt. % to about 20 wt. % based on the total weight of the liposome.

115. The liposome of any one of claims 97-114, wherein the therapeutic agent is present in an amount of about 0.5 wt. % to about 15 wt. %, or about 1 wt. % to about 12 wt. %, or about 5 wt. % to about 12 wt. %, based on the total weight of the liposome.

116. The liposome of any one of claims 97-115, comprising one or more therapeutic agent.

117. The liposome of any one of claims 97-116, wherein the lumen comprises a therapeutic agent.

118. A composition comprising liposomes according to any one of claims 97-117 and a pharmaceutically acceptable carrier.

119. The composition according to claim 118, wherein the liposomes have a mean diameter of less than or equal to about 500 nm, or less than about 400 nm, or less than about 300 nm, or less than about 200 nm, or less than about 150 nm, or less than 125 nm, wherein the mean diameter is measured as a Z-average at 90°.

120. The composition according to claim 118 or 119, wherein the liposomes have a mean diameter of about 100 nm±60 nm or about 100 nm±40 nm or about 100 nm±20 nm.

121. The composition of any one of claims 118-120, wherein the liposomes have a terminal half-life of at least about 6 hours after the composition is administered to humans.

122. The composition of any one of claims 118-121, wherein the liposomes have a terminal half-life of about 6-12 hours after the composition is administered to humans intravenously.

123. The composition of any one of claims 118-122, wherein the liposomes have a terminal half-life of about 8 hours after the composition is administered to humans intravenously.

124. The composition of any one of claims 118-123, wherein the liposome has a steady state volume of distribution in humans between about 2 liters to about 12 liters, or about 2 liters to about 10 liters, or about 2 liters to about 8 liters, after the composition is administered to humans intravenously.

125. The composition of any one of claims 118-124, wherein the liposome has a terminal volume of distribution in a mammalian subject of about 3 liters to about 10 liters after the composition is administered intravenously.

126. The composition of any one of claims 118-125, wherein the composition provides a steady plasma concentration of therapeutic agent between about 2 hours to about 10 hours, or between about 3 hours to about 8 hours, or between about 4 hours to about 6 hours, after administration to a mammalian subject.

127. The composition of any one of claims 118-126, wherein the composition provides a steady plasma concentration of therapeutic agent between about 4 hours and about 6 hours after administration to a mammalian subject.

128. The composition of any one of claims 118-127, wherein the therapeutic agent is not substantially cleared from plasma between about 4 hours and about 6 hours after administration to a mammalian subject.

129. The composition of any one of claims 118-128, wherein the mammalian subject is human.

130. A method of stimulating an immune response in a mammalian subject comprising administering to the subject the composition of any one of claims 118-129.

131. Use of the composition of any one of claims 118-129 for stimulating an immune response in a mammalian subject.

132. The liposome of any one of claims 96-117 or the composition of any one of claims 118-129 for use in the manufacture of a medicament for stimulating an immune response in a mammalian subject.

133. The method or use of any one of claims 130-132, wherein the mammalian subject is human.

134. The method or use of any one of claims 130-133, wherein the mammalian subject has a cancer.

135. A method of treating a mammalian subject with cancer, comprising administering to the subject a therapeutically effective amount of the composition of any one of claims 118-129.

136. The method or use of claim 134 or method of 135, wherein the composition is administered by subcutaneous, intravenous, or intratumoral injection.

137. The liposome of any one of claims 97-117 or the composition of any one of claims 118-129 for use in the treatment of cancer in a mammalian subject.

138. The liposome of any one of claims 97-117 or the composition of any one of claims 118-129 for use in the manufacture of a medicament for treatment of cancer in a mammalian subject.

139. The method or use of any one of claims 134-138, wherein the cancer is selected from the group of basal cell carcinoma, breast cancer leukemia, Burkitt's lymphoma, colon cancer, esophageal cancer, bladder cancer, gastric cancer, head and neck cancer, hepatocellular cancer, Hodgkin's lymphoma, hairy cell leukemia, Wilms' tumor, thyroid cancer, thymoma and thymic carcinoma, testicular cancer, T-cell lymphoma, prostate cancer, pancreatic cancer, non-small cell lung cancer, liver cancer, renal cell cancer, melanoma, and combinations thereof.

140. The method or use of any one of claims 134-139, wherein the cancer comprises or is melanoma.

141. The method or use of any one of claims 134-140, wherein the cancer comprises or is non-small cell lung cancer.

142. The method or use of any one of claims 134-141, wherein the cancer comprises or is liver cancer.

143. The method or use of any one of claims 134-142, further comprising administering to the subject or using at least one further therapeutic or therapy selected from the group consisting of a chemotherapeutic agent, a radiotherapeutic agent, and radiation therapy.

144. The method or use any one of claims 134-143, further comprising administering to the subject or using at least one immune checkpoint inhibitor.

145. The method or use of claim 144, wherein the immune checkpoint inhibitor comprises a PD-1 inhibitor, a PD-L1 inhibitor, or a CTLA-4 inhibitor.

146. The method or use of claim 145, wherein the immune checkpoint inhibitor comprises at least one PD-1 inhibitor selected from Pembrolizumab, Nivolumab, Cemiplimab, and Spartalizumab.

147. The method or use of claim 145, wherein the immune checkpoint inhibitor comprises at least one PD-L1 inhibitor selected from Atezolizumab, Avelumab, and Durvalumab.

148. The method or use of claim 145, wherein the immune checkpoint inhibitor comprises at least one CTLA-4 inhibitor selected from Ipilimumab and Tremelimumab.

149. The method or use of any one of claims 143-148, wherein the composition and the further therapeutic or therapy are administered concurrently.

150. The method or use of any one of claims 143-148, wherein the composition and the further therapeutic or therapy are administered separately.

151. A method of preparing a liposome comprising a phospholipid bilayer and a lumen, the phospholipid bilayer comprising (a) at least two phospholipids selected from 1,2-distearoyl-sn-glycero-3-phosphocholine (DSPC), 1,2-distearoyl-sn-glycero-3-phospho-rac-glycerol (DSPG), 1,2-dipalmitoyl-sn-glycero-3-phosphocholine (DPPC), and 1,2-dipalmitoyl-sn-glycero-[phosphor-rac-(1-glycerol)](DPPG); and (b) at least one therapeutic agent,
wherein the phospholipid bilayer is substantially devoid of cholesterol, plant sterols, and polyethylene glycolated lipids (PEG lipids),
wherein the method comprises 1) admixing the therapeutic agent and the at least two phospholipids to form multi-lamellar vesicles and 2) extruding the multi-lammellar vesicles through a membrane to form the liposomes.

152. The method of claim 151, further comprising purifying the liposomes.

153. The method of claim 151 or 152, wherein purifying the liposomes comprises filtration.

154. The method of claim 153, wherein purifying comprises ultrafiltration, centrifugation, dialysis, diafiltration, or tangential flow filtration.

155. The method according to any one of claims 151-154 that comprises filtering with a filter that has a pore size of about 450 nm or less, or about 400 nm or less, or about 350 nm or less, or about 300 nm or less, or about 250 nm or less, or about 220 nm or less, or about 200 nm or less, or about 150 nm or less.

156. The method according to any one of claims 151-155, wherein the membrane has a pore size of about 50 nm to about 250 nm, or about 50 nm to about 150 nm, or about 60 nm to about 100 nm, or about 80 nm.

157. The method according to any one of claims 151-156 that comprises filtering with a filter that has a molecular weight cutoff (MWCO) of about 500 kD, or about 450 kD, or about 400 kD, or about 350 kD, or about 300 kD, or about 250 kD.

158. The method of any one of claims 151-157, wherein the therapeutic agent is present in an amount of about 0.1 wt. % to about 20 wt. %, or about 0.5 wt. % to about 15 wt. %, or about 1 wt. % to about 12 wt. %, or about 5 wt % to about 12 wt. %, based on the total weight of the liposome.

159. The method of any one of claims 151-158, wherein the two or more lipids comprise about 75 wt. % to about 99.9 wt. %, or about 80 wt. % to about 98 wt. %, or about 80 wt. % to about 95 wt. %, or about 85 wt. %, to about 95 wt. % based on the total weight of the liposome.

160. The method of any one of claims 151-159, wherein the mass ratio of the therapeutic agent is in a range of about 1:5 to about 1:20, or about 1:7 to about 1:15, or about 1:7 to about 1:12, or about 1:9.

161. The method of any one of claims 151-160, wherein the two or more lipids comprise 1,2-distearoyl-sn-glycero-3-phosphocholine (DSPC) and 1,2-distearoyl-sn-glycero-3-phospho-rac-glycerol (DSPG).

162. The method of claim 161, wherein the DSPC is present in an amount of about 40 wt. % to 80 wt. %, or about 40 wt. % to about 60 wt. %, or about 45 wt. % to about 55 wt. %, based on the total weight of the liposome.

163. The method of claim 161 or 162, wherein the DSPG is present in an amount of about 20 wt. % to 60 wt. %, or about 30 wt. % to about 50 wt. %, or about 35 wt. % to about 45 wt. %, based on the total weight of the liposome.

164. The method of any one of claims 151-163, wherein step 1) further includes admixing at least one antigen with the therapeutic agent and the lipids.

165. The method of claim 164, wherein the at least one antigen comprises a viral antigen, a bacterial antigen, a fungal antigen, and/or a tumor antigen.

166. The method of claim 164 or 165, wherein the at least one antigen comprises at least one tumor antigen.

167. A method of treating a mammalian subject with an immune checkpoint inhibitor, the method comprising:
(a) identifying a mammalian subject as having a cancer that is insensitive to treatment with an immune checkpoint inhibitor that targets the Programmed Cell Death Protein 1 (PD-1) pathway;
(b) administering to the subject a composition comprising IMM60, in an amount effective to increase sensitivity of the cancer to the immune checkpoint inhibitor; and
(c) administering to the subject a composition comprising the immune checkpoint inhibitor following the administration of the composition comprising IMM60.

168. A method of sensitizing a cancer in a mammalian subject to treatment with an immune checkpoint inhibitor, the method comprising:
identifying a subject as having a cancer that is insensitive to treatment with an immune checkpoint inhibitor that targets the Programmed Cell Death Protein 1 (PD-1) pathway; and
administering to the subject a composition comprising IMM60 in an amount and for a duration effective to sensitize the cancer to treatment with the immune checkpoint inhibitor.

169. A composition comprising IMM60 for use in increasing the expression of PD-L1 in cancer cells of a subject identified as having a PD-L1-negative cancer.

170. The composition of claim 169 for use in an ordered combination treatment regimen for cancer, said treatment regimen further comprising subsequent use of a composition comprising an immune checkpoint inhibitor that targets the Programmed Cell Death Protein 1 (PD-1) pathway.

171. IMM60 and a PD-L1 inhibitor for use as a combination therapy for treatment of cancer in a subject having a PD-L1-negative cancer.
